(19) United States
(12) Patent Application Publication
(10) Pub. No.: US 2022/0175722 A1
(43) Pub. Date: Jun. 9, 2022

(54) DEGRADERS OF FIBROBLAST GROWTH FACTOR RECEPTOR 2 (FGFR2)

(71) Applicant: DANA-FARBER CANCER INSTITUTE, INC., Boston, MA (US)

(72) Inventors: Nathanael Gray, Stanford, CA (US); Tinghu Zhang, Brookline, MA (US); Guangyan Du, Roslindale, MA (US); Nathaniel Henning, Brookline, MA (US); Jie Jiang, Brookline, MA (US)

(73) Assignee: DANA-FARBER CANCER INSTITUTE, INC., Boston, MA (US)

(21) Appl. No.: 17/601,605

(22) PCT Filed: Apr. 9, 2020
(86) PCT No.: PCT/US2020/027412

§ 371 Date: Oct. 5, 2021

Related U.S. Application Data
(60) Provisional application No. 62/831,952, filed on Apr. 10, 2019.
(60) Provisional application No. 62/884,422, filed on Aug. 8, 2019.

(51) Int. Cl. A61K 31/403; A61K 31/426; A61K 31/473; A61K 31/519; A61K 47/54; A61K 47/55

(52) U.S. Cl. CPC A61K 31/403; A61K 31/426; A61K 47/55; A61K 31/519; A61K 47/555; A61K 31/473

(57) ABSTRACT

The present invention relates to bispecific compounds, compositions, and methods for treating diseases or conditions characterized or mediated by aberrant fibroblast growth factor receptor 2 (FGFR2) activity.

### RELATED APPLICATIONS

This application claims the benefit of priority under 35 U.S.C. § 119(e) to U.S. Provisional Application No. 62/831,952, filed Apr. 10, 2019 and U.S. Provisional Application No. 62/884,422, filed Aug. 8, 2019, each of which are incorporated herein by reference in their entireties.

### GOVERNMENT LICENSE RIGHTS

This invention was made with government support under grant number P50 CA127003 awarded by the National Institutes of Health. The government has certain rights in the invention.

### BACKGROUND OF THE INVENTION

Biliary tract cancers (BTC) account for approximately 3% of all gastro-intestinal (GI) malignancies and carry poor prognosis. The 5-year survival rate for all patients is less than 15%, and falls to 10% for stage III and nearly 0% for stage IV, despite treatment with the best available chemotherapy. The incidence of BTC has been increasing for several decades, primarily due to the rise in intrahepatic cholangiocarcinoma (ICC). Genetic alterations that activate fibroblast growth factor (FGF) signaling are among the most common genomic changes in ICC, present in greater than 20% of tumors, and most frequently involve fusion of fibroblast growth factor receptor 2 (FGFR2) exons 1-18 with a variety of partners encoding dimerization domains, resulting in constitutive FGFR2 kinase activity.

FGFRs play important roles in many biological processes such as tissue repair, hematopoiesis, bone growth, angiogenesis, and regulating metabolic processes. The emergence of FGFR2 as a therapeutic target provides new hope for patients with ICC. The most advanced FGFR-selective compound in clinical development for ICC, BGJ398, demonstrated efficacy in a phase II trial of patients with advanced refractory ICC that harbored an FGFR alteration (fusions, amplifications, or point mutations). The overall response rate (ORR) was 14.8% (18.8% in FGFR2 fusions only) and the disease control rate (DCR; i.e. partial responses+stable disease) was 75.4% (83.3% in FGFR2 fusions only). Median progression free survival was 5,8 months. While 5,8-month survival remains unacceptably low, these results are clearly superior to historical control data in patients with refractory advanced ICC.

However, a number of challenges exist for translating this encouraging clinical signal into long-term clinical benefit. The first challenge is acquired resistance to adenosine triphosphate (ATP) competitive FGFR inhibitors. The second challenge is on-target toxicity of inhibiting different members of the FGFR family. Given these challenges, fundamentally new approaches are needed to achieve transformational advances in the targeting of FGFRs.

### SUMMARY OF THE INVENTION

A bispecific compound having a structure represented by formula (I):

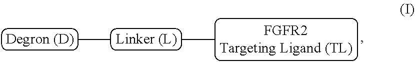

wherein the targeting ligand represents a moiety that binds fibroblast growth factor receptor 2 (FGFR2), the degron represents a moiety that binds an E3 ubiquitin ligase, and the linker represents a moiety that covalently connects the degron and the targeting ligand, or a pharmaceutically acceptable salt or stereoisomer thereof.

Another aspect of the present invention is directed to a pharmaceutical composition containing a therapeutically effective amount of the bispecific compound or a pharmaceutically acceptable salt or stereoisomer thereof, and a pharmaceutically acceptable carrier.

In another aspect of the present invention, methods of making the bispecific compounds are provided.

A further aspect of the present invention is directed to a method of treating a disease or disorder characterized or mediated by aberrant FGFR2 activity, that includes administering a therapeutically effective amount of the bispecific compound or a pharmaceutically acceptable salt or stereoisomer thereof, to a subject in need thereof.

Without intending to be bound by any particular theory of operation, the bispecific compounds of formula (I) (also referred to herein as degraders) are believed to promote the degradation of FGFR2 while sparing other FGFR isoforms. By conjugating low nanomole potency of pan-FGFR2 ligands with an E3 ligase binder, these bispecific compounds may fast recruit E3 ligase, and therefore promote the degradation of FGFR2. The degraders may achieve high target selectivity beyond that expected from the constitutive binding ligands, thus greatly reducing off-target effects.

Accordingly, the bispecific compounds of the present invention may serve as a set of new chemical tools for FGFR2 knockdown, exemplify a broadly applicable approach to arrive at degraders that are selective over non-selective binding ligands, and may provide effective treatments for FGFR2-mediated diseases and disorders such as cancer.

### BRIEF DESCRIPTION OF THE DRAWINGS

FIG. 1A-FIG. 1C are immunoblots that show the selective knockdown of FGFR2. FIG. 1A is an immunoblot that shows the knockdown of FGFR2 in Kato III cells after 16 hours at various concentrations for bispecific compound 6. FIG. 1B is an immunoblot that shows the knockdown of FGFR2 in Kato III cells after 4 hours for bispecific compound 6, BGJ398, control-1, bortezomib, and MLN4924. FIG. 1C is an immunoblot that shows the knockdown of FGFR2 in Kato III cells over a period of 16 hours at concentrations of 0.1 μM and 0.5 μM for bispecific compound 6.

FIG. 2A-FIG. 2C are immunoblots that show FGFR1/3/4 degradation. FIG. 2A is an immunoblot that shows the knockdown of FGFR1 in cholangiocarcinoma cells (CCLP1) after 16 hours at various concentrations for bispecific compound 6. FIG. 2B is an immunoblot that shows the knockdown of FGFR3/4 in hepatocellular carcinoma cells (JHH7) after 16 hours at various concentrations for bispecific compound 6. FIG. 2C is an immunoblot that shows the knockdown of FGFR1 in CCLP1 cells over a period of 16 hours at concentrations of 0.1 μM and 0.5 μM for bispecific compound 6.

FIG. 3A and FIG. 3B are graphs that show cell viability for FGFR2 degraders. FIG. 3A is a graph showing cell viability for VHL-based FGFR2 degrader. FIG. 3B is a graph showing cell viability for CRBN-based FGFR2 degraders.

FIG. 4A is an immunoblot that shows the degradation of FGFR2 in Kato III cells after 4 hours at various concentrations for bispecific compounds 6 and 7 (negative control).

FIG. 4B is a graph showing cell viability for bispecific compounds 6 and 7 (negative control) and BGJ398 with corresponding IC₅₀ values.

FIG. 5A and FIG. 5B are immunoblots that show FGFR2 degradation. FIG. 5A is an immunoblot that shows the knockdown of FGFR2 in Kato III cells after 6 hours at 1 μM concentration for bispecific compounds 6 and 14-22. FIG. 5B is an immunoblot that shows the degradation of FGFR2 in Kato III cells after 4 hours at different concentrations for bispecific compound 20, FIIN2, control-1, bortezomib and MLN4924.

FIG. 6A and FIG. 6B are immunoblots that show the knockdown of FGFR1 and FGFR4, respectively. FIG. 6A is an immunoblot that shows the knockdown of FGFR1 in CCLP1 cells after 6 hours at 1 μM concentration for bispecific compounds 6 and 14-22. FIG. 6B is an immunoblot that shows the knockdown of FGFR4 in JHH7 cells after 6 hours at 1 μM concentration for bispecific compounds 6 and 14-22.

### DETAILED DESCRIPTION

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present invention.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

With respect to bispecific compounds of the present invention, and to the extent the following terms are used herein to further describe them, the following definitions apply.

As used herein, the term “alkyl” refers to a saturated linear or branched-chain monovalent hydrocarbon radical. In one embodiment, the alkyl radical is a C₁-C₁₈ group. In other embodiments, the alkyl radical is a C₀-C₆, C₀-C₅, C₀-C₃, C₁-C₂, C₁-C₈, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group.

The terms “alkoxyl” or “alkoxy” as used herein refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl.

As used herein, the term “halogen” (or “halo” or “halide”) refers to fluorine, chlorine, bromine, or iodine.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkynyl” refers to a linear or branched monovalent hydrocarbon radical with at least one carbon-carbon triple bond. In one example, the alkynyl radical is a C₂-C₁₈ group. In other examples, the alkynyl radical is C₂-C₁₂, C₂-C₁₀, C₂-C₈, C₂-C₆ or C₂-C₃. Examples include ethynyl prop-1-ynyl, prop-2-ynyl, but-1-ynyl, but-2-ynyl and but-3-ynyl.

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Thus, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Thus, the term carbocyclic also embraces carbocyclylalkyl groups which as used herein refer to a group of the formula —R^{c}-carbocyclyl where R^{c} is an alkylene chain. The term carbocyclic also embraces carbocyclylalkoxy groups which as used herein refer to a group bonded through an oxygen atom of the formula —O—R^{c}-carbocyclyl where R^{c} is an alkylene chain.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur or oxygen. In some embodiments, heterocyclyl includes 4- to 6-membered monocycles having one or more heteroatoms selected from nitrogen, sulfur or oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Examples of 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine

N-oxides and the pyridyl, pyrimid-2-yl, pyrimid-4-yl, pyridazinyl and the 1,3,4-triazin-2-yl groups, are yet other examples of heterocyclyl groups. In some embodiments, a heterocyclic group includes a heterocyclic ring fused to one or more (e.g., 1, 2 or 3) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}— heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring.

Thus, the term aryl embraces aralkyl groups (e.g., benzyl) which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl. 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

Thus, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula —R^{c}-heteroaryl, wherein R^{c} is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

Any of the groups described herein may be substituted or unsubstituted. As used herein, and to the extent they are not otherwised defined for any particular group, the term “substituted” broadly refers to all permissible substituents with the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc. Representative substituents include halogens, hydroxyl groups, and any other organic groupings containing any number of carbon atoms, e.g., 1-14 carbon atoms, and which may include one or more (e.g., 1 2 3, or 4) heteroatoms such as oxygen, sulfur, and nitrogen grouped in a linear, branched, or cyclic structural format.

Representative examples of substituents may thus include alkyl, substituted alkyl (e.g., C₁-C₆, C₁-C₅, C₁-C₄, C₁-C₃, C₁-C₂, C₁), alkoxy (e.g., C₁-C₆, C₁-C₅, C₁-C₄, C₁-C₃, C₁-C₂, C₁), substituted alkoxy (e.g., C₁-C₆, C₁-C₅, C₁-C₄, C₁-C₃, C₁-C₂, C₁), haloalkyl (e.g., CF₃), alkenyl (e.g., C₂-C₆, C₂-C₅, C₂-C₄, C₂-C₃, C₂), substituted alkenyl (e.g., C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., C₂-C₆, C₂-C₅, C₂-C₄, C₂-C₃, C₂), cyclic (e.g., C₃-C₁₂, C₅-C₆), substituted cyclic (e.g., C₃-C₁₂, C₅-C₆), carbocyclic (e.g., C₃-C₁₂, C₅-C₆), substituted carbocyclic (e.g., C₃-C₁₂, C₅-C₆), heterocyclic (e.g., C₃-C₁₂, C₅-C₆), substituted heterocyclic (e.g., C₃-C₁₂, C₅-C₆), aryl (e.g., benzyl and phenyl), substituted aryl (e.g., substituted benzyl or phenyl), heteroaryl (e.g., pyridyl or pyrimidyl), substituted heteroaryl (e.g., substituted pyridyl or pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., C₆-C₁₂, C₆), alkylthio (e.g., C₁-C₆), substituted alkylthio (e.g., C₁-C₆), arylthio (e.g., C₆-C₁₂, C₆), substituted arylthio (e.g., C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

The term “binding” as it relates to interaction between the targeting ligand and the targeted protein or proteins, which in this invention is FGFR2, typically refers to an inter-molecular interaction that may be preferential or substantially specific (also referred to herein as “selective”) in that binding of the targeting ligand with other proteinaceous entities present in the cell is functionally insignificant. The present bispecific compounds may preferentially bind and recruit FGFR2 for targeted degradation.

The term “binding” as it relates to interaction between the degron and the E3 ubiquitin ligase, typically refers to an inter-molecular interaction that may or may not exhibit an affinity level that equals or exceeds that affinity between the targeting ligand and the target protein, but nonetheless wherein the affinity is sufficient to achieve recruitment of the ligase to the targeted degradation and the selective degradation of the targeted protein.

Broadly, the bispecific compounds have a structure represented by formula:

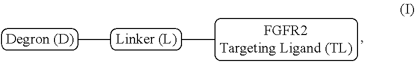

wherein the targeting ligand represents a moiety that binds fibroblast growth factor receptor 2 (FGFR2), the degron represents a moiety that binds an E3 ubiquitin ligase, and the linker represents a moiety that covalently connects the degron and the targeting ligand, or a pharmaceutically acceptable salt or stereoisomer thereof.

### FGFR2 Targeting Ligands

In some embodiments, the targeting ligand has a structure represented by formula (TL-1):

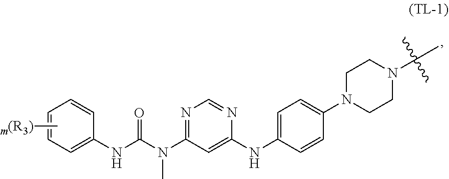

wherein
R₃ is independently halo, optionally substituted alkyl, optionally substituted alkoxy, optionally substituted amino, optionally substituted amido, carboxyl, acrylamide, optionally substituted carbocyclyl, or optionally substituted heterocyclyl; and m is an integer of 0-4.

In some embodiments, R₃ is independently methyl, chloro, or methoxy.

In some embodiments, m is 0.

In some embodiments, m is 2.

In some embodiments, m is 4.

Thus, in some embodiments, the bispecific compounds of the present invention have a structure represented by formula (I-1):

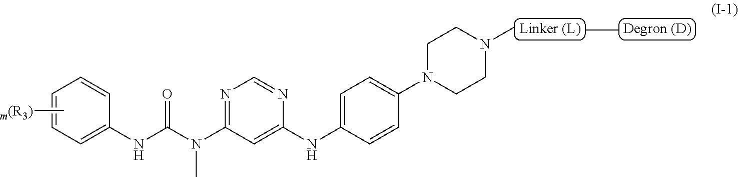

pharmaceutically acceptable salt or stereoisomer thereof.

In some embodiments, the targeting ligand has a structure represented by formula TL-1a, TL-1b, TL-1c, TL-1d, or TL-1e:

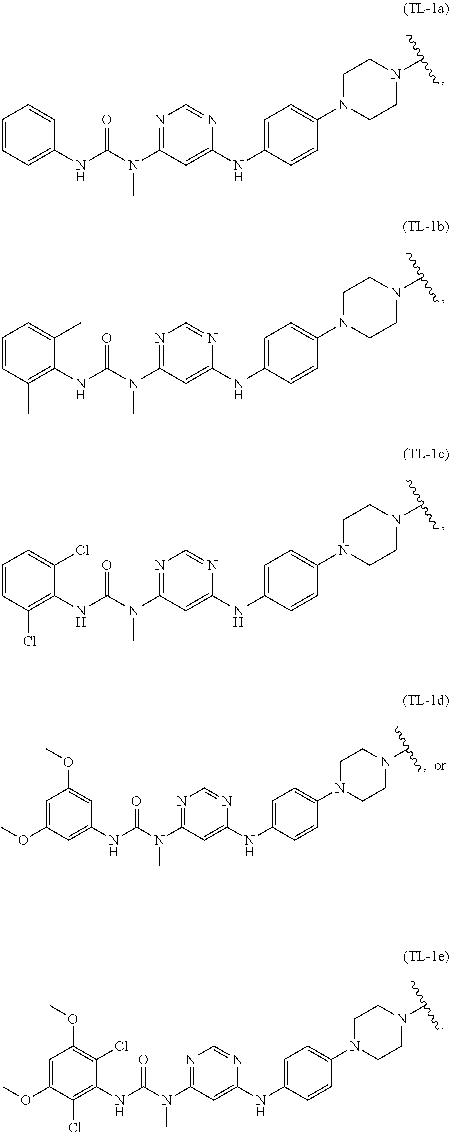 [SMILES: *N1CCN(c2ccc(Nc3cc(N(C)C(=O)Nc4ccccc4)ncn3)cc2)CC1 ; *N1CCN(c2ccc(Nc3cc(N(C)C(=O)Nc4c(C)cccc4C)ncn3)cc2)CC1 ; *N1CCN(c2ccc(Nc3cc(N(C)C(=O)Nc4c(Cl)cccc4Cl)ncn3)cc2)CC1 ; *N1CCN(c2ccc(Nc3cc(N(C)C(=O)Nc4cc(OC)cc(OC)c4)ncn3)cc2)CC1 ; *N1CCN(c2ccc(Nc3cc(N(C)C(=O)Nc4c(Cl)c(OC)cc(OC)c4Cl)ncn3)cc2)CC1]

Thus, in some embodiments, the bispecific compounds of the present invention have a structure represented by formula I-1a, I-1b, I-1c, I-1d, or I-1e:

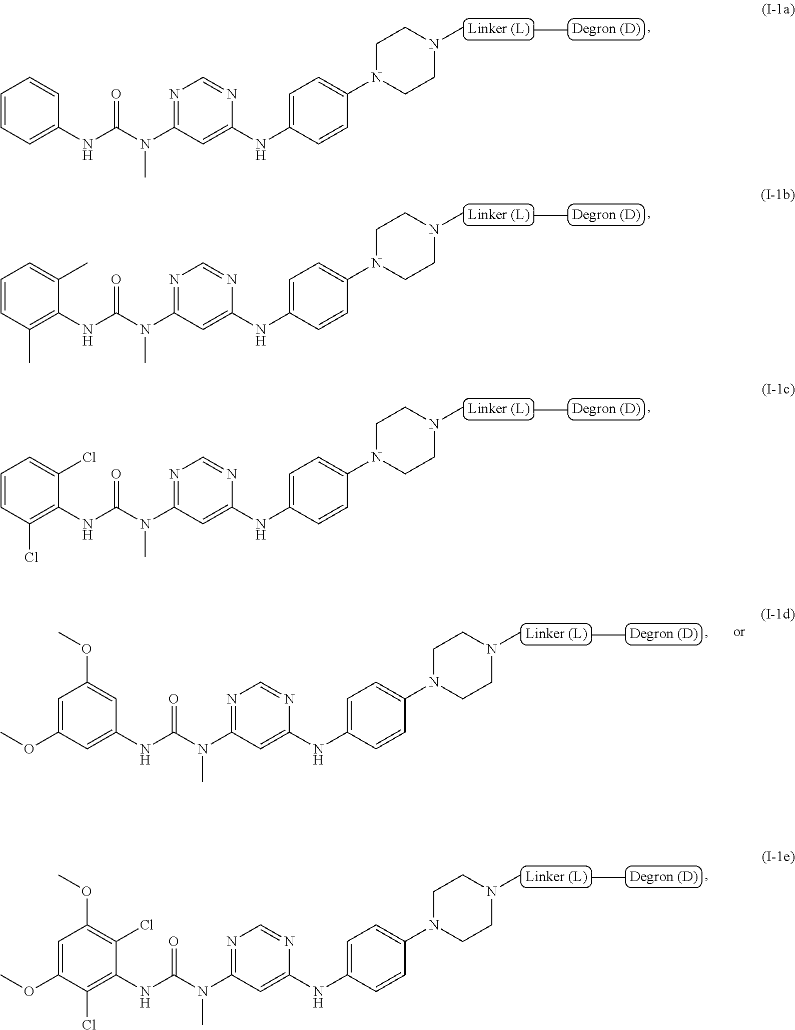

or a pharmaceutically acceptable salt or stereoisomer thereof.

In some embodiments, the targeting ligand has a structure represented by formula (TL-2):

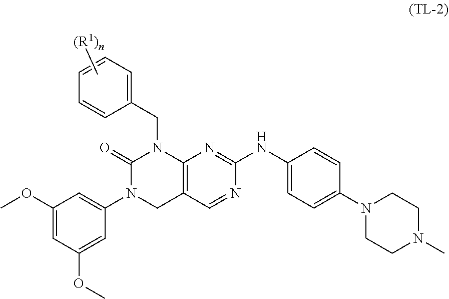

wherein:
R¹ is H or optionally substituted alkyl, optionally substituted alkoxy, optionally substituted amino, optionally substituted amido, carboxyl, acrylamide, optionally substituted carbocyclyl, or optionally substituted heterocyclyl; and n is an integer 0-4.

Thus, in some embodiments, the bispecific compounds of the present invention have a structure represented by formula (I-2):

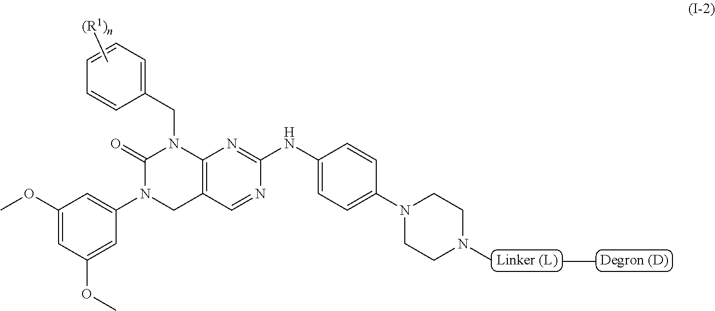

or a pharmaceutically acceptable salt or stereoisomer thereof.

In some embodiments, the bispecific compound of formula I-2 is represented by formula (I-2a):

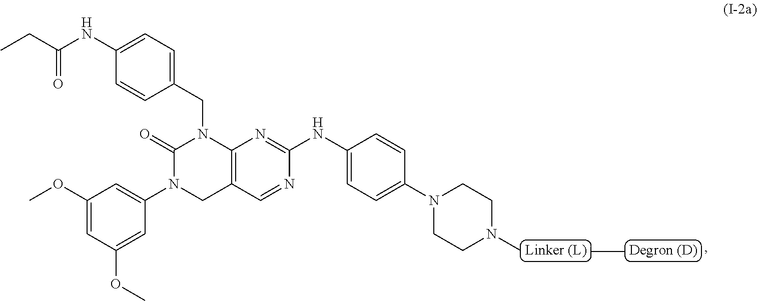

or a pharmaceutically acceptable salt or stereoisomer thereof.

In some embodiments, the targeting ligand has a structure represented by formula (TL-3):

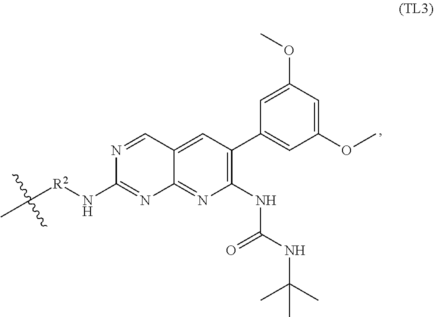

wherein R² is absent or represents

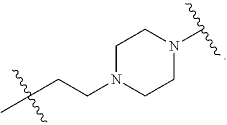 [SMILES: *CCN1CCN(*)CC1]

Thus, in some embodiments, the bispecific compounds of the present invention have a structure represented by formula (I-3a) or (L-3b):

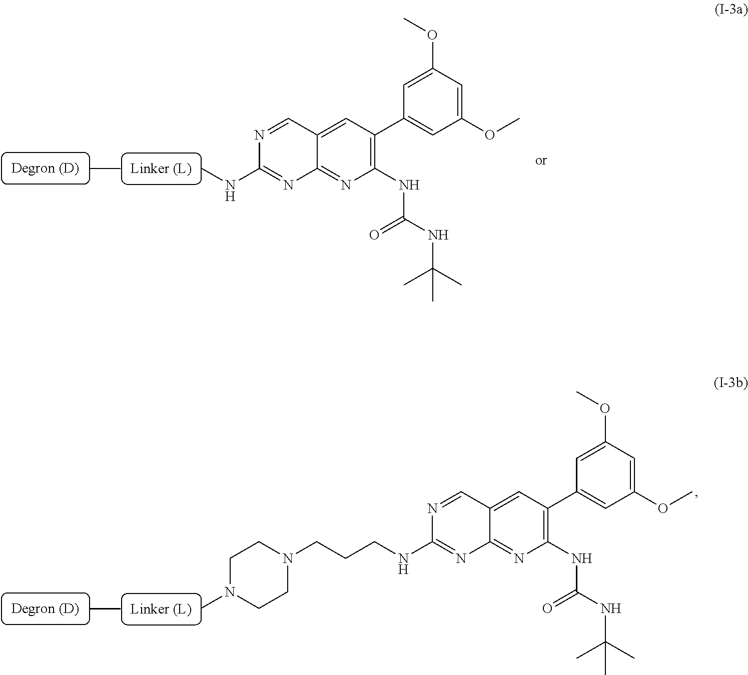

or a pharmaceutically acceptable salt or stereoisomer thereof.

In some embodiments, the targeting ligand has a structure represented by formula (TL-4):

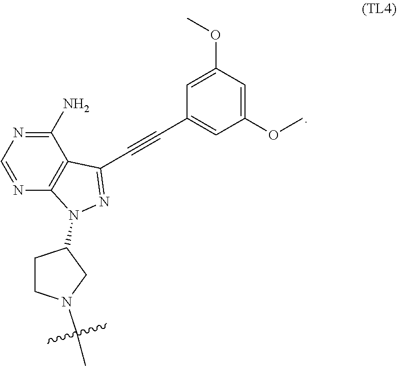

Thus, in some embodiments, the bispecific compounds of the present invention have a structure represented by formula (I-4):

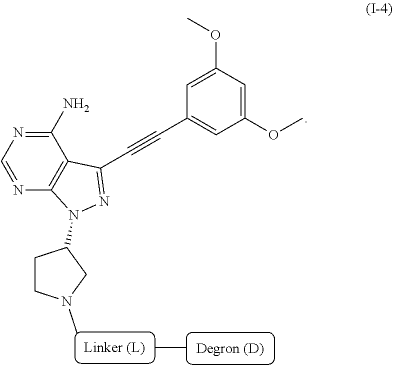

or a pharmaceutically acceptable salt or stereoisomer thereof.

Yet other moieties that may be useful as FGFR2 targeting ligands are described in U.S. Pat. Nos. 8,865,737 and 9,957,236, and U.S. Patent Application Publication Nos.: US2014/0378481, US2018/0155340, US2016/0009785, and US2015/0366866.

### Linkers

The linker (“L”) provides a covalent attachment the targeting ligand and the degron.

The structure of linker may not be critical, provided it does not substantially interfere with the activity of the targeting ligand or the degron.

In some embodiments, the linker may be an alkylene chain or a bivalent alkylene chain, either of which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

“Carbocyclene” refers to a bivalent carbocycle radical, which is optionally substituted.

“Heterocyclene” refers to a bivalent heterocyclyl radical which may be optionally substituted.

“Heteroarylene” refers to a bivalent heteroaryl radical which may be optionally substituted.

Representative examples of linkers that may be suitable for use in the present invention include alkylene chains:

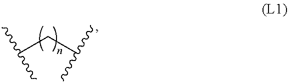

wherein n is an integer of 1-12 (“of” meaning inclusive), e.g., 1-12, 1-11, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10 and 1, 2, 3, 4, 5, 6, 7, 8, 9 and 10, examples of which include:

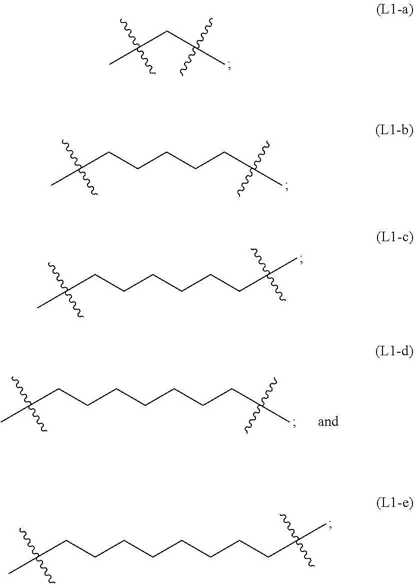

alkylene chains terminating in various functional groups (as described above), examples of which are as follows:

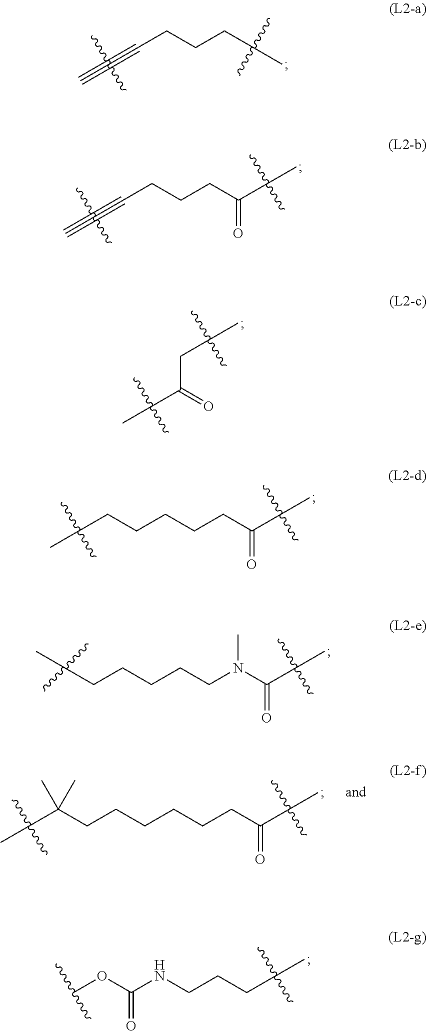

alkylene chains interrupted by various functional groups (as described above), examples of which are as follows:

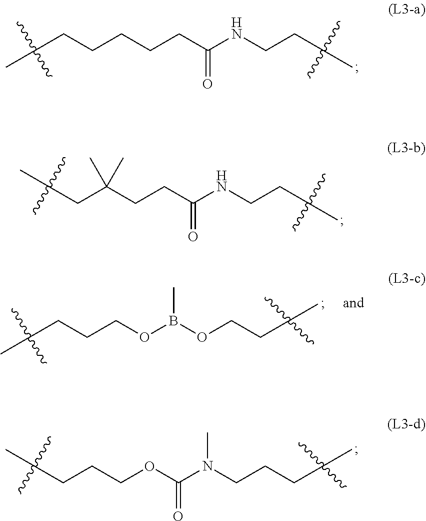 [SMILES: *CCCCCC(=O)NCC* ; *CCNC(=O)CCC(C)(C)C* ; *CCCOB(C)OCC* ; *CCCOC(=O)N(C)CCC*]

alkylene chains interrupted by or terminating with heterocyclene groups, e.g.,

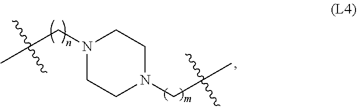

wherein m and n are independently integers of 0-10, examples of which include:

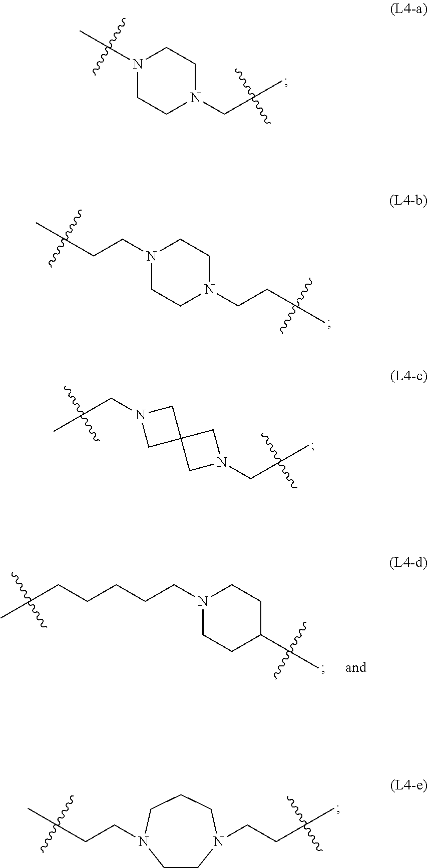 [SMILES: *CN1CCN(*)CC1 ; *CCN1CCN(CC*)CC1 ; *CN1CC2(C1)CN(C*)C2 ; *CCCCCN1CCC(*)CC1 ; *CCN1CCCN(CC*)CC1]

alkylene chains interrupted by amide, heterocyclene and/or aryl groups, examples of which include:

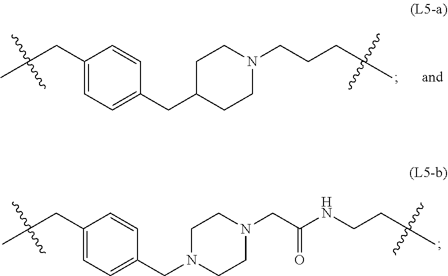 [SMILES: *CCCN1CCC(Cc2ccc(C*)cc2)CC1 ; *CCNC(=O)CN1CCN(Cc2ccc(C*)cc2)CC1]

alkylene chains interrupted by heterocyclene and aryl groups, and a heteroatom, examples of which include:

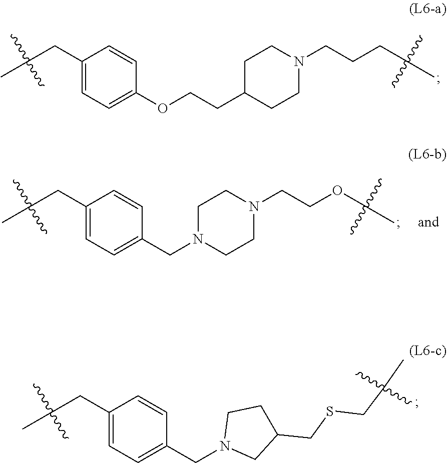 [SMILES: *CCCN1CCC(CCOc2ccc(C*)cc2)CC1 ; *Cc1ccc(CN2CCN(CCO*)CC2)cc1 ; *CSCC1CCN(Cc2ccc(C*)cc2)C1]

alkylene chains interrupted by and/or terminating in a heteroatom such as N, O or B, e.g.,

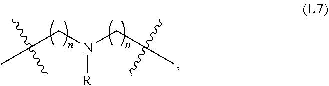

wherein each n is independently an integer of 1-10, e.g., 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10, and 1, 2, 3, 4, 5, 6, 7, 8, 9 and 10, and R is H or C1 to C4 alkyl, an example of which is

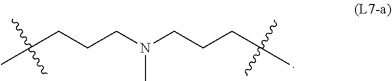 [SMILES: *CCCN(C)CCC*]

In some embodiments, the linker may be a polyethylene glycol chain which may terminate (at either or both termini) in at least one of —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃₋₁₂ carbocyclene, 3- to 12-membered heterocyclene, 5- to 12-membered heteroarylene or any combination thereof, wherein R′ is H or C₁-C₆ alkyl, wherein the one or both terminating groups may be the same or different.

Examples of linkers that include a polyethylene glycol chain include:

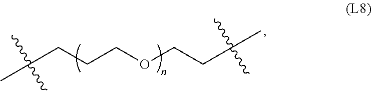

wherein n is an integer of 2-10, examples of which include:

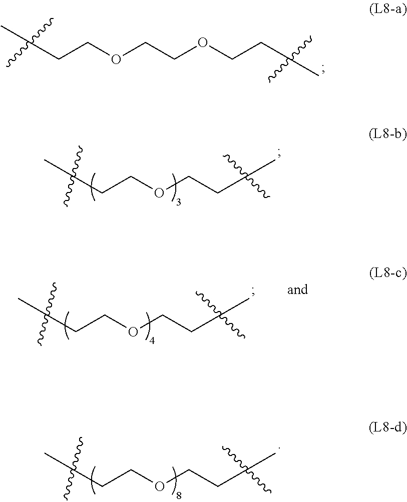

In some embodiments, the polyethylene glycol chain may terminate in a functional group, examples of which are as follows:

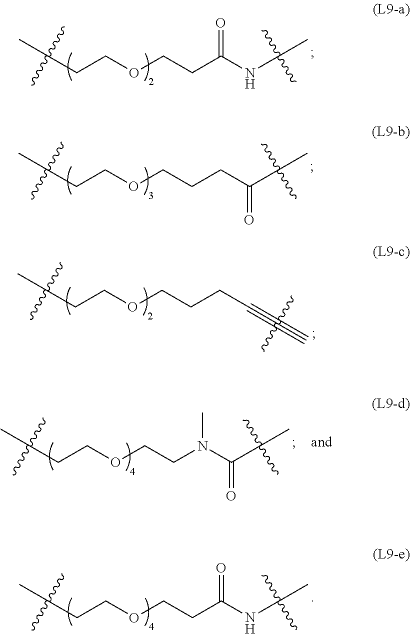

In some embodiments, the linker is represented by a structure selected from the group consisting of:

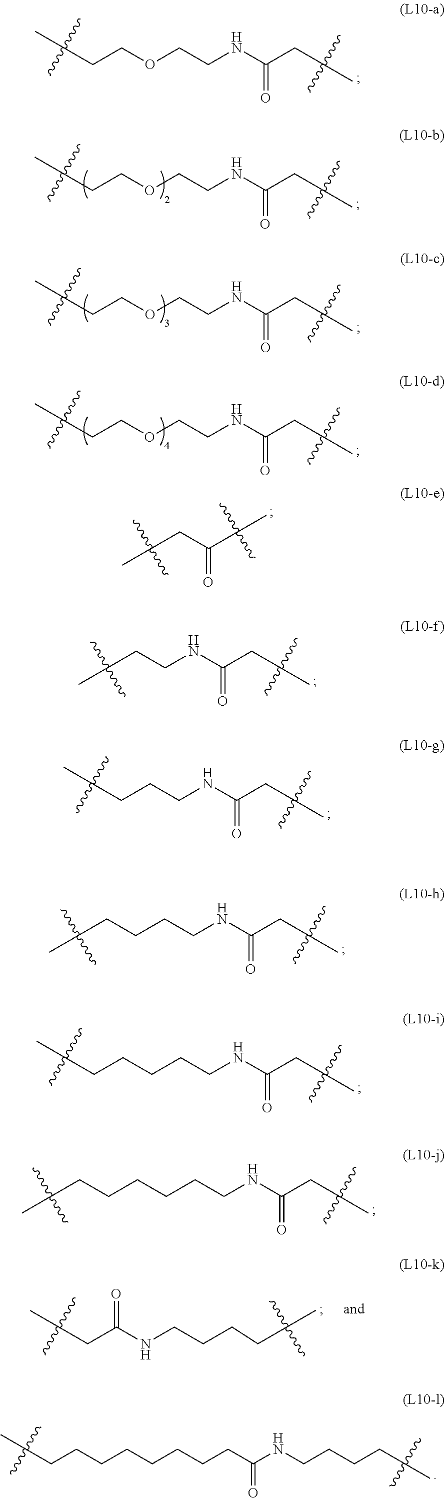

In some embodiments, bispecific compounds of the present invention may include an FGFR2 TL linked to a degron via a PEG linker that terminates in a functional group. Representative examples of bispecific compounds include:

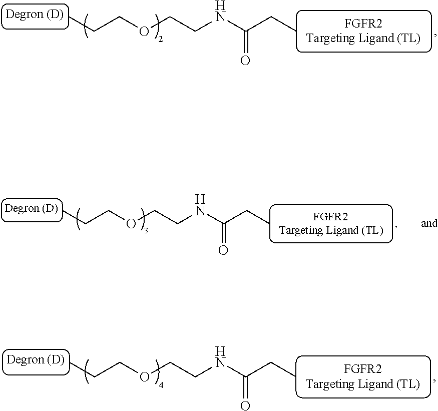

or a pharmaceutically acceptable salt or stereoisomer thereof.

In some embodiments, bispecific compounds of the present invention may include a TL linked to a degron via an alkylene linker that may be interrupted by and/or terminating in a cyclic or non-cyclic group (e.g., an amide group), or one or more heteroatoms. Representative examples of bispecific compounds include:

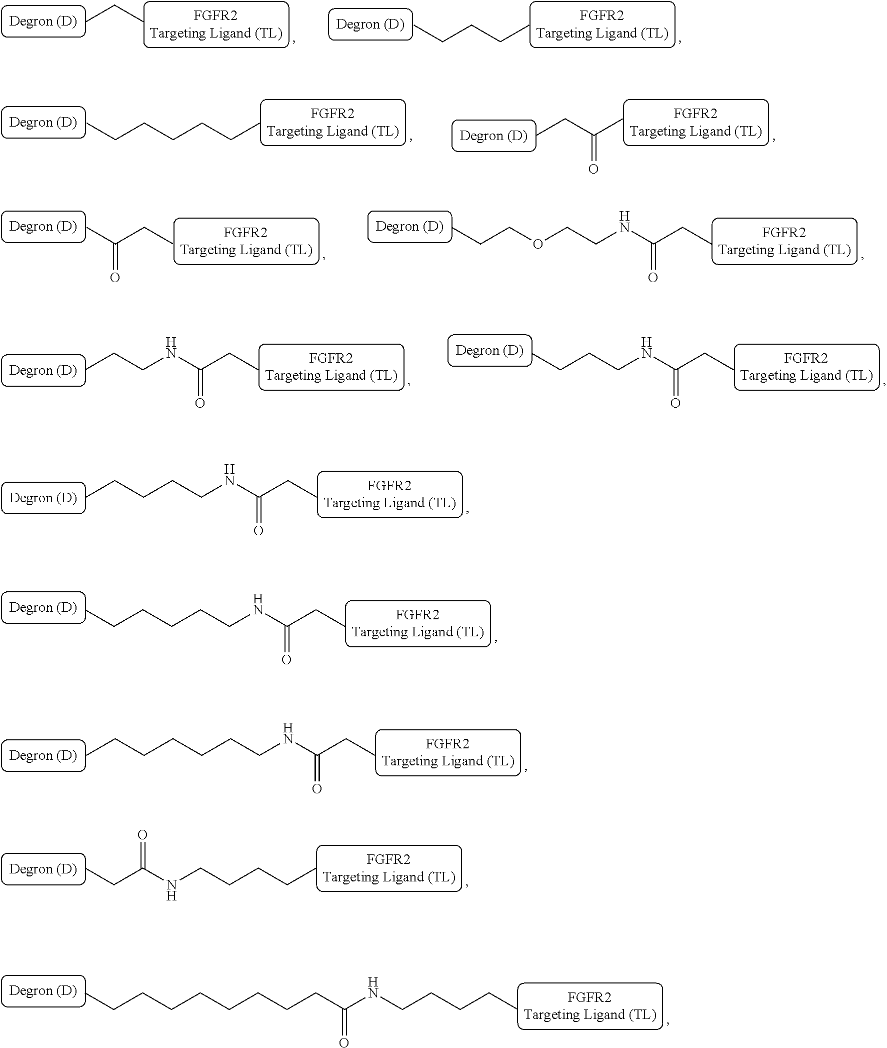

or a pharmaceutically acceptable salt or stereoisomer thereof.

In some embodiments, the bispecific compounds of the present invention are represented by any one of the following structures (with the Degron shown generically):

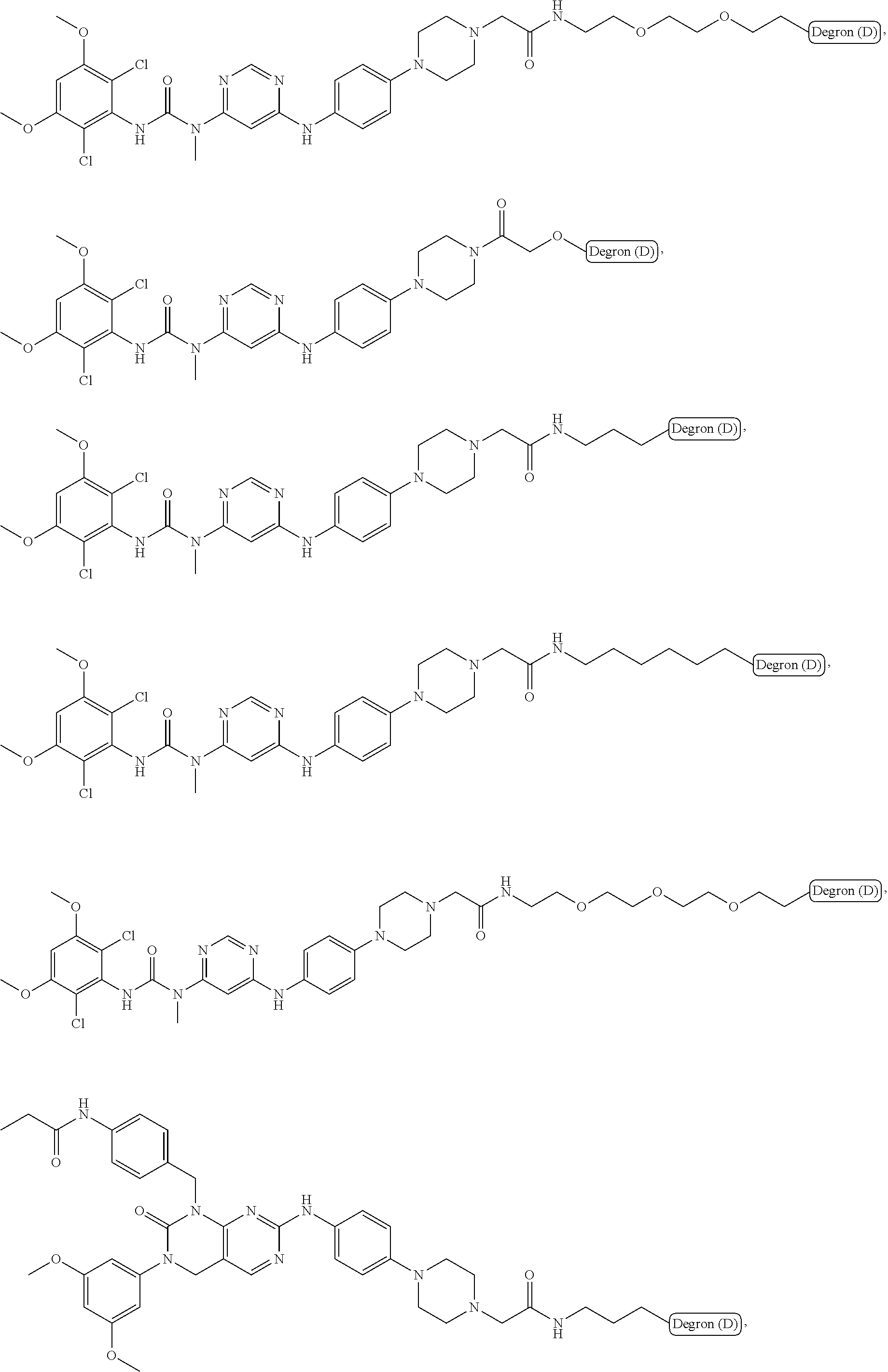

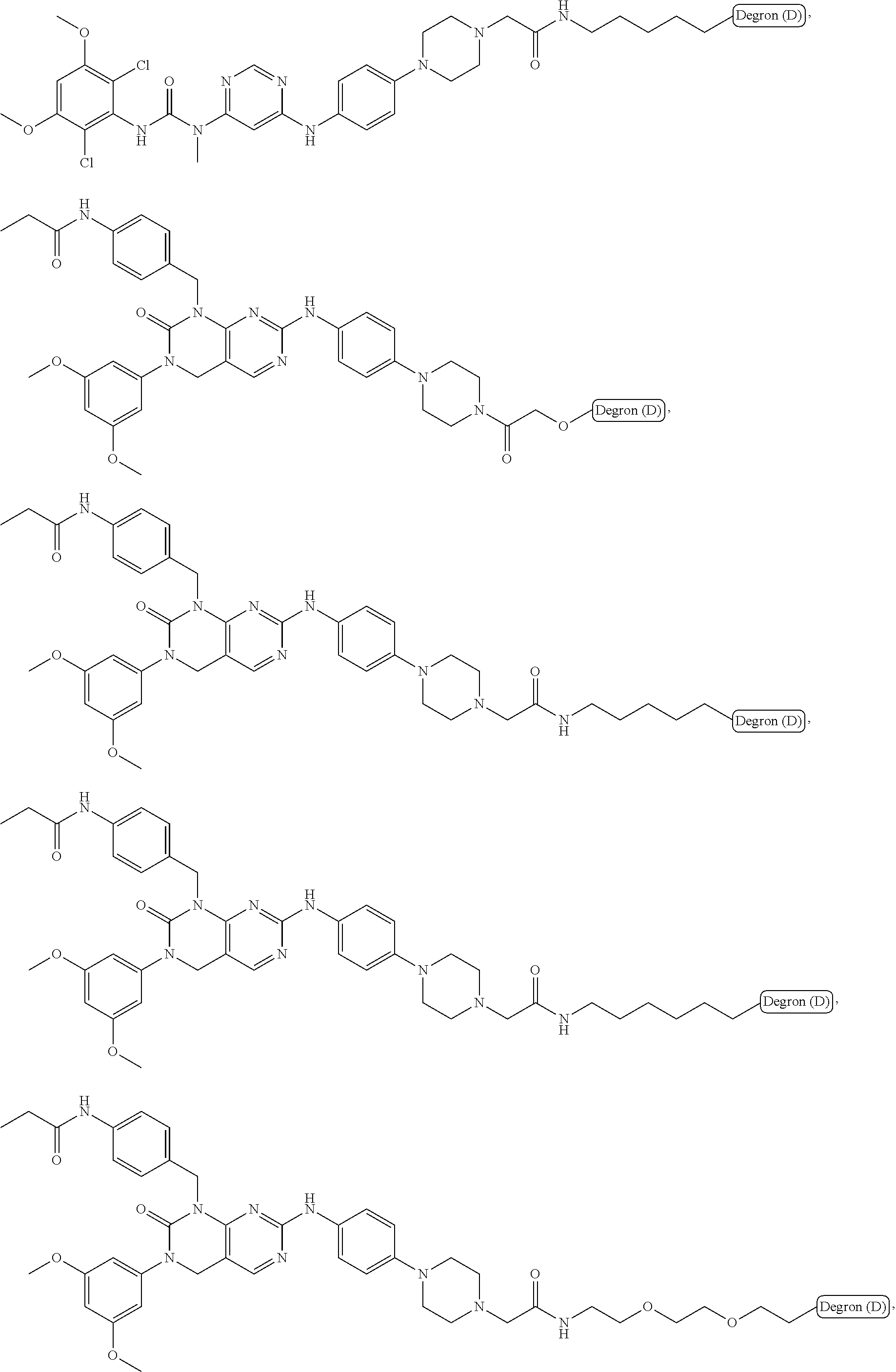

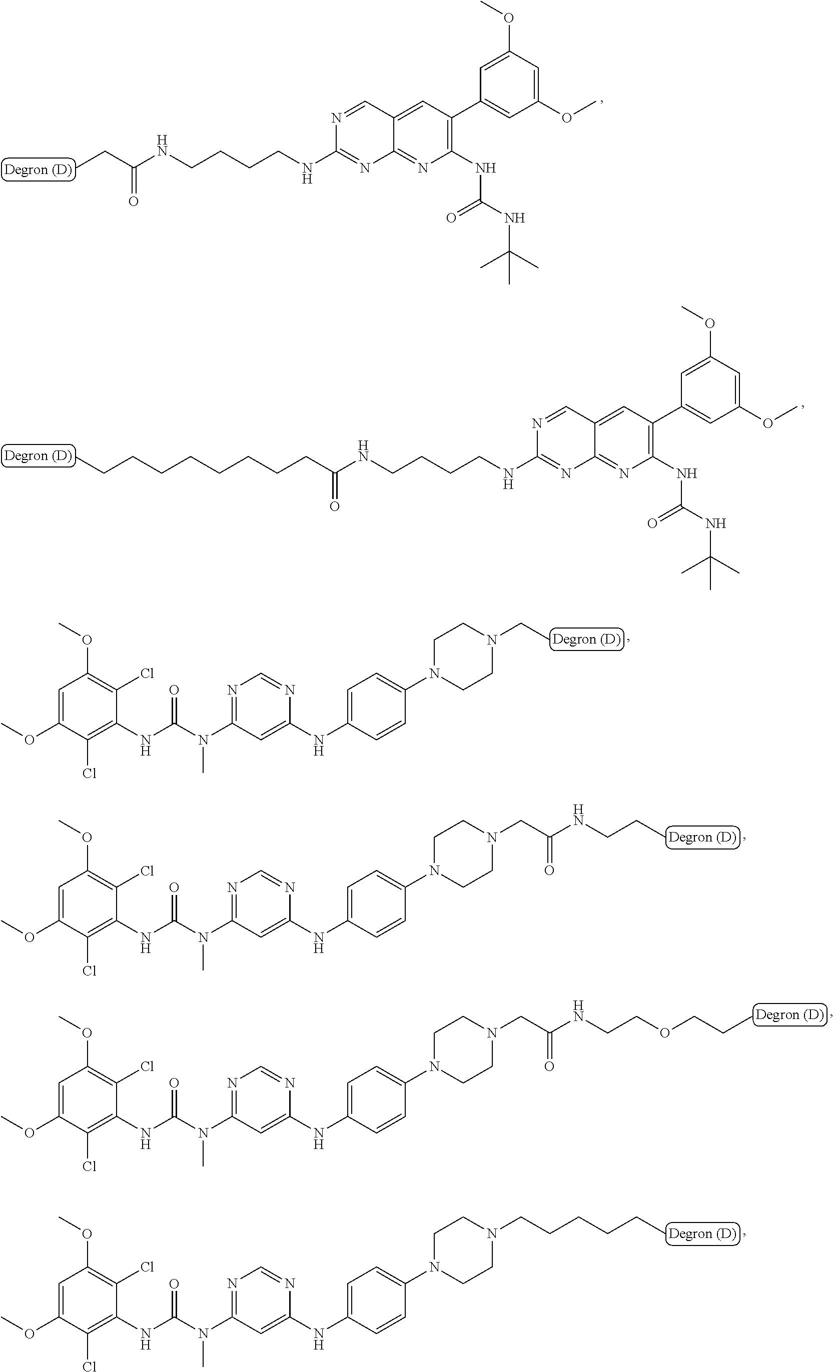

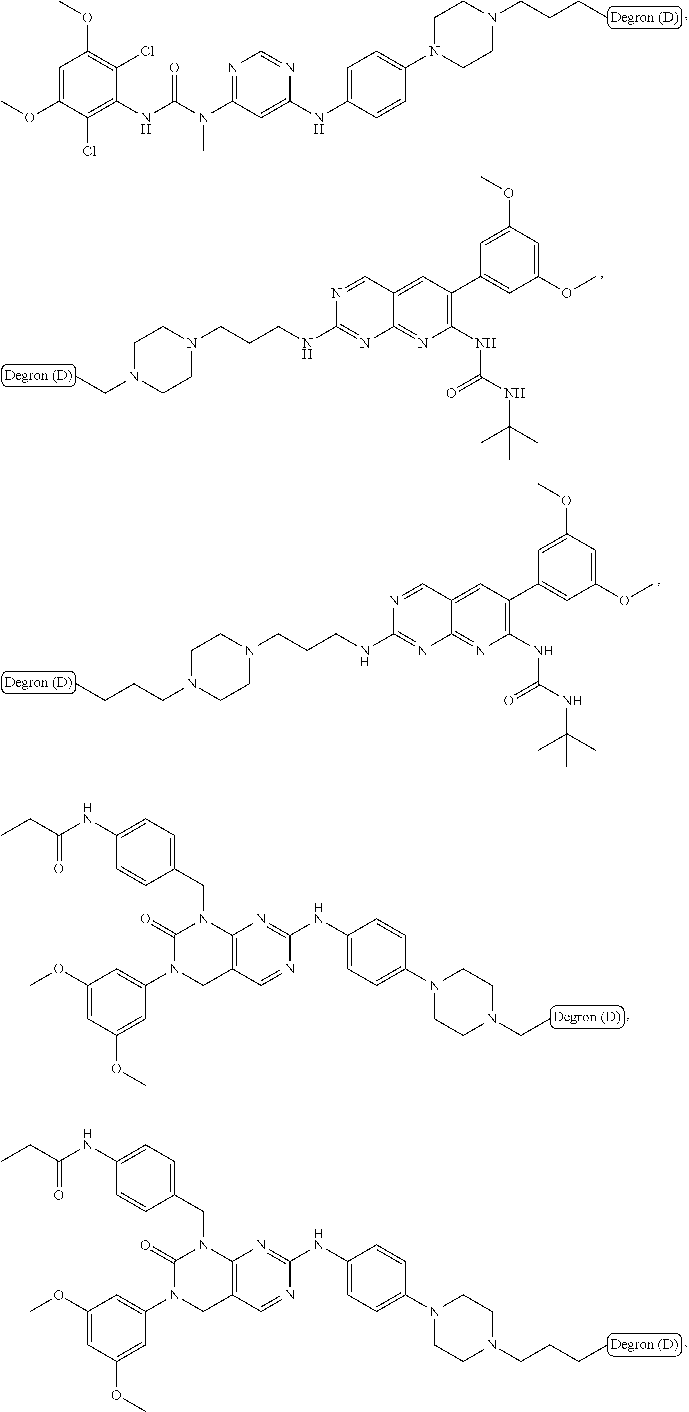

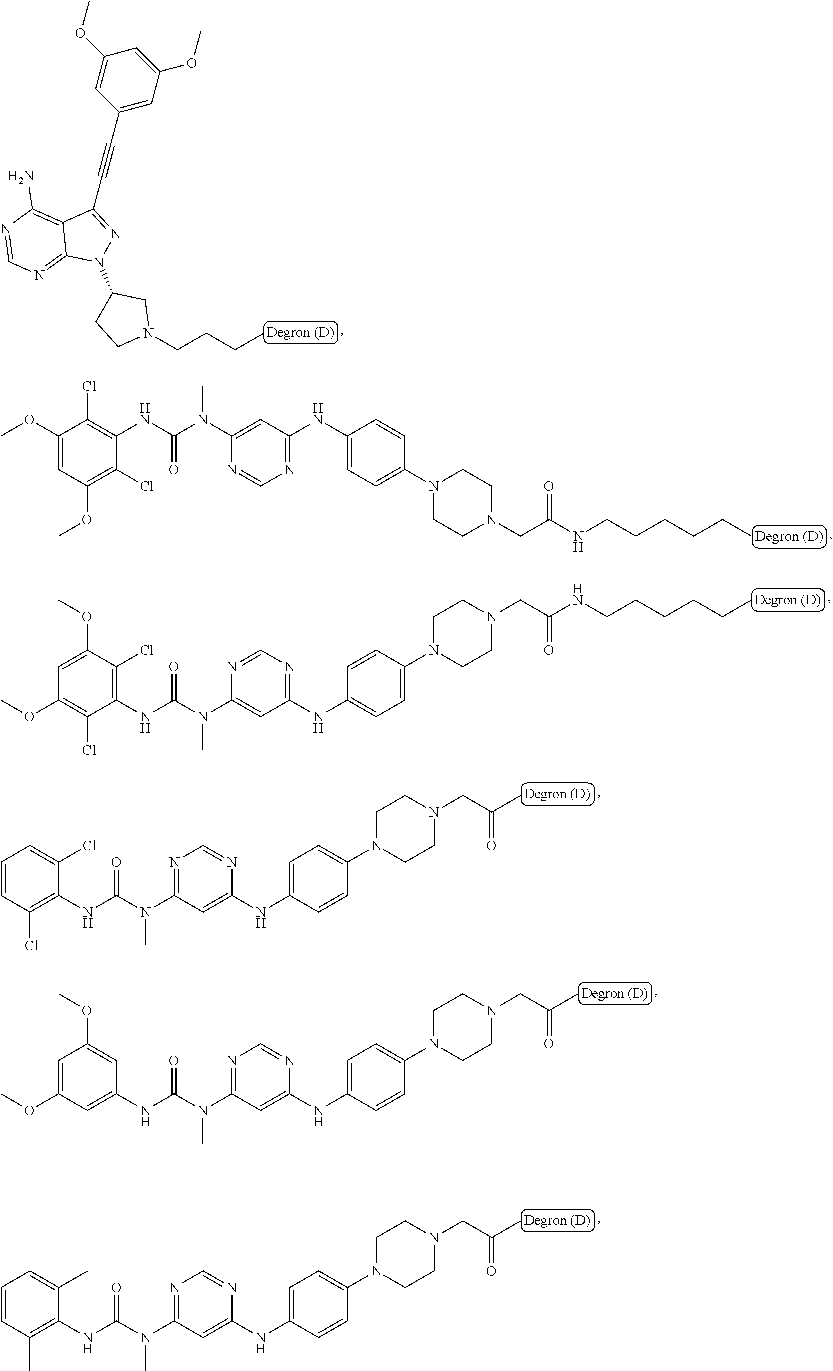

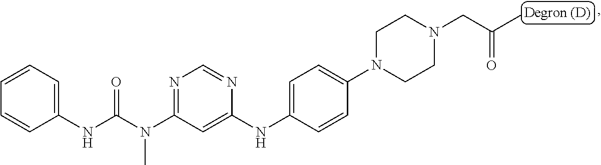 [SMILES: CC(=O)CN1CCN(c2ccc(Nc3cc(N(C)C(=O)Nc4ccccc4)ncn3)cc2)CC1]

or a pharmaceutically acceptable salt or stereoisomer thereof.

### Degrons

The Ubiquitin-Proteasome Pathway (UPP) is a critical cellular pathway that regulates key regulator proteins and degrades misfolded or abnormal proteins. UPP is central to multiple cellular processes. The covalent attachment of ubiquitin to specific protein substrates is achieved through the action of E3 ubiquitin ligases. These ligases include over 500 different proteins and are categorized into multiple classes defined by the structural element of their E3 functional activity.

In some embodiments, the degron binds the E3 ubiquitin ligase which is cereblon and is represented by a structure selected from the group consisting of

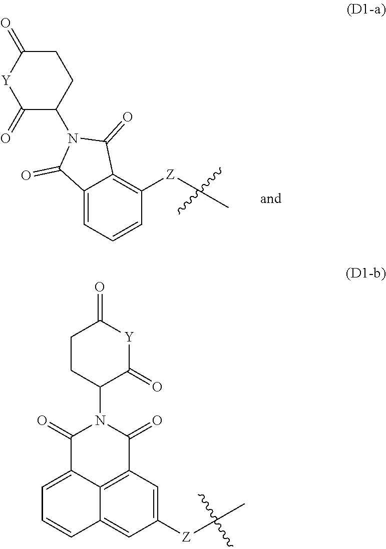

wherein

### Y is NH, NMe, or O; and

### Z is NH, O, or C≡.

Thus, in some embodiments, the bispecific compounds of this invention are represented by a formula selected from the group consisting of

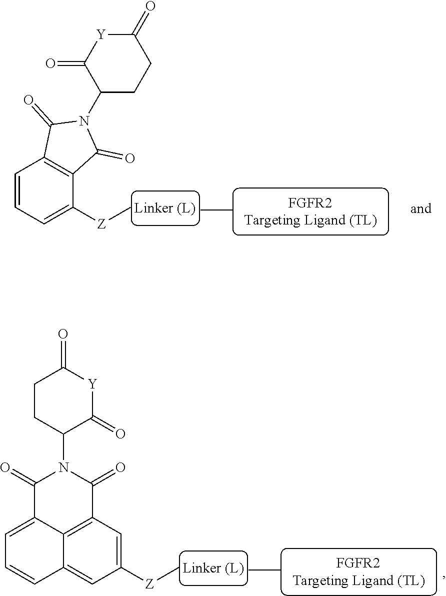

wherein

### Y is NH, NMe, or O; and

### Z is NH, O, or C≡;

or a pharmaceutically acceptable salt, or stereoisomer thereof.

Yet other degrons that bind cereblon and which may be suitable for use in the present invention are disclosed in U.S. Pat. No. 9,770,512, and U.S. Patent Application Publication Nos. 2018/0015087, 2018/0009779, 2016/0243247, 2016/0235731, 2016/0235730, and 2016/0176916, and International Patent Publications WO 2017/197055, WO 2017/197051, WO 2017/197036, WO 2017/197056 and WO 2017/197046.

In some embodiments, the E3 ubiquitin ligase that is bound by the degron is the von Hippel-Lindau (VHL) tumor suppressor. See, Iwai, et al., Proc. Nat'l. Acad. Sci. USA 96:12436-41 (1999).

In some embodiments, the degrons that bind VHL are represented by any one of the following-structures:

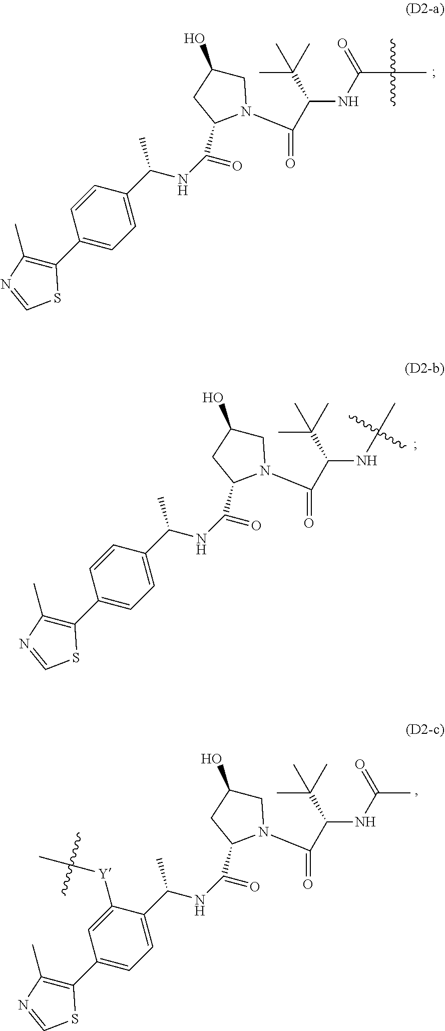

wherein Y′ is a bond, N, O or C;

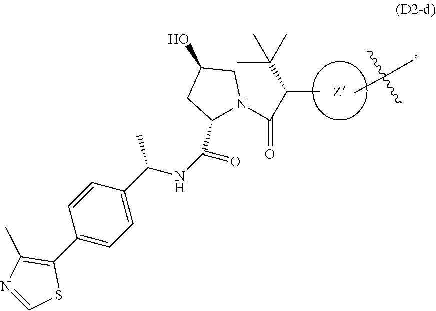

wherein Z′ is a cyclic group, which in some embodiments is a C5-6 carbocyclic or heterocyclic group, and

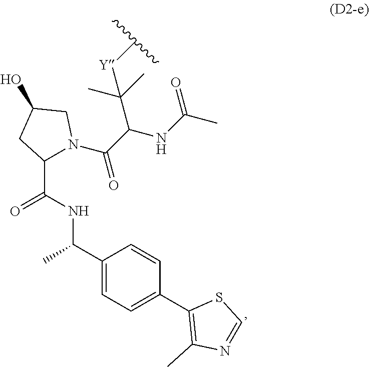

wherein Y″ is a bond, CH₂, NH, NMe, O, or S, or a stereoisomer thereof. In certain embodiments, the heterocyclic group is

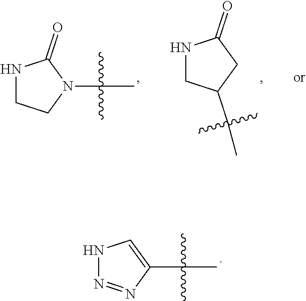 [SMILES: *N1CCNC1=O ; *C1CNC(=O)C1 ; *c1c[nH]nn1]

In some embodiments, the inventive bispecific compounds may be represented by any one of the following structures:

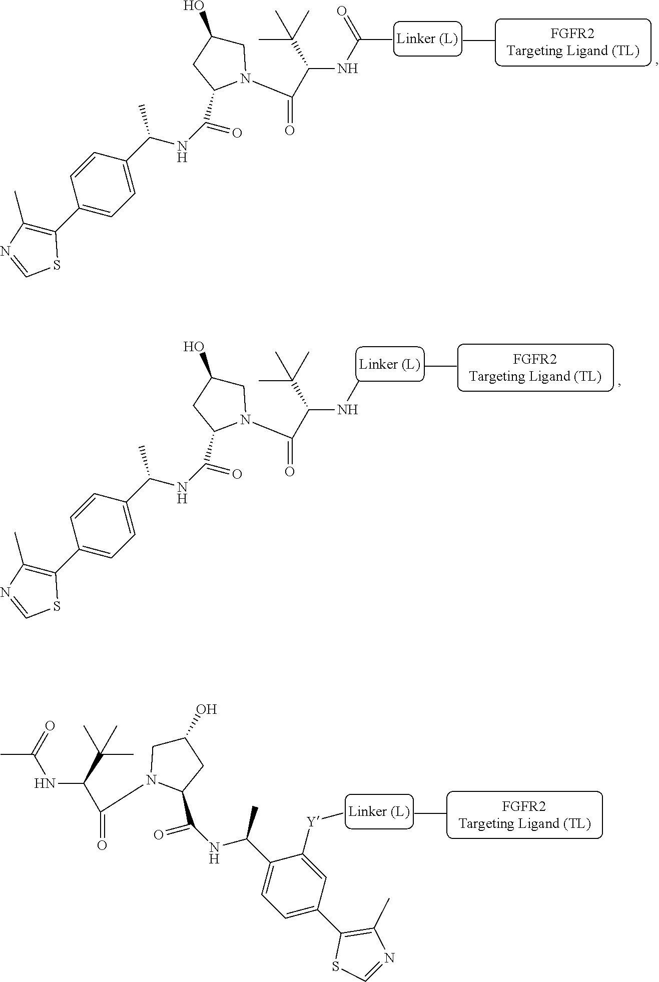

wherein Y′ is a bond, NH, O or CH₂,

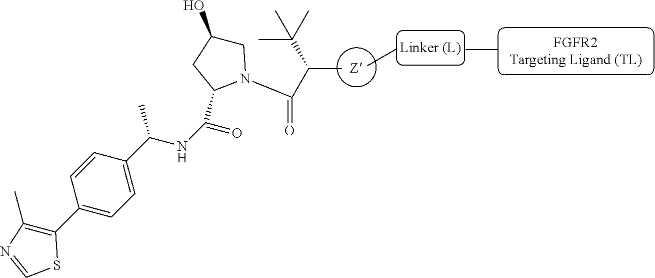

wherein Z′ is a cyclic group,

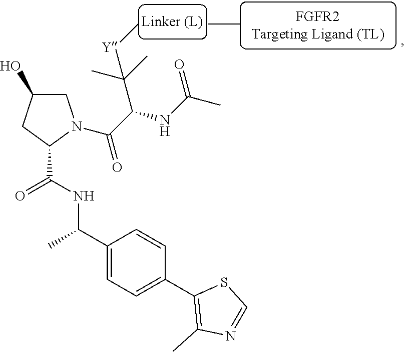

or a pharmaceutically acceptable salt or stereoisomer thereof.

In some embodiments, Z′ is phenyl, pyrrolyl, furanyl, thiophenyl, pyrazolyl, imidazolyl, oxazolyl, thiazolyl, pyridinyl, pyridazinyl, pyrimidinyl, benzofuranyl, benzothiophenyl, indolyl, quinolinyl, or isoquinolinyl. In certain embodiments, Z′ is

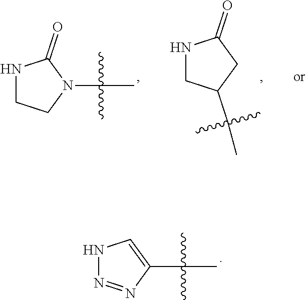 [SMILES: *N1CCNC1=O ; *C1CNC(=O)C1 ; *c1c[nH]nn1]

Yet other degrons that bind VHL and which may be suitable for use in the present invention are disclosed in U.S. Patent Application Publication 2017/0121321 A1.

In some embodiments, the E3 ubiquitin ligase that is bound by the degron is an inhibitor of apoptosis protein (IAP). Representative examples of degrons that bind IAP and may be suitable for use in the present invention are represented by any one of the following structures:

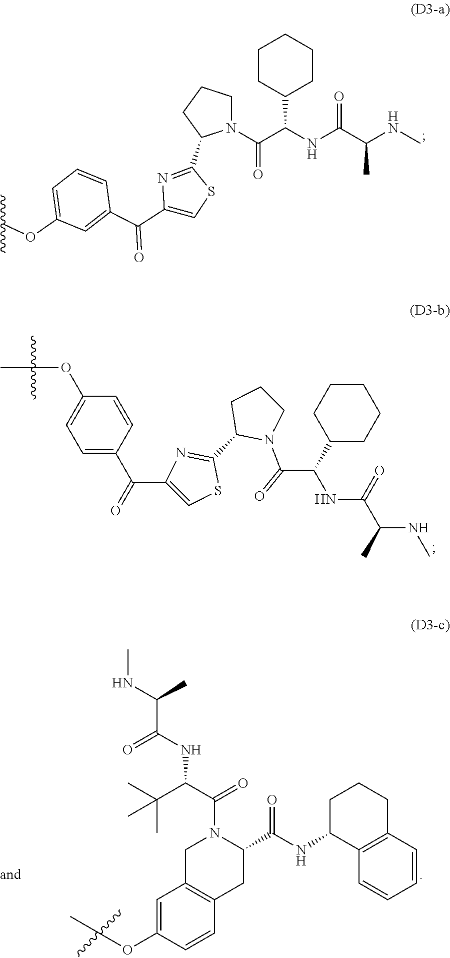 [SMILES: *Oc1cccc(C(=O)c2csc([C@@H]3CCCN3C(=O)[C@@H](NC(=O)[C@H](C)NC)C3CCCCC3)n2)c1 ; *Oc1ccc(C(=O)c2csc([C@@H]3CCCN3C(=O)[C@@H](NC(=O)[C@H](C)NC)C3CCCCC3)n2)cc1 ; *Oc1ccc2c(c1)CN(C(=O)[C@@H](NC(=O)[C@H](C)NC)C(C)(C)C)[C@H](C(=O)N[C@@H]1CCCc3ccccc31)C2]

Thus, in some embodiments, the bispecific compounds of the present invention are represented by any one of the following structures:

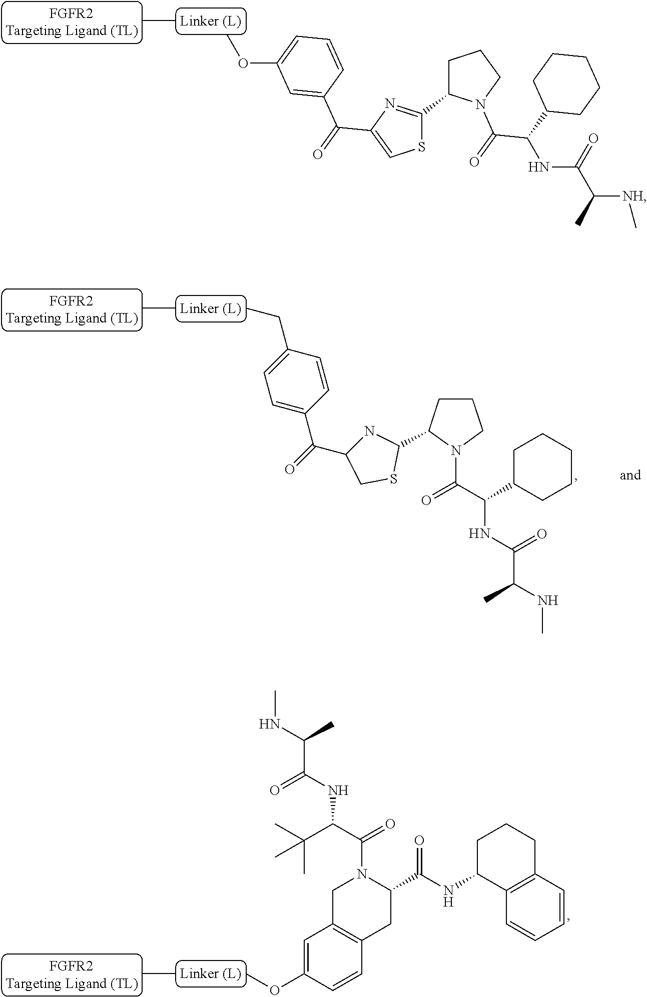

or a pharmaceutically acceptable salt or stereoisomer thereof.

Yet other degrons that bind IAPs and which may be suitable for use as degrons in the present invention are disclosed in International Patent Application Publications WO 2008128171, WO 2008/016893, WO 2014/060768, WO 2014/060767, and WO 15092420.

Thus, in some embodiments, the bispecific compounds of the present invention are represented by any structures generated by the combination of structures TL-1 to TL-4, L1 to L10, and the structures of the degrons described herein, including D1 to D3, or a pharmaceutically acceptable salt or stereoisomer thereof.

In some embodiments, the bispecific compounds of the present invention have the following structures:

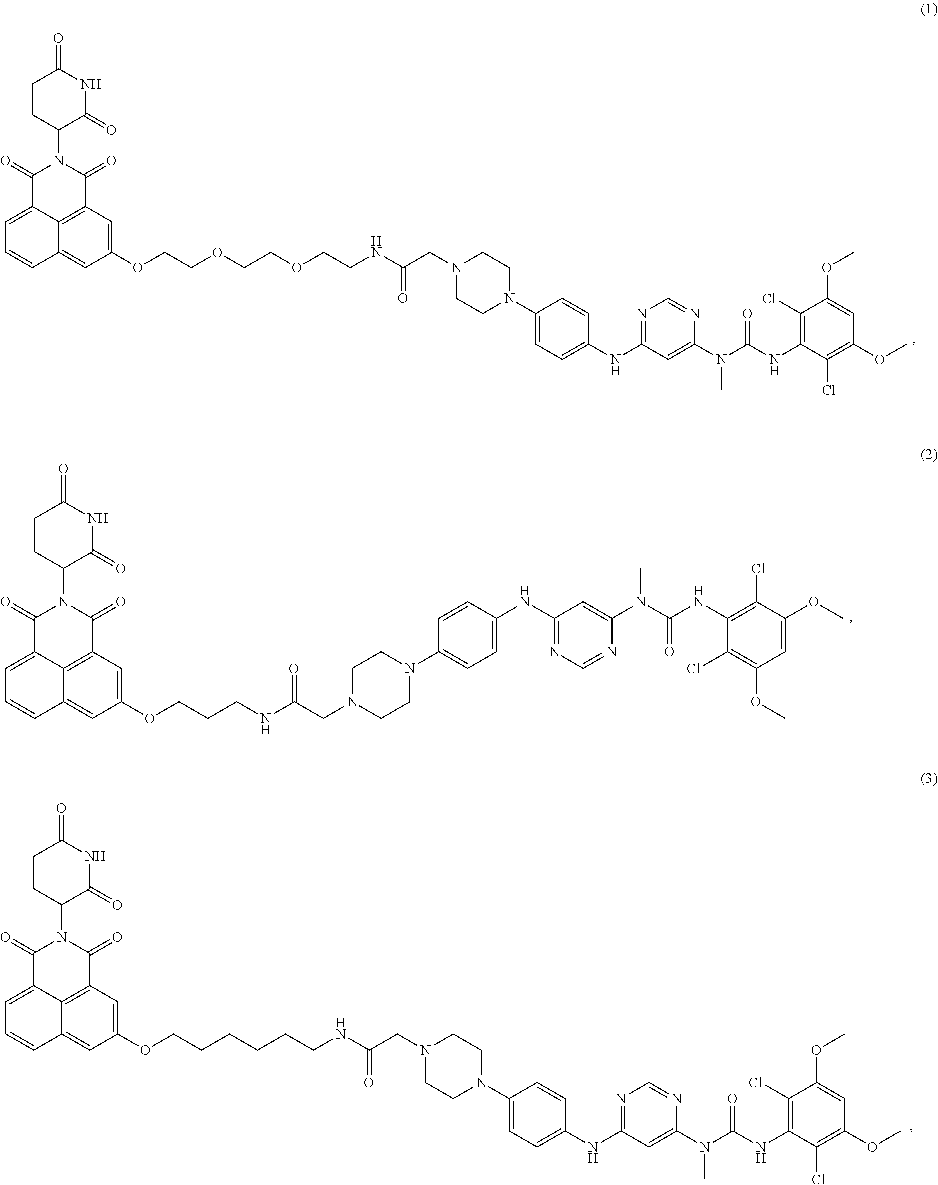 [SMILES: COc1cc(OC)c(Cl)c(NC(=O)N(C)c2cc(Nc3ccc(N4CCN(CC(=O)NCCOCCOCCOc5cc6c7c(cccc7c5)C(=O)N(C5CCC(=O)NC5=O)C6=O)CC4)cc3)ncn2)c1Cl ; COc1cc(OC)c(Cl)c(NC(=O)N(C)c2cc(Nc3ccc(N4CCN(CC(=O)NCCCOc5cc6c7c(cccc7c5)C(=O)N(C5CCC(=O)NC5=O)C6=O)CC4)cc3)ncn2)c1Cl ; COc1cc(OC)c(Cl)c(NC(=O)N(C)c2cc(Nc3ccc(N4CCN(CC(=O)NCCCCCCOc5cc6c7c(cccc7c5)C(=O)N(C5CCC(=O)NC5=O)C6=O)CC4)cc3)ncn2)c1Cl]

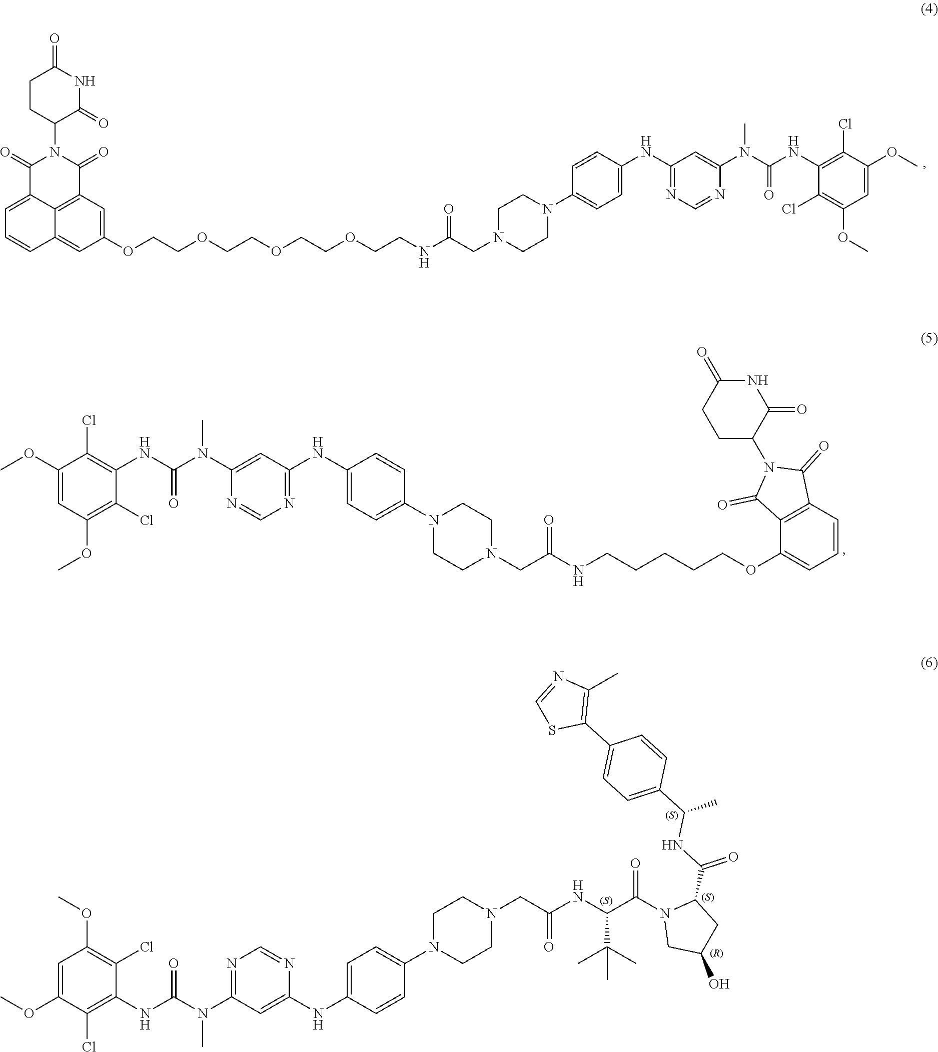

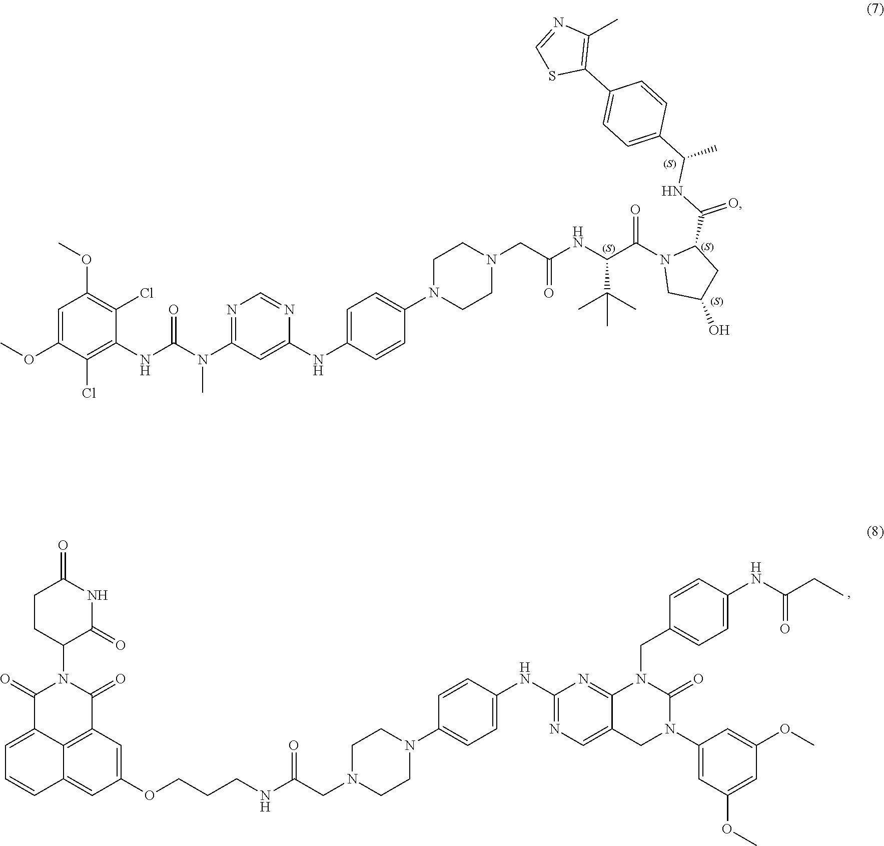

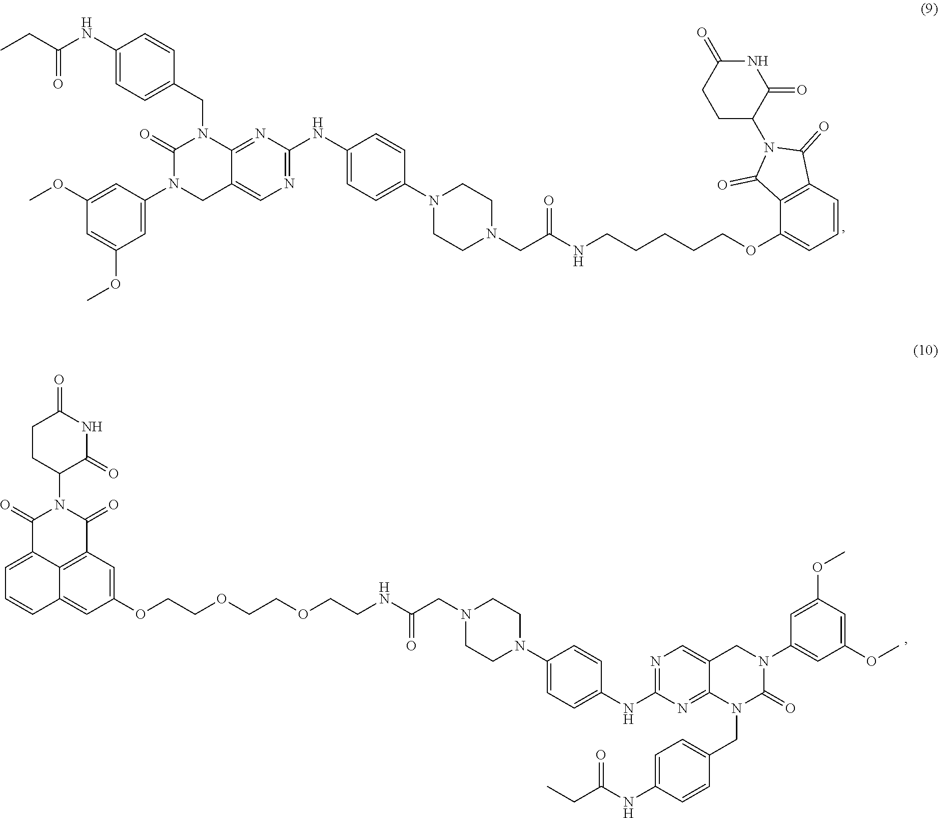 [SMILES: CCC(=O)Nc1ccc(CN2C(=O)N(c3cc(OC)cc(OC)c3)Cc3cnc(Nc4ccc(N5CCN(CC(=O)NCCCCCOc6cccc7c6C(=O)N(C6CCC(=O)NC6=O)C7=O)CC5)cc4)nc32)cc1 ; CCC(=O)Nc1ccc(CN2C(=O)N(c3cc(OC)cc(OC)c3)Cc3cnc(Nc4ccc(N5CCN(CC(=O)NCCOCCOCCOc6cc7c8c(cccc8c6)C(=O)N(C6CCC(=O)NC6=O)C7=O)CC5)cc4)nc32)cc1]

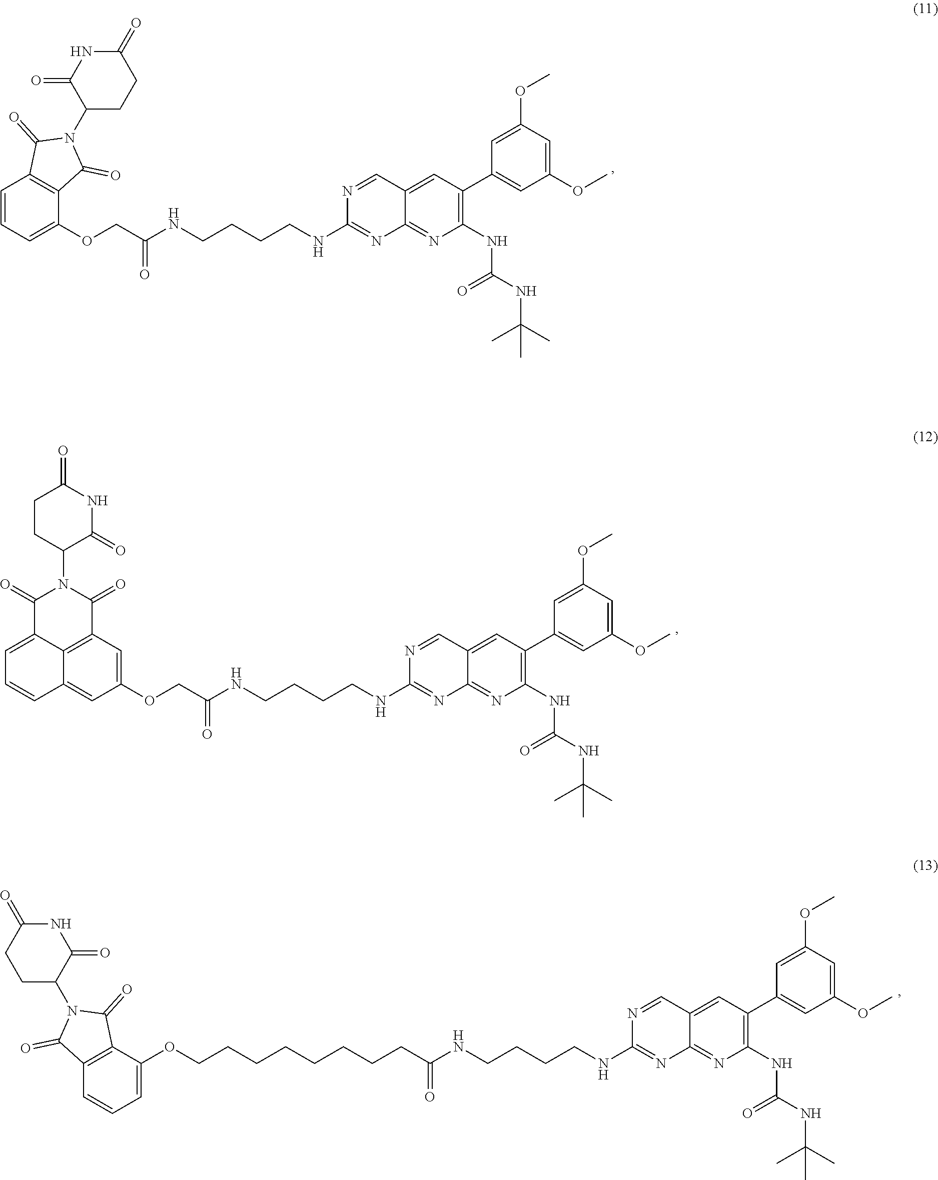 [SMILES: COc1cc(OC)cc(-c2cc3cnc(NCCCCNC(=O)COc4cccc5c4C(=O)N(C4CCC(=O)NC4=O)C5=O)nc3nc2NC(=O)NC(C)(C)C)c1 ; COc1cc(OC)cc(-c2cc3cnc(NCCCCNC(=O)COc4cc5c6c(cccc6c4)C(=O)N(C4CCC(=O)NC4=O)C5=O)nc3nc2NC(=O)NC(C)(C)C)c1 ; COc1cc(OC)cc(-c2cc3cnc(NCCCCNC(=O)CCCCCCCCOc4cccc5c4C(=O)N(C4CCC(=O)NC4=O)C5=O)nc3nc2NC(=O)NC(C)(C)C)c1]

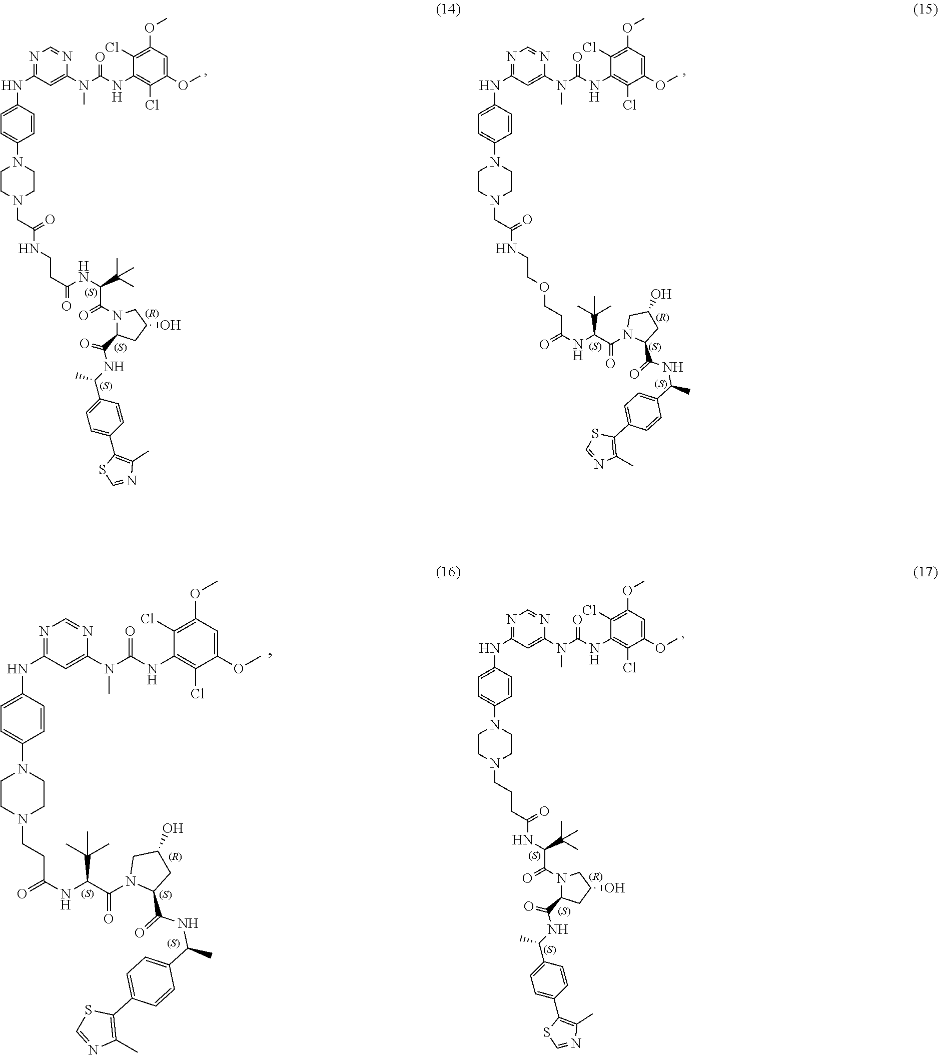

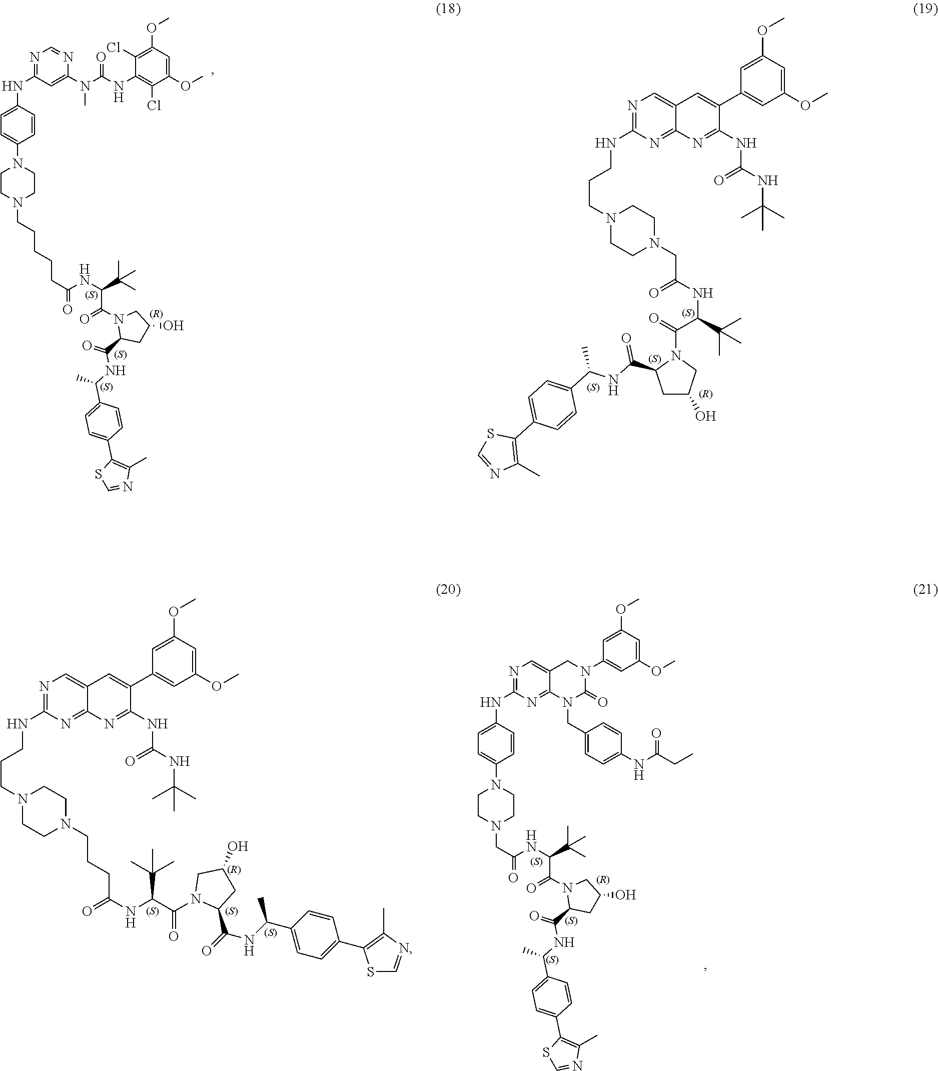

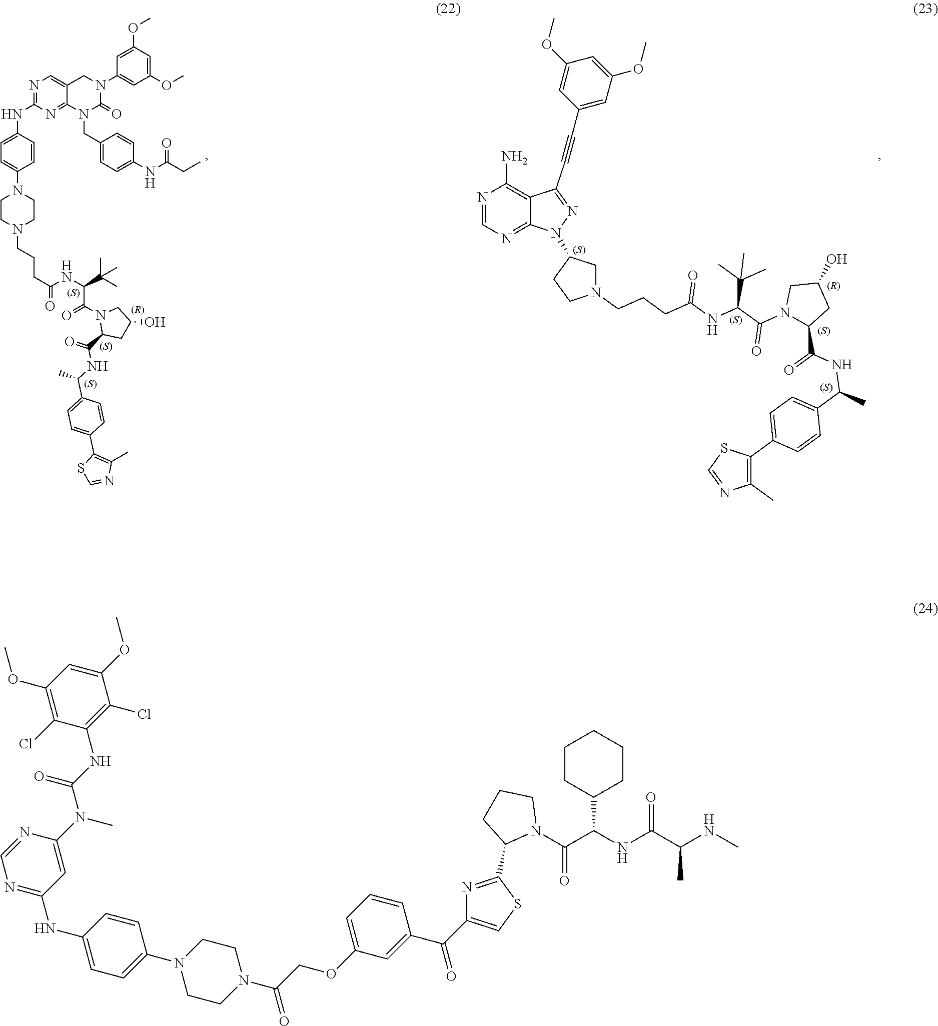

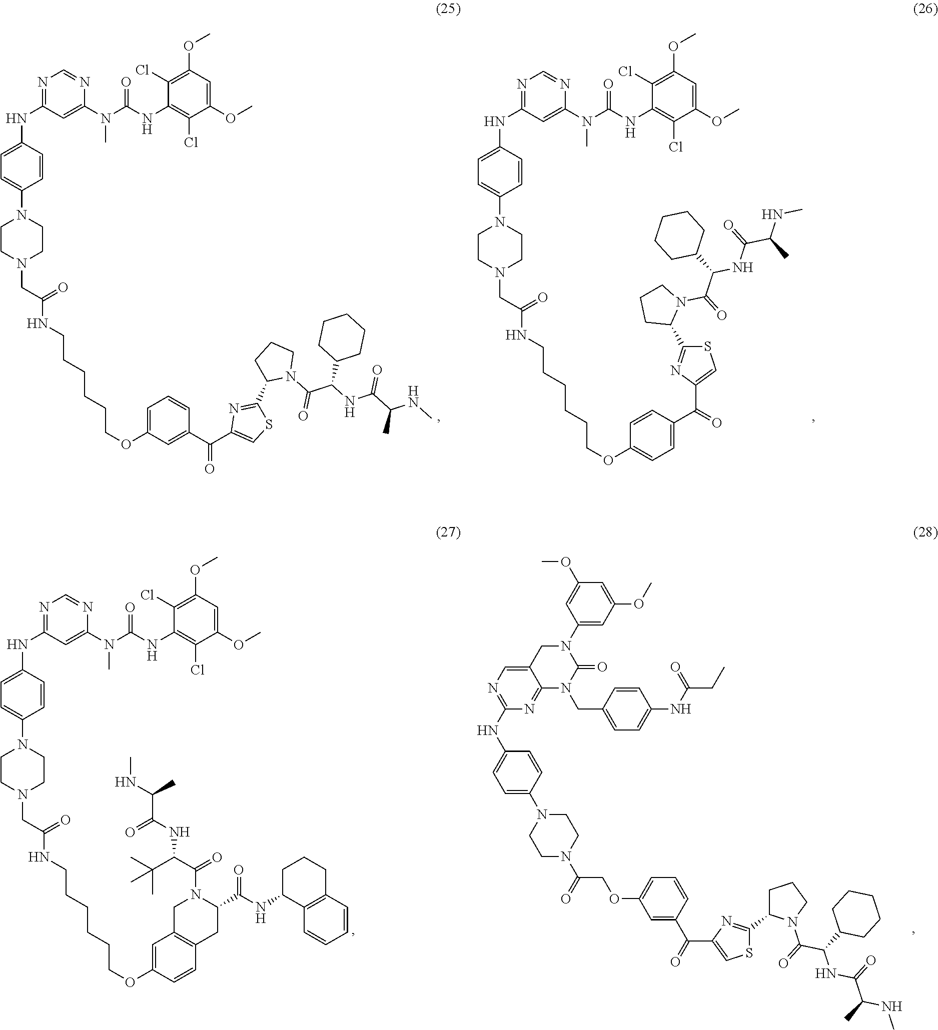

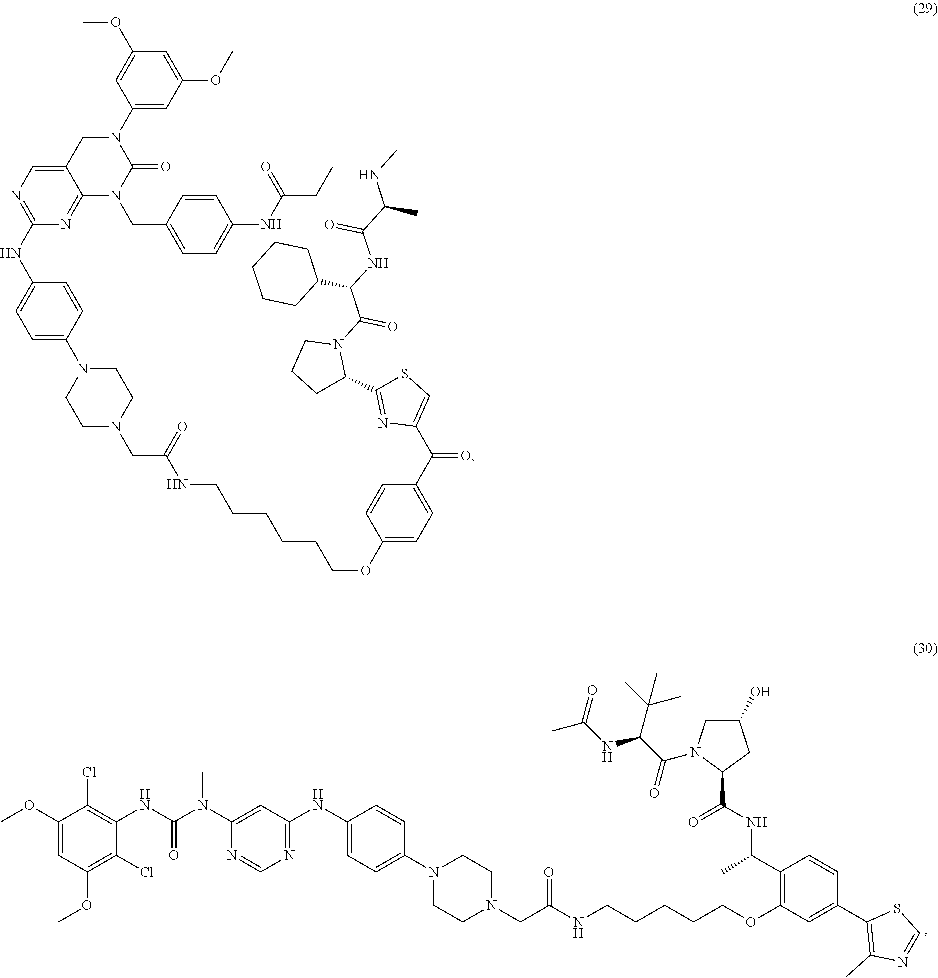 [SMILES: CCC(=O)Nc1ccc(CN2C(=O)N(c3cc(OC)cc(OC)c3)Cc3cnc(Nc4ccc(N5CCN(CC(=O)NCCCCCCOc6ccc(C(=O)c7csc([C@@H]8CCCN8C(=O)[C@@H](NC(=O)[C@H](C)NC)C8CCCCC8)n7)cc6)CC5)cc4)nc32)cc1 ; COc1cc(OC)c(Cl)c(NC(=O)N(C)c2cc(Nc3ccc(N4CCN(CC(=O)NCCCCCOc5cc(-c6scnc6C)ccc5[C@H](C)NC(=O)[C@@H]5C[C@@H](O)CN5C(=O)[C@@H](NC(C)=O)C(C)(C)C)CC4)cc3)ncn2)c1Cl]

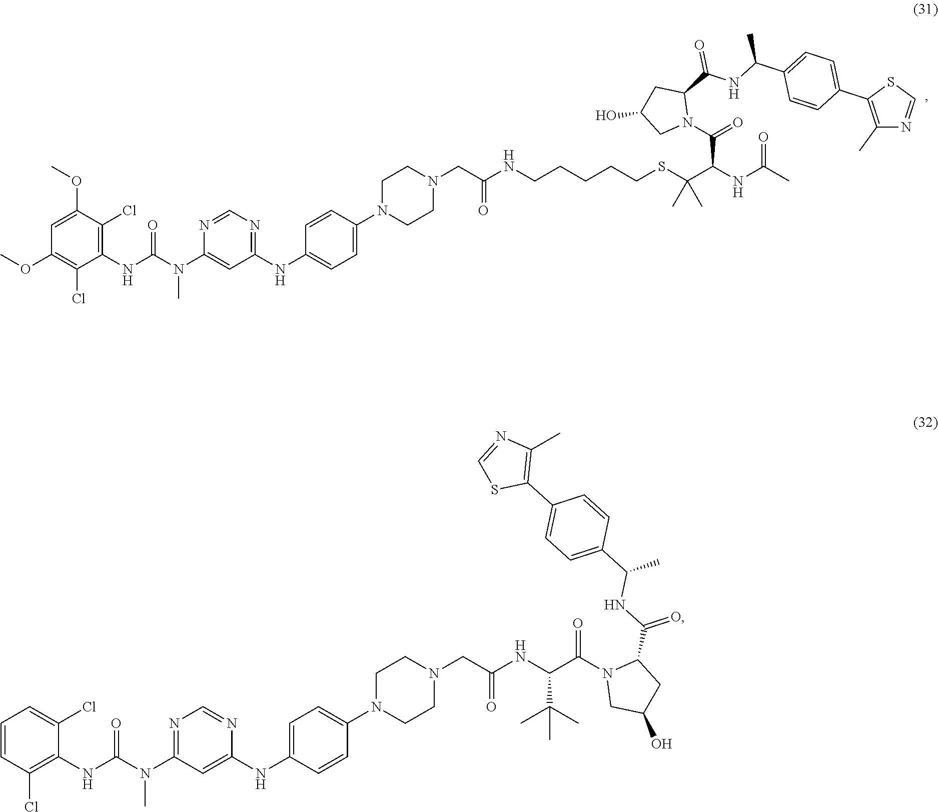 [SMILES: COc1cc(OC)c(Cl)c(NC(=O)N(C)c2cc(Nc3ccc(N4CCN(CC(=O)NCCCCCSC(C)(C)[C@H](NC(C)=O)C(=O)N5C[C@H](O)C[C@H]5C(=O)N[C@@H](C)c5ccc(-c6scnc6C)cc5)CC4)cc3)ncn2)c1Cl ; Cc1ncsc1-c1ccc([C@H](C)NC(=O)[C@@H]2C[C@@H](O)CN2C(=O)[C@@H](NC(=O)CN2CCN(c3ccc(Nc4cc(N(C)C(=O)Nc5c(Cl)cccc5Cl)ncn4)cc3)CC2)C(C)(C)C)cc1]

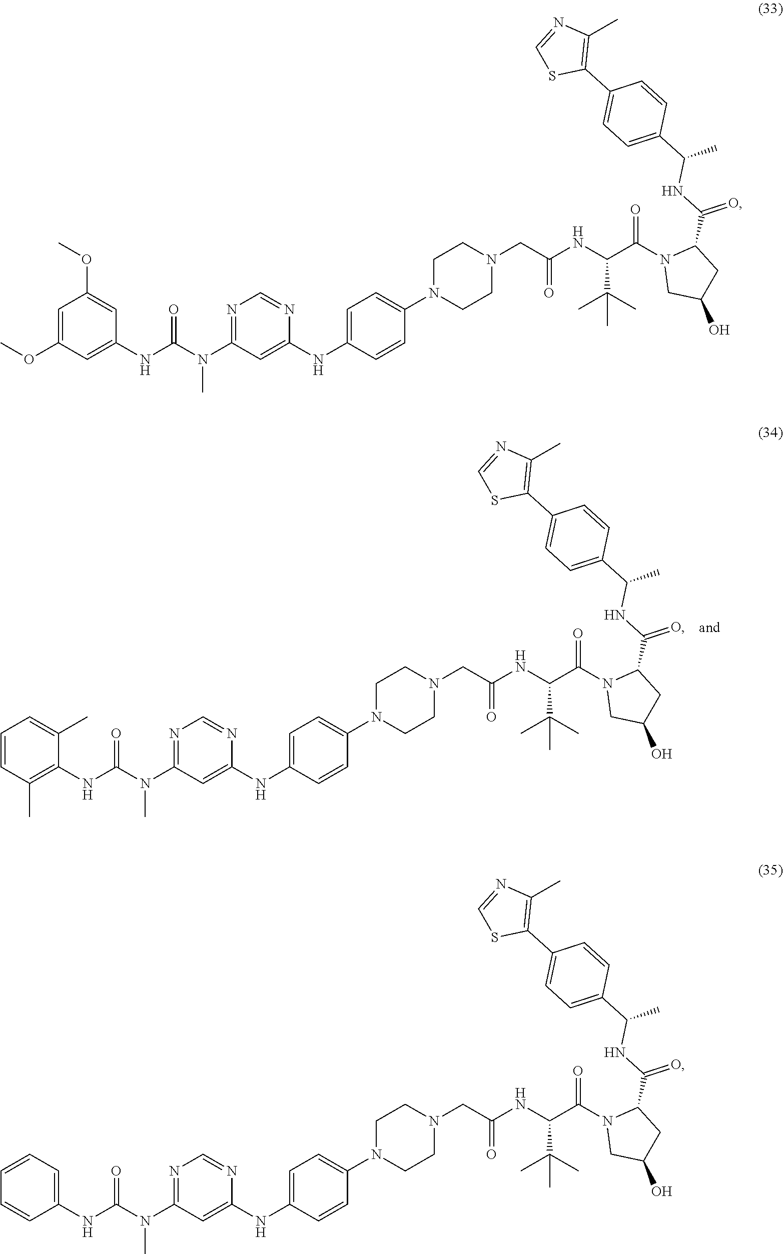 [SMILES: COc1cc(NC(=O)N(C)c2cc(Nc3ccc(N4CCN(CC(=O)N[C@H](C(=O)N5C[C@H](O)C[C@H]5C(=O)N[C@@H](C)c5ccc(-c6scnc6C)cc5)C(C)(C)C)CC4)cc3)ncn2)cc(OC)c1 ; Cc1cccc(C)c1NC(=O)N(C)c1cc(Nc2ccc(N3CCN(CC(=O)N[C@H](C(=O)N4C[C@H](O)C[C@H]4C(=O)N[C@@H](C)c4ccc(-c5scnc5C)cc4)C(C)(C)C)CC3)cc2)ncn1 ; Cc1ncsc1-c1ccc([C@H](C)NC(=O)[C@@H]2C[C@@H](O)CN2C(=O)[C@@H](NC(=O)CN2CCN(c3ccc(Nc4cc(N(C)C(=O)Nc5ccccc5)ncn4)cc3)CC2)C(C)(C)C)cc1]

or a pharmaceutically acceptable salt, or stereoisomer thereof.

Bispecific compounds of the present invention may be in the form of a free acid or free base, or a pharmaceutically acceptable salt. As used herein, the term “pharmaceutically acceptable” refers to a material, such as a carrier or diluent, which does not abrogate the biological activity or properties of the bispecific compound, and is relatively non-toxic, i.e., the material may be administered to a subject without causing undesirable biological effects (such as dizziness or gastric upset) or interacting in a deleterious manner with any of the components of the composition in which it is contained. The term “pharmaceutically acceptable salt” refers to a product obtained by reaction of the bispecific compound of the present invention with a suitable acid or a base. Examples of pharmaceutically acceptable salts of the bispecific compounds of this invention include those derived from suitable inorganic bases such as Li, Na, K, Ca, Mg, Fe. Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain bispecific compounds of the invention can form pharmaceutically acceptable salts with various organic bases such as lysine, arginine, guanidine, diethanolamine or metformin.

In some embodiments, the bispecific compound is an isotopic derivative in that it has at least one desired isotopic substitution of an atom, at an amount above the natural abundance of the isotope. i.e., enriched. In one embodiment, the bispecific compound includes deuterium or multiple deuterium atoms. Substitution with heavier isotopes such as deuterium, i.e. 2H, may afford certain therapeutic advantages resulting from greater metabolic stability, for example, increased in vivo half-life or reduced dosage requirements, and thus may be advantageous in some circumstances.

Bispecific compounds of the present invention may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R-) form is considered equivalent to administration of the compound in its (S-) form.

Accordingly, the bispecific compounds of the present invention may be made and used in the form of individual isomers and substantially free of other isomers, or in the form of a mixture of various isomers, e.g., racemic mixtures of stereoisomers.

### Methods of Synthesis

In another aspect, the present invention is directed to a method for making a bispecific compound of formula (I), or a pharmaceutically acceptable salt or stereoisomer thereof. Broadly, the inventive bispecific compounds or pharmaceutically-acceptable salts or stereoisomers thereof may be prepared by any process known to be applicable to the preparation of chemically related compounds. The bispecific compounds of the present invention will be better understood in connection with the synthetic schemes that described in various working examples and which illustrate non-limiting methods by which the bispecific compounds of the invention may be prepared.

### Pharmaceutical Compositions

Another aspect of the present invention is directed to a pharmaceutical composition that includes a therapeutically effective amount of a bispecific compound of formula (I) or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present invention to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may include one or more pharmaceutically acceptable excipients.

Broadly, bispecific compounds of formula (I) may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the bispecific compounds are formulated for oral or intravenous administration (e.g., systemic intravenous injection).

Accordingly, bispecific compounds of the present invention may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, bispecific compounds of the present invention may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, bispecific compounds of formula (I) may be administered in a local rather than systemic manner, for example, via injection of the conjugate directly into an organ, often in a depot preparation or sustained release formulation. In specific embodiments, long acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The bispecific compounds may be formulated for buccal or sublingual administration, examples of which include tablets, lozenges and gels.

The bispecific compounds may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Bispecific compounds of formula (I) may be formulated for topical administration which as used herein, refers to administration intradermally by application of the formulation to the epidermis. These types of compositions are typically in the form of ointments, pastes, creams, lotions, gels, solutions and sprays.

Representative examples of carriers useful in formulating compositions for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum comeum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of a bispecific compound of formula (I) or a pharmaceutically acceptable salt or a stereoisomer thereof; or a composition including a bispecific compound of formula (I) or a pharmaceutically acceptable salt or a stereoisomer thereof, effective in producing the desired therapeutic response in a particular patient suffering from a disease or disorder characterized or mediated by aberrant FGFR2 activity. The term “therapeutically effective amount” thus includes the amount of the compound of the invention or a pharmaceutically acceptable salt or a stereoisomer thereof, that when administered, induces a positive modification in the disease or disorder to be treated (e.g., to selectively inhibit/degrade FGFR2), or is sufficient to prevent development or progression of the disease or disorder, or alleviate to some extent, one or more of the symptoms of the disease or disorder being treated in a subject, or which simply kills or inhibits the growth of diseased (e.g., neuroblastoma) cells, or reduces the amount of FGFR2 in diseased cells.

The total daily dosage of the bispecific compounds and usage thereof may be decided in accordance with standard medical practice, e.g., by the attending physician using sound medical judgment. The specific therapeutically effective dose for any particular subject may depend upon a variety of factors including the disease or disorder being treated and the severity thereof (e.g., its present status); the age, body weight, general health, sex and diet of the subject; the time of administration, route of administration, and rate of excretion of the specific compound employed; the duration of the treatment; drugs used in combination or coincidental with the bispecific compound; and like factors well known in the medical arts (see, for example, Goodman and Gilman's. The Pharmacological Basis of Therapeutics, 10th Edition, A. Gilman, J. Hardman and L. Limbird, eds., McGraw-Hill Press, 155-173, 2001).

Bispecific compounds of formula (I) may be effective over a wide dosage range. In some embodiments, the total daily dosage (e.g., for adult humans) may range from about 0.001 to about 1600 mg, from 0.01 to about 1600 mg, from 0.01 to about 500 mg, from about 0.01 to about 100 mg, from about 0.5 to about 100 mg, from 1 to about 100-400 mg per day, from about 1 to about 50 mg per day, and from about 5 to about 40 mg per day, and in yet other embodiments from about 10 to about 30 mg per day. Individual dosages may be formulated to contain the desired dosage amount depending upon the number of times the compound is administered per day. By way of example, capsules may be formulated with from about 1 to about 200 mg of compound (e.g., 1, 2, 2.5, 3, 4, 5, 10, 15, 20, 25, 50, 100, 150, and 200 mg). In some embodiments, individual dosages may be formulated to contain the desired dosage amount depending upon the number of times the compound is administered per day.

### Methods of Use

In some aspects, the present invention is directed to methods of treating diseases or disorders involving aberrant (e.g., dysfunctional or dysregulated) FGFR2 activity, that entails administration of a therapeutically effective amount of a bispecific compound formula (I) or a pharmaceutically acceptable salt or stereoisomer thereof, to a subject in need thereof.

The diseases or disorders may be said to be characterized or mediated by aberrant (e.g., dysfunctional or dysregulated) FGFR2 activity (e.g., elevated levels of protein or otherwise functionally abnormal relative to a non-pathological state). A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the animal's state of health. In some embodiments, bispecific compounds of formula (I) may be useful in the treatment of cell proliferative diseases and disorders (e.g., cancer or benign neoplasms). As used herein, the term “cell proliferative disease or disorder” refers to the conditions characterized by deregulated or abnormal cell growth, or both, including noncancerous conditions such as neoplasms, precancerous conditions, benign tumors, and cancer.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present invention may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

Exemplary types of non-cancerous (e.g., cell proliferative) diseases or disorders that may be amenable to treatment with bispecific compounds of formula (I) include inflammatory diseases and conditions, autoimmune diseases, neurodegenerative diseases, heart diseases, viral diseases, chronic and acute kidney diseases or injuries, metabolic diseases, and allergic and genetic diseases.

Representative examples of specific non-cancerous diseases and disorders include rheumatoid arthritis, alopecia areata, lymphoproliferative conditions, autoimmune hematological disorders (e.g. hemolytic anemia, aplastic anemia, anhidrotic ectodermal dysplasia, pure red cell anemia and idiopathic thrombocytopenia), cholecystitis, acromegaly, rheumatoid spondylitis, osteoarthritis, gout, scleroderma, sepsis, septic shock, dacryoadenitis, cryopyrin associated periodic syndrome (CAPS), endotoxic shock, endometritis, gram-negative sepsis, keratoconjunctivitis sicca, toxic shock syndrome, asthma, adult respiratory distress syndrome, chronic obstructive pulmonary disease, chronic pulmonary inflammation, chronic graft rejection, hidradenitis suppurativa, inflammatory bowel disease, Crohn's disease, Behcet's syndrome, systemic lupus erythematosus, glomerulonephritis, multiple sclerosis, juvenile-onset diabetes, autoimmune uveoretinitis, autoimmune vasculitis, thyroiditis, Addison's disease, lichen planus, appendicitis, bullous pemphigoid, pemphigus vulgaris, pemphigus foliaceus, paraneoplastic pemphigus, myasthenia gravis, immunoglobulin A nephropathy, Hashimoto's disease, Sjogren's syndrome, vitiligo, Wegener granulomatosis, granulomatous orchitis, autoimmune oophoritis, sarcoidosis, rheumatic carditis, ankylosing spondylitis, Grave's disease, autoimmune thrombocytopenic purpura, psoriasis, psoriatic arthritis, eczema, dermatitis herpetiformis, ulcerative colitis, pancreatic fibrosis, hepatitis, hepatic fibrosis, CD14 mediated sepsis, non-CD14 mediated sepsis, acute and chronic renal disease, irritable bowel syndrome, pyresis, restenosis, cervicitis, stroke and ischemic injury, neural trauma, acute and chronic pain, allergic rhinitis, allergic conjunctivitis, chronic heart failure, congestive heart failure, acute coronary syndrome, cachexia, malaria, leprosy, leishmaniosis, Lyme disease, Reiter's syndrome, acute synovitis, muscle degeneration, bursitis, tendonitis, tenosynovitis, herniated, ruptured, or prolapsed intervertebral disk syndrome, osteopetrosis, rhinosinusitis, thrombosis, silicosis, pulmonary sarcosis, bone resorption diseases, such as osteoporosis, fibromyalgia, AIDS and other viral diseases such as Herpes Zoster, Herpes Simplex I or II, influenza virus and cytomegalovirus, diabetes Type I and II, obesity, insulin resistance and diabetic retinopathy, 22q11.2 deletion syndrome, Angelman syndrome, Canavan disease, celiac disease, Charcot-Marie-Tooth disease, color blindness, Cri du chat, Down syndrome, cystic fibrosis, Duchenne muscular dystrophy, haemophilia, Klinefleter's syndrome, neurofibromatosis, phenylketonuria, Prader-Willi syndrome, sickle cell disease, Tay-Sachs disease, Turner syndrome, urea cycle disorders, thalassemia, otitis, pancreatitis, parotitis, pericarditis, peritonitis, pharyngitis, pleuritis, phlebitis, pneumonitis, uveitis, polymyositis, proctitis, interstitial lung fibrosis, dermatomyositis, atherosclerosis, arteriosclerosis, amyotrophic lateral sclerosis, asociality, varicosis, vaginitis, depression, and Sudden Infant Death Syndrome.

In some embodiments, the bispecific compounds may be useful in the treatment of non-cancerous neurodegenerative diseases and disorders. As used herein, the term “neurodegenerative diseases and disorders” refers to the conditions characterized by progressive degeneration or death of nerve cells, or both, including problems with movement (ataxias), or mental functioning (dementias). Representative examples of such diseases and disorders include Alzheimer's disease (AD) and AD-related dementias, Parkinson's disease (PD) and PD-related dementias, prion disease, motor neuron diseases (MND), Huntington's disease (HD), Pick's syndrome, spinocerebellar ataxia (SCA), spinal muscular atrophy (SMA), primary progressive aphasia (PPA), amyotrophic lateral sclerosis (ALS), traumatic brain injury (TBI), multiple sclerosis (MS), dementias (e.g., vascular dementia (VaD), Lewy body dementia (LBD), semantic dementia, and frontotemporal lobar dementia (FTD).

In other embodiments, the methods are directed to treating subjects having cancer. Broadly, the bispecific compounds of the present invention may be effective in the treatment of carcinomas (solid tumors including both primary and metastatic tumors), sarcomas, melanomas, and hematological cancers (cancers affecting blood including lymphocytes, bone marrow and/or lymph nodes) such as leukemia, lymphoma and multiple myeloma. Adult tumors/cancers and pediatric tumors/cancers are included. The cancers may be vascularized, or not yet substantially vascularized, or non-vascularized tumors.

Representative examples of cancers includes adenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer, urinary bladder cancer, brain cancer (e.g., gliomas and glioblastomas such as brain stem glioma, gestational trophoblastic tumor glioma, cerebellar astrocytoma, cerebral astrocytoma/malignant glioma, ependymoma, medulloblastoma, supratentorial primitive neuroectodeimal tumors, visual pathway and hypothalamic glioma), breast cancer, bronchial adenomas/carcinoids, carcinoid tumor, nervous system cancer (e.g., central nervous system cancer, central nervous system lymphoma), cervical cancer, chronic myeloproliferative disorders, colorectal cancer (e.g., colon cancer, rectal cancer), lymphoid neoplasm, mycosis fungoids, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastrointestinal cancer (e.g., stomach cancer, small intestine cancer, gastrointestinal carcinoid tumor, gastrointestinal stromal tumor (GIST)), cholangiocarcinoma, germ cell tumor, ovarian germ cell tumor, head and neck cancer, neuroendocrine tumors, Hodgkin's lymphoma, Ann Arbor stage III and stage IV childhood Non-Hodgkin's lymphoma, ROSi-positive refractory Non-Hodgkin's lymphoma, leukemia, lymphoma, multiple myeloma, hypopharyngeal cancer, intraocular melanoma, ocular cancer, islet cell tumors (endocrine pancreas), renal cancer (e.g., Wilm's Tumor, renal cell carcinoma), liver cancer, lung cancer (e.g., non-small cell lung cancer and small cell lung cancer), ALK-positive anaplastic large cell lymphoma, ALK-positive advanced malignant solid neoplasm, Waldenstrom's macroglobulinemia, melanoma, intraocular (eye) melanoma, merkel cell carcinoma, mesothelioma, metastatic squamous neck cancer with occult primary, multiple endocrine neoplasia (MEN), myelodysplastic syndromes, myelodysplastic/myeloproliferative diseases, nasopharyngeal cancer, neuroblastoma, oral cancer (e.g., mouth cancer, lip cancer, oral cavity cancer, tongue cancer, oropharyngeal cancer, throat cancer, laryngeal cancer), ovarian cancer (e.g., ovarian epithelial cancer, ovarian germ cell tumor, ovarian low malignant potential tumor), pancreatic cancer, islet cell pancreatic cancer, paranasal sinus and nasal cavity cancer, parathyroid cancer, penile cancer, pharyngeal cancer, pheochromocytoma, pineoblastoma, metastatic anaplastic thyroid cancer, undifferentiated thyroid cancer, papillary thyroid cancer, pituitary tumor, plasma cell neoplasm/multiple myeloma, pleuropulmonary blastoma, prostate cancer, retinoblastoma, rhabdomyosarcoma, salivary gland cancer, uterine cancer (e.g., endometrial uterine cancer, uterine sarcoma, uterine corpus cancer), squamous cell carcinoma, testicular cancer, thymoma, thymic carcinoma, thyroid cancer, juvenile xanthogranuloma, transitional cell cancer of the renal pelvis and ureter and other urinary organs, urethral cancer, gestational trophoblastic tumor, vaginal cancer, vulvar cancer, hepatoblastoma, rhabdoid tumor, and Wilms tumor.

Sarcomas that may be treatable with compounds of the present invention include both soft tissue and bone cancers alike, representative examples of which include osteosarcoma or osteogenic sarcoma (bone) (e.g., Ewing's sarcoma), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue), mesenchymous or mixed mesodermal tumor (mixed connective tissue types), and histiocytic sarcoma (immune cancer).

In some embodiments, methods of the present invention entail treatment of subjects having cell proliferative diseases or disorders of the hematological system, liver, brain, lung, colon, pancreas, prostate, ovary, breast, skin, and endometrium.

As used herein, “cell proliferative diseases or disorders of the hematological system” include lymphoma, leukemia, myeloid neoplasms, mast cell neoplasms, myelodysplasia, benign monoclonal gammopathy, polycythemia vera, chronic myelocytic leukemia, agnogenic myeloid metaplasia, and essential thrombocythemia. Representative examples of hematologic cancers may thus include multiple myeloma, lymphoma (including T-cell lymphoma, Hodgkin's lymphoma, non-Hodgkin's lymphoma (diffuse large B-cell lymphoma (DLBCL), follicular lymphoma (FL), mantle cell lymphoma (MCL) and ALK+ anaplastic large cell lymphoma (e.g., B-cell non-Hodgkin's lymphoma selected from diffuse large B-cell lymphoma (e.g., germinal center B-cell-like diffuse large B-cell lymphoma or activated B-cell-like diffuse large B-cell lymphoma), Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, metastatic pancreatic adenocarcinoma, refractory B-cell non-Hodgkin's lymphoma, and relapsed B-cell non-Hodgkin's lymphoma, childhood lymphomas, and lymphomas of lymphocytic and cutaneous origin, e.g., small lymphocytic lymphoma, leukemia, including childhood leukemia, hairy-cell leukemia, acute lymphocytic leukemia, acute myelocytic leukemia, acute myeloid leukemia (e.g., acute monocytic leukemia), chronic lymphocytic leukemia, small lymphocytic leukemia, chronic myelocytic leukemia, chronic myelogenous leukemia, and mast cell leukemia, myeloid neoplasms and mast cell neoplasms.

As used herein, “cell proliferative diseases or disorders of the liver” include all forms of cell proliferative disorders affecting the liver. Cell proliferative disorders of the liver may include liver cancer (e.g., hepatocellular carcinoma, intrahepatic cholangiocarcinoma and hepatoblastoma), a precancer or precancerous condition of the liver, benign growths or lesions of the liver, and malignant growths or lesions of the liver, and metastatic lesions in tissue and organs in the body other than the liver. Cell proliferative disorders of the liver may include hyperplasia, metaplasia, and dysplasia of the liver.

As used herein, “cell proliferative diseases or disorders of the brain” include all forms of cell proliferative disorders affecting the brain. Cell proliferative disorders of the brain may include brain cancer (e.g., gliomas, glioblastomas, meningiomas, pituitary adenomas, vestibular schwannomas, and primitive neuroectodermal tumors (medulloblastomas)), a precancer or precancerous condition of the brain, benign growths or lesions of the brain, and malignant growths or lesions of the brain, and metastatic lesions in tissue and organs in the body other than the brain. Cell proliferative disorders of the brain may include hyperplasia, metaplasia, and dysplasia of the brain.

As used herein, “cell proliferative diseases or disorders of the lung” include all forms of cell proliferative disorders affecting lung cells. Cell proliferative disorders of the lung include lung cancer, precancer and precancerous conditions of the lung, benign growths or lesions of the lung, hyperplasia, metaplasia, and dysplasia of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), squamous cell carcinoma, adenocarcinoma, small cell carcinoma, large cell carcinoma, squamous cell carcinoma, and mesothelioma. Lung cancer can include “scar carcinoma”, bronchioveolar carcinoma, giant cell carcinoma, spindle cell carcinoma, and large cell neuroendocrine carcinoma. Lung cancer also includes lung neoplasms having histologic and ultrastructural heterogeneity (e.g., mixed cell types). In some embodiments, bispecific compounds of the present invention may be used to treat non-metastatic or metastatic lung cancer (e.g., NSCLC, ALK-positive NSCLC, NSCLC harboring ROS1 Rearrangement, Lung Adenocarcinoma, and Squamous Cell Lung Carcinoma).

As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer, malignant colon neoplasms, carcinoma in situ, typical carcinoid tumors, and atypical carcinoid tumors, adenocarcinoma, squamous cell carcinoma, and squamous cell carcinoma. Colon cancer can be associated with a hereditary syndrome such as hereditary nonpolyposis colorectal cancer, familiar adenomatous polyposis, MYH associated polyposis, Gardner's syndrome, Peutz-Jeghers syndrome, Turcot's syndrome and juvenile polyposis. Cell proliferative disorders of the colon may also be characterized by hyperplasia, metaplasia, or dysplasia of the colon.

As used herein, “cell proliferative diseases or disorders of the pancreas” include all forms of cell proliferative disorders affecting pancreatic cells. Cell proliferative disorders of the pancreas may include pancreatic cancer, a precancer or precancerous condition of the pancreas, hyperplasia of the pancreas, dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell types).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the ovary” include all forms of cell proliferative disorders affecting cells of the ovary. Cell proliferative disorders of the ovary may include a precancer or precancerous condition of the ovary, benign growths or lesions of the ovary, ovarian cancer, and metastatic lesions in tissue and organs in the body other than the ovary. Cell proliferative disorders of the ovary may include hyperplasia, metaplasia, and dysplasia of the ovary.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast. Cell proliferative disorders of the breast may include hyperplasia, metaplasia, and dysplasia of the breast.

As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the skin.

As used herein, “cell proliferative diseases or disorders of the endometrium” include all forms of cell proliferative disorders affecting cells of the endometrium. Cell proliferative disorders of the endometrium may include a precancer or precancerous condition of the endometrium, benign growths or lesions of the endometrium, endometrial cancer, and metastatic lesions in tissue and organs in the body other than the endometrium. Cell proliferative disorders of the endometrium may include hyperplasia, metaplasia, and dysplasia of the endometrium

In some embodiments, the disease or disorder is liver cancer. In other embodiments, the disease or disorder is biliary tract cancer (BTC). In other embodiments, the disease or disorder is intrahepatic cholangiocarcinoma (ICC) or extrahepatic cholangiocarcinoma (ECC).

The bispecific compounds of formula (I) may be administered to a patient, e.g., a cancer patient, as a monotherapy or by way of combination therapy, and as a front-line therapy or a follow-on therapy for patients who are unresponsive to front line therapy. Therapy may be “first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments. Therapy may also be given to patients who have had previous treatments which have been partially successful but are intolerant to the particular treatment. Therapy may also be given as an adjuvant treatment, i.e., to prevent reoccurrence of cancer in patients with no currently detectable disease or after surgical removal of a tumor. Thus, in some embodiments, the compound may be administered to a patient who has received another therapy, such as chemotherapy, radioimmunotherapy, surgical therapy, immunotherapy, radiation therapy, targeted therapy or any combination thereof.

The methods of the present invention may entail administration of bispecific compounds of formula (I) or pharmaceutical compositions thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails a 28-day cycle which includes daily administration for 3 weeks (21 days). In other embodiments, the bispecific compound may be dosed twice a day (BID) over the course of two and a half days (for a total of 5 doses) or once a day (QD) over the course of two days (for a total of 2 doses). In other embodiments, the bispecific compound may be dosed once a day (QD) over the course of five days.

### Combination Therapy

Bispecific compounds of formula (I) may be used in combination or concurrently with at least one other active agent, e.g., anti-cancer agent or regimen, in treating diseases and disorders. The terms “in combination” and “concurrently in this context mean that the agents are co-administered, which includes substantially contemporaneous administration, by way of the same or separate dosage forms, and by the same or different modes of administration, or sequentially, e.g., as part of the same treatment regimen, or by way of successive treatment regimens. Thus, if given sequentially, at the onset of administration of the second compound, the first of the two compounds is in some cases still detectable at effective concentrations at the site of treatment. The sequence and time interval may be determined such that they can act together (e.g., synergistically to provide an increased benefit than if they were administered otherwise). For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

In some embodiments, the treatment regimen may include administration of a bispecific compound of formula (I) or a pharmaceutically acceptable salt or stereoisomer in combination with one or more additional therapeutics known for use in treating the disease or disorder (e.g., cancer). The dosage of the additional anticancer therapeutic may be the same or even lower than known or recommended doses. See, Hardman et al., eds., Goodman & Gilman's The Pharmacological Basis Of Basis Of Therapeutics, 10th ed., McGraw-Hill, New York, 2001; Physician's Desk Reference 60th ed., 2006. For example, anti-cancer agents that may be used in combination with the bispecific compounds are known in the art. See, e.g., U.S. Pat. No. 9,101,622 (Section 5.2 thereof) and U.S. Pat. No. 9,345,705 B2 (Columns 12-18 thereof). Representative examples of additional active agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immunomodulators, therapeutic antibodies (e.g., mono-specific and bispecific antibodies) and CAR-T therapy.

In some embodiments, the bispecific compound of formula (I) may be used in combination with other anti-cancer agents, examples of which include Paclitaxel (e.g., ovarian cancer, breast cancer, lung cancer, Kaposi sarcoma, cervical cancer, and pancreatic cancer), Topotecan (e.g., ovarian cancer and lung cancer), Irinotecan (e.g., colon cancer, and small cell lung cancer), Etoposide (e.g., testicular cancer, lung cancer, lymphomas, and nonlymphocytic leukemia), Vincristine (e.g., leukemia), Leucovorin (e.g., colon cancer), Altretamine (e.g., ovarian cancer), Daunorubicin (e.g., acute myeloid leukemia (AML), acute lymphocytic leukemia (ALL), chronic myelogenous leukemia (CML), and Kaposi's sarcoma), Trastuzumab (e.g., breast cancer, stomach cancer, and esophageal cancer), Rituximab (e.g., non-Hodgkin's lymphoma), Cetuximab (e.g., colorectal cancer, metastatic non-small cell lung cancer and head and neck cancer), Pertuzumab (e.g., metastatic HER2-positive breast cancer), Alemtuzumab (e.g., chronic lymphocytic leukemia (CLL), cutaneous T-cell lymphoma (CTCL) and T-cell lymphoma), Panitumumab (e.g., colon and rectum cancer), Tamoxifen (e.g., breast cancer), Fulvestrant (e.g., breast cancer), Letrazole (e.g., breast cancer), Exemestane (e.g., breast cancer), Azacytidine (e.g., myelodysplastic syndromes), Mitomycin C (e.g., gastro-intestinal cancers, anal cancers, and breast cancers), Dactinomycin (e.g., Wilms tumor, rhabdomyosarcoma, Ewing's sarcoma, trophoblastic neoplasm, testicular cancer, and ovarian cancer), Erlotinib (e.g., non-small cell lung cancer and pancreatic cancer), Sorafenib (e.g., kidney cancer and liver cancer), Temsirolimus (e.g., kidney cancer), Bortezomib (e.g., multiple myeloma and mantle cell lymphoma), Pegaspargase (e.g., acute lymphoblastic leukemia), Cabometyx (e.g., hepatocellular carcinoma, medullary thyroid cancer, and renal cell carcinoma), Keytruda (e.g., cervical cancer, gastric cancer, hepatocellular carcinoma, Hodgkin lymphoma, melanoma, Merkel cell carcinoma, non-small cell lung cancer, urothelial carcinoma, and squamous cell carcinoma of the head and neck), Nivolumab (e.g., colorectal cancer, hepatocellular carcinoma, melanoma, non-small cell lung cancer, renal cell carcinoma, small cell lung cancer, and urothelial carcinoma), and Regorafenib (e.g., colorectal cancer, gastrointestinal stromal tumor, and hepatocellular carcinoma).

In some embodiments, the bispecific compound of formula (I) and the additional (e.g., anticancer) therapeutic may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The two or more (e.g., anticancer) therapeutics may be administered within the same patient visit.

When the active components of the combination are not administered in the same pharmaceutical composition, it is understood that they can be administered in any order to a subject in need thereof. For example, a compound of the present invention can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours. 96 hours, 1 week, 2 weeks, 3 weeks, 4 weeks, 5 weeks, 6 weeks, 8 weeks, or 12 weeks after) the administration of the additional anticancer therapeutic, to a subject in need thereof. In various aspects, the anticancer therapeutics are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one example, the (e.g., anticancer) therapeutics are administered within the same office visit. In another example, the combination anticancer therapeutics may be administered at 1 minute to 24 hours apart.

In some embodiments involving cancer treatment, the bispecific compound of formula (I) and the additional anti-cancer agent or therapeutic are cyclically administered. Cycling therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

### Pharmaceutical Kits

The present bispecific compounds and/or compositions containing them may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain the bispecific compound of formula (I) or a pharmaceutical composition thereof. The kits or pharmaceutical systems of the invention may also include printed instructions for using the compounds and compositions.

These and other aspects of the present invention will be further appreciated upon consideration of the following Examples, which are intended to illustrate certain particular embodiments of the invention but are not intended to limit its scope, as defined by the claims.

### Examples

### Example 1: Synthesis of Thalidomide-Based Intermediates

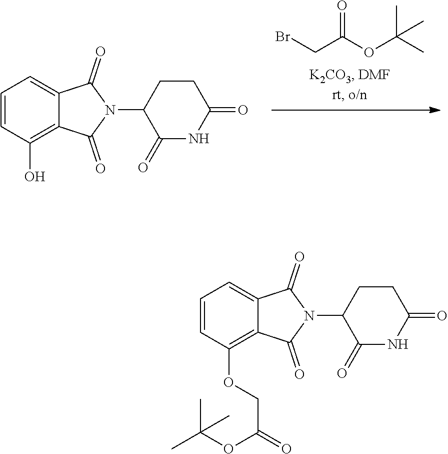

### tert-Butyl 2-((2-(2,6-dioxopiperidin-3-yl)-1,3-dioxoisoindolin-4-yl)oxy)acetate

t-Butyl bromoacetate (199 mg, 1.02 mmol) was added to a mixture of 2-(2,6-dioxopiperidin-3-yl)-4-hydroxyisoindoline-1,3-dione (200 mg, 0.73 mmol) and K₂CO₃ (304 mg, 2.20 mmol) in 3 mL DMF at room temperature. The mixture was stirred overnight and then quenched with water. The aqueous mixture was then extracted with 3×5 mL ethyl acetate, washed with brine, dried using Na₂SO₄, and concentrated. Purification by silica gel chromatography provided tert-butyl 2-((2-(2,6-dioxopiperidin-3-yl)-1,3-dioxoisoindolin-4-yl)oxy)acetate (245 mg, 0.63 mmol, 86%) as a white crystalline solid. LC/MS m/z calculated for [M+2H-tBu]⁺333.1, found 333.1.

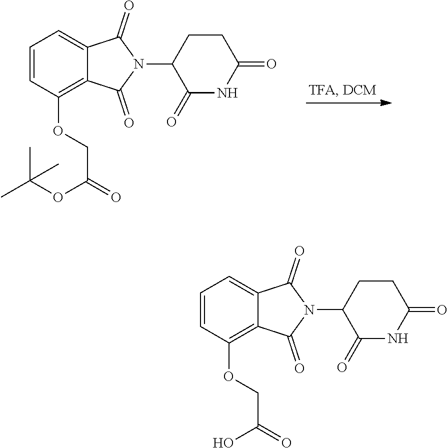 [SMILES: CC(C)(C)OC(=O)COc1cccc2c1C(=O)N(C1CCC(=O)NC1=O)C2=O ; O=C(O)COc1cccc2c1C(=O)N(C1CCC(=O)NC1=O)C2=O]

### 2-((2-(2,6-Dioxopiperidin-3-yl)-1,3-dioxoisoindolin-4-yl)oxy)acetic acid

tert-Butyl 2-((2-(2,6-dioxopiperidin-3-yl)-1,3-dioxoisoindolin-4-yl)oxy)acetate (140 mg, 0.036 mmol) was dissolved in 1 mL DCM and 1 mL TFA, and stirred for 1 hour at room temperature. The solvent was evaporated to obtain 2-((2-(2,6-dioxopiperidin-3-yl)-1,3-dioxoisoindolin-4-yl)oxy)acetic acid (121 mg, 0.036 mmol, 101%) as a white solid. LC/MS m/z calculated for [M+H]⁺ 333.1, found 332.7.

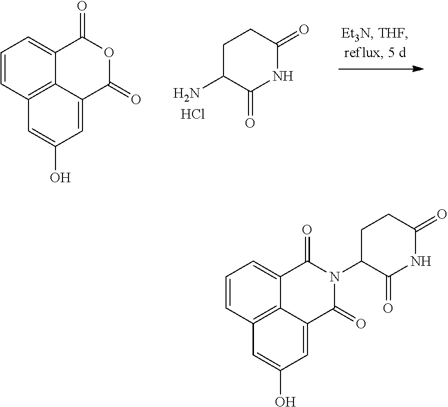 [SMILES: O=C1OC(=O)c2cc(O)cc3cccc1c23 ; Cl ; NC1CCC(=O)NC1=O ; O=C1CCC(N2C(=O)c3cccc4cc(O)cc(c34)C2=O)C(=O)N1]

### 2-(2,6-Dioxopiperidin-3-yl)-5-hydroxy-1H-benzo[de]isoquinoline-1,3(2H)-dione

3-Hydroxy-1,8-naphthalic anhydride (2.14 g, 10.0 mmol) and 3-aminopiperidine-2,6-dione (1.65 g, 10.0 mmol) were dissolved in THF (40 mL) at room temperature, and triethylamine (2.78 mL. 20.0 mmol) was added. The suspension was then refluxed for 5 days, with a green precipitate forming in the first 24 hours and eventually turning black. The solvent was evaporated, water was added, and the mixture was acidified, and stirred for 1 hour. The suspension was then filtered to provide the title compound (3.44 g, 9.53 mmol, 95%) as a green solid. LC/MS m/z calculated for [M+H]⁺ 325.1, found 325.1.

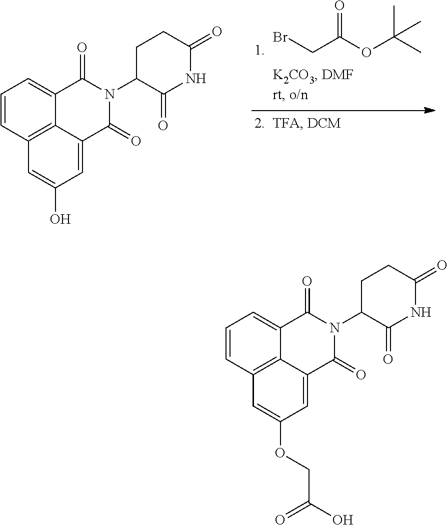

### tert-Butyl 2-((2-(2,6-dioxopiperidin-3-yl)-1,3-dioxo-2,3-dihydro-1H-benzo[de]isoquinolin-5-yl)oxy)acetate

2-(2,6-Dioxopiperidin-3-yl)-5-hydroxy-1H-benzo[de]isoquinoline-1,3(2H)-dione (361 mg, 1.1 mmol) was suspended in 3 mL DMF, followed by addition of K₂CO₃ (276 mg, 2.0 mmol) and t-butyl bromoacetate (234 mg, 1.2 mmol). The blue suspension was stirred at room temperature for 4 hours, at which point an additional 1.0 mmol of t-butyl bromoacetate was added. After continuing to stir overnight, water was added, and the suspension was filtered to provide the tert-butyl ester (464 mg, 1.06 mmol, 95%) as a light gray solid. LC/MS m/z calculated for [M+2H-tBu]+383.1, found 383.2.

This ester was then dissolved in DCM (1 mL) and TFA (1 mL) was added, and the solution stirred for 2 hours at room temperature. The solvent was then removed and the product dried to yield the title compound without further purification. LC/MS m/z calculated [M+H]⁺ 383.08, found 383.19.

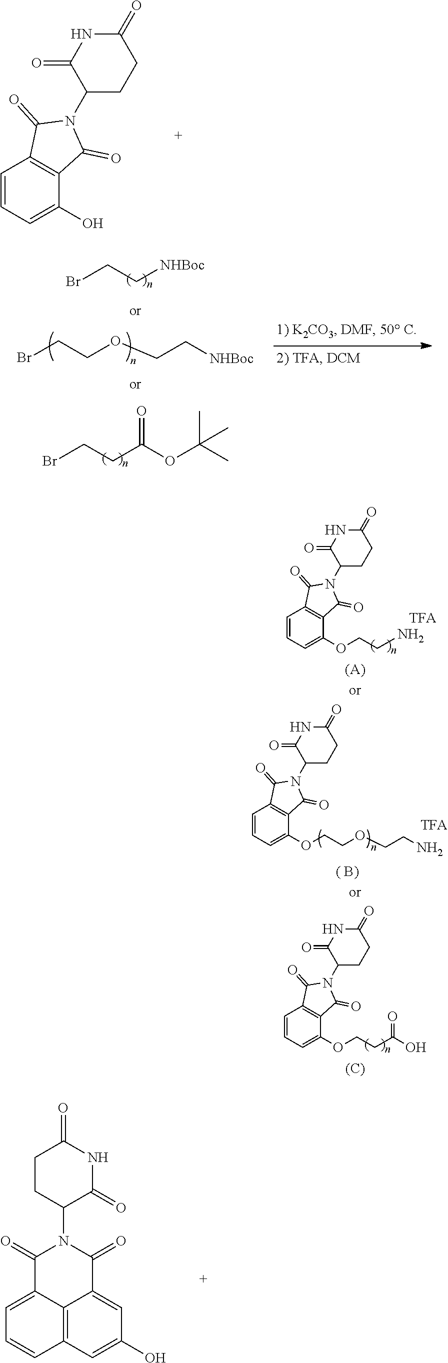

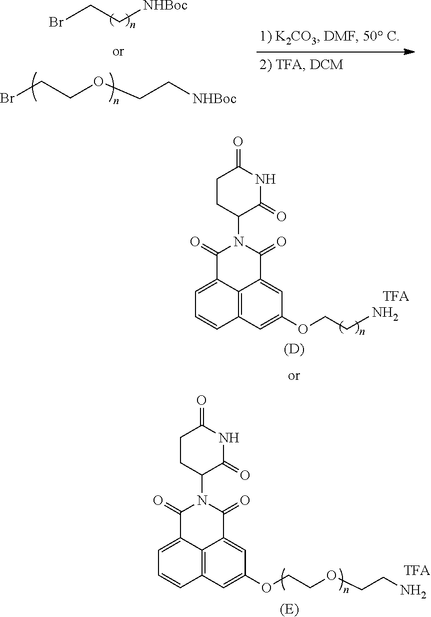

### Thalidomide-Based Intermediates (A-E)

2-(2,6-Dioxopiperidin-3-yl)-4-hydroxyisoindoline-1,3-dione or 2-(2,6-dioxopiperidin-3-yl)-4-hydroxyisoindoline-1,3-dione (1 eq.) was dissolved in DMF, treated with K₂CO₃ (2 eq.), the appropriate alkyl bromide linker (1.0 eq.) was added, and the mixture stirred overnight at 50° C. The reaction was quenched with water and extracted with EtOAc. Combined extracts were washed with brine, dried with Na₂SO₄, then concentrated and purified by silica gel chromatography to obtain the protected amine or ester. These intermediates were then dissolved in 1:1 DCM:TFA and stirred for 2 hours at room temperature before being concentrated and dried to provide compounds of type A-E.

### Example 2: Synthesis of 2-(4-{4-[(6-{[(2,6-Dichloro-3,5-dimethoxyphenyl)carbamoyl](methyl)amino}pyrimidin-4-yl)amino]phenyl}piperazin-1-yl)-N-{2-[2-(2-{[3-(2,6-dioxopiperidin-3-yl)-2,4-dioxo-3-azatricyclo[7.3.1.0^{5,13}]trideca-1(12),5,7,9(13),10-pentaen-7-yl]oxy}ethoxy)ethoxy]ethyl}acetamide (1)

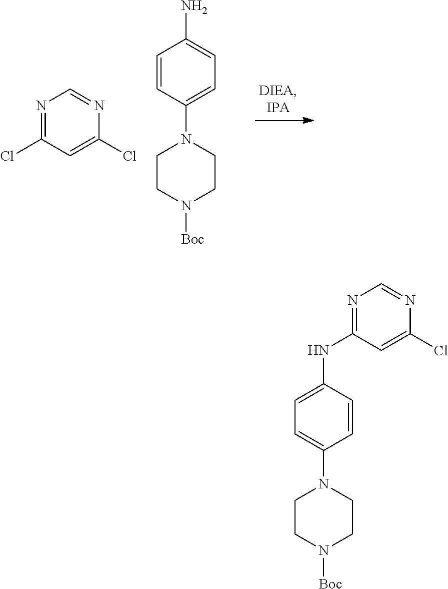

### tert-Butyl 4-(4-((6-chloropyrimidin-4-yl)amino)phenyl)piperazine-1-carboxylate

tert-Butyl 4-(4-aminophenyl)piperazine-1-carboxylate (2.0 g, 7.21 mmol) was added to 4,6-dichloropyrimidine (1.61 g, 10.8 mmol) in DIEA (1.88 mL, 10.8 mmol) and isopropanol (15 mL). The purple solution was then stirred at room temperature overnight. The solvent was evaporated and the residue purified by silica gel chromatography to provide the title compound as a maroon solid (2.75 g, 7.05 mmol, 98%). LC/MS m/z calculated [M+H]⁺ 390.16, found 390.30.

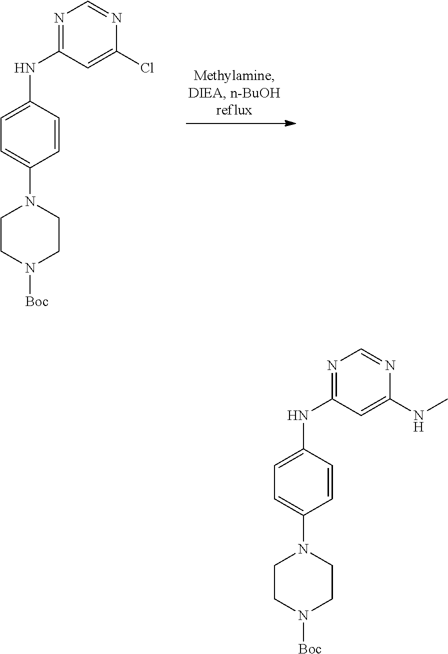 [SMILES: CC(C)(C)OC(=O)N1CCN(c2ccc(Nc3cc(Cl)ncn3)cc2)CC1 ; CNc1cc(Nc2ccc(N3CCN(C(=O)OC(C)(C)C)CC3)cc2)ncn1]

### tert-Butyl 4-(4-((6-(methylamino)pyrimidin-4-yl)amino)phenyl)piperazine-1-carboxylate

tert-Butyl 4-(4-((6-chloropyrimidin-4-yl)amino)phenyl)piperazine-1-carboxylate (1.0 g, 2.6 mmol) was suspended in 1-butanol (20 mL) and DIEA (910 uL, 5.2 mmol) was added, followed by methylamine (1.28 mmol, 2M in THF). The reaction vessel was then sealed, and the mixture heated at 120° C. overnight. The solvent was evaporated to yield the title compound without further purification. LC/MS m/z calculated [M+H]⁺ 385.23, found 385.07.

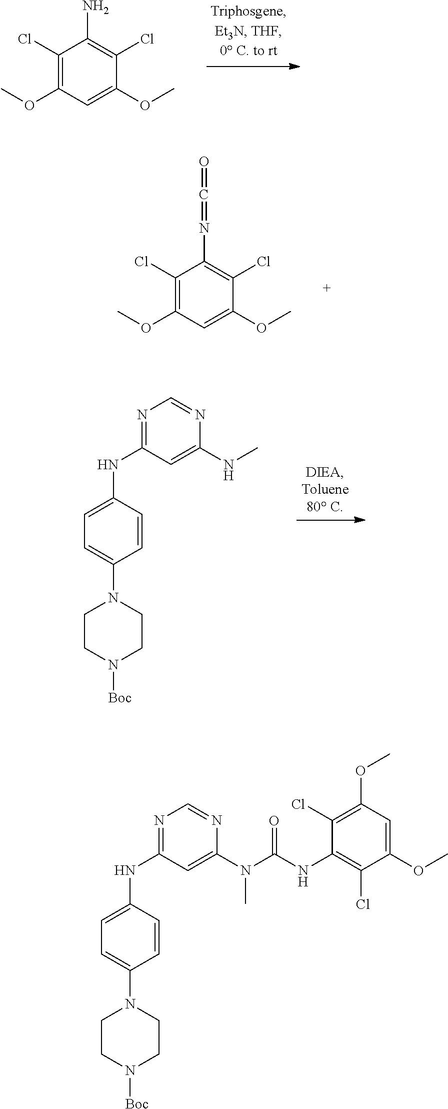

### tert-Butyl 4-(4-((6-(3-(2,6-dichloro-3,5-dimethoxyphenyl)-1,3-dimethylureido)pyrimidin-4-yl)amino)phenyl)piperazine-1-carboxylate

2,6-Dichloro-3,5-dimethoxyaniline (444 mg, 2.0 mmol) and triphosgene (237 mg, 0.80 mmol) were dissolved in THF, At 0° C., DIEA was slowly added, and a white precipitate formed as the reaction was slowly allowed to warm to room temperature. After 1 hour, the solvent was evaporated, and the crude isocyanate resuspended in toluene (10 mL). DIEA (1.39 mL, 8.0 mmol) and tert-butyl 4-(4-((6-(methylamino)pyrimidin-4-yl)amino)phenyl)piperazine-1-carboxylate (768 mg, 2.0 mmol) were added, and the mixture stirred at 80° C. overnight. The solvent was evaporated, and the crude product purified by silica gel chromatography to provide the title compound (828 mg, 1.31 mmol, 66%). LC/MS m/z calculated [M+H]⁺ 631.22, found 631.90.

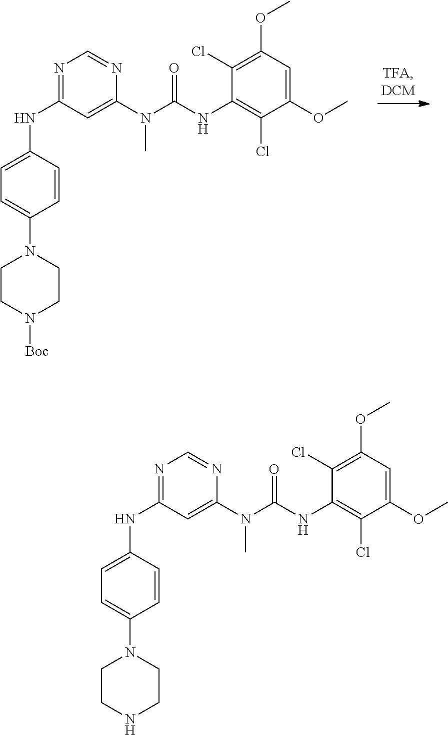 [SMILES: COc1cc(OC)c(Cl)c(NC(=O)N(C)c2cc(Nc3ccc(N4CCN(C(=O)OC(C)(C)C)CC4)cc3)ncn2)c1Cl ; COc1cc(OC)c(Cl)c(NC(=O)N(C)c2cc(Nc3ccc(N4CCNCC4)cc3)ncn2)c1Cl]

### 1-(2,6-Dichloro-3,5-dimethoxyphenyl)-1,3-dimethyl-3-(6-((4-(piperazin-1-yl)phenyl)amino)pyrimidin-4-yl)urea

tert-Butyl 4-(4-((6-(3-(2,6-dichloro-3,5-dimethoxyphenyl)-1,3-dimethylureido)pyrimidin-4-yl)amino)phenyl)piperazine-1-carboxylate (828 mg, 1.31 mmol) was dissolved in DCM (1 mL) and TFA (1 mL) was added. The solution was stirred for 2 hours and the solvent was evaporated. The residue was then dissolved in THF and stirred with sat. aqueous NaHCO₃ for 30 minutes. The brown precipitate was then filtered, washed with water and dried to provide the title compound as a yellow solid (611 mg, 1.07 mmol). LC/MS m/z calculated [M+H]⁺ 531.17, found 531.78.

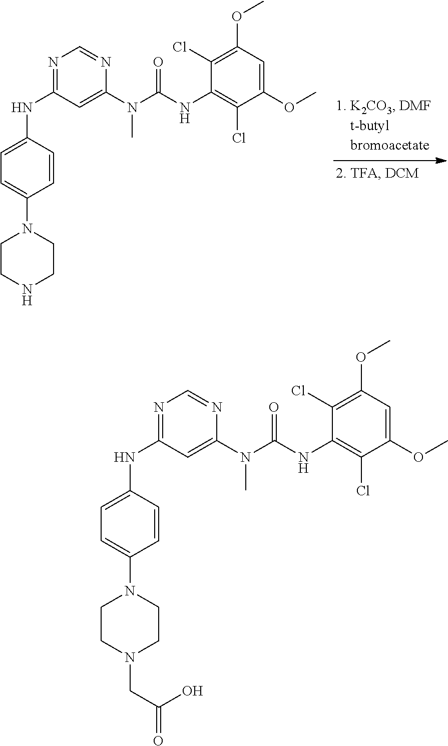 [SMILES: COc1cc(OC)c(Cl)c(NC(=O)N(C)c2cc(Nc3ccc(N4CCNCC4)cc3)ncn2)c1Cl ; COc1cc(OC)c(Cl)c(NC(=O)N(C)c2cc(Nc3ccc(N4CCN(CC(=O)O)CC4)cc3)ncn2)c1Cl]

### 2-(4-(4-((6-(3-(2,6-Dichloro-3,5-dimethoxyphenyl)-1,3-dimethylureido)pyrimidin-4-yl)amino)phenyl)piperazin-1-yl)acetic acid

1-(2,6-Dichloro-3,5-dimethoxyphenyl)-1,3-dimethyl-3-(6-((4-(piperazin-1-yl)phenyl)amino)pyrimidin-4-yl)urea (42 mg, 0.079) was dissolved in DMF (1 mL) and treated with K₂CO₃ (44 mg, 0.32 mmol). t-butyl bromoacetate was added (15 mg, 0.079 mmol), the mixture was stirred at room temperature overnight, then water was added, followed by extraction with ethyl acetate. Combined extracts were washed with brine, dried with Na₂SO₄ and purified by silica gel chromatography to provide the t-butyl ester. LC/MS m/z calculated [M+H]⁺ 646,22, found 646.30.

tert-Butyl 2-(4-(4-((6-(3-(2,6-dichloro-3,5-dimethoxyphenyl)-1,3-dimethylureido)pyrimidin-4-yl)amino)phenyl)piperazin-1-yl)acetate was then dissolved in DCM (1 mL) and TFA (1 mL) was added. The solution was stirred for 2 hours and the solvent was evaporated to provide the title compound (34.4 mg, 0.058 mmol, 74% over 2 steps). LC/MS m/z calculated [M+H]⁺ 590.16, found 590.39.

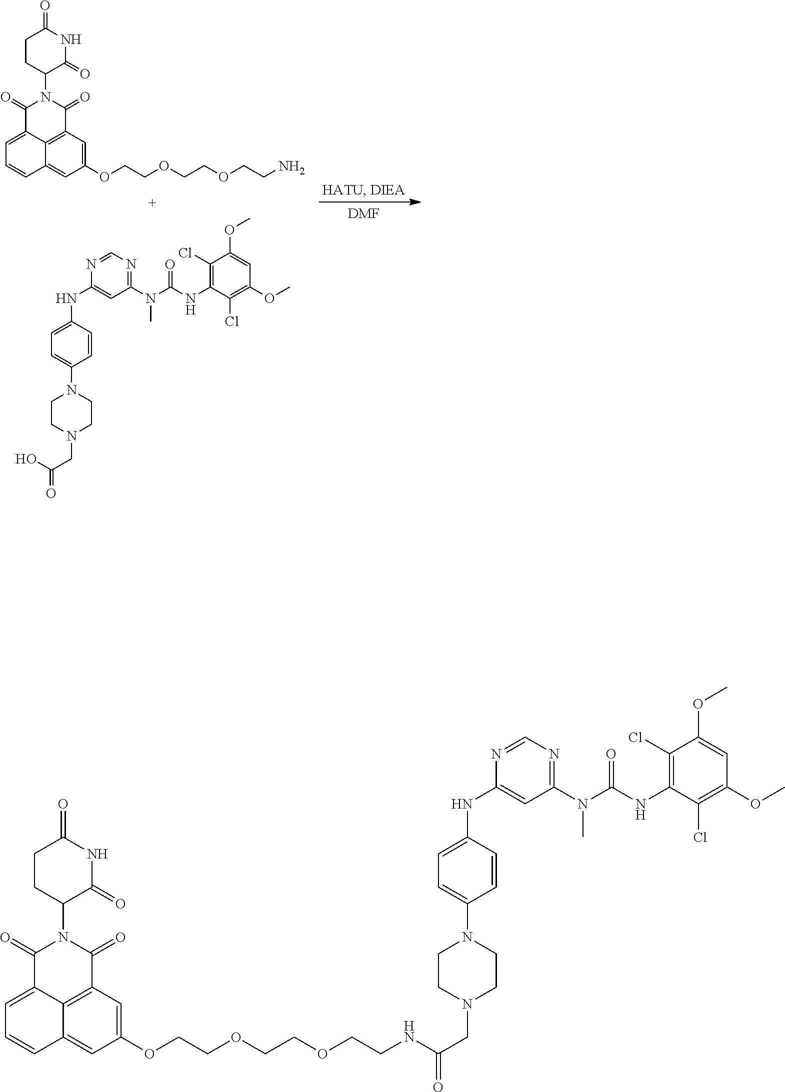

2-(4-(4-((6-(3-(2,6-Dichloro-3,5-dimethoxyphenyl)-1,3-dimethylureido)pyrimidin-4-yl)amino)phenyl)piperazin-1-yl)acetic acid (10 mg, 0.017 mmol) was added to a solution of 3-(5-(2-(2-(2-aminoethoxy)ethoxy)ethoxy)-1,3-dioxo-2,3-dihydro-1H-phenalen-2-yl)piperidine-2,6-dione TFA (9.7 mg, 0.017 mmol) in DIEA (11 mg, 0.085 mmol) and DMF (1 mL). HATU (13 mg. 0.034 mmol) was added, the reaction stirred for 30 minutes and then purified by HPLC to provide the title compound (6.7 mg, 0.0059 mmol, 35%). LC/MS m/z calculated [M+H]⁺ 1027.32, found 1027.44.

### Example 3: Synthesis of 2-(4-{4-[(6-{[(2,6-dichloro-3,5-dimethoxyphenyl)carbamoyl](methyl)amino}pyrimidin-4-yl)amino]phenyl}piperazin-1-yl)-N-(3-{[3-(2,6-dioxopiperidin-3-yl)-2,4-dioxo-3-azatricyclo[7.3.1.0^{5,13}]trideca-1(12),5,7,9(13),10-pentaen-7-yl]oxy}propyl)acetamide (2)

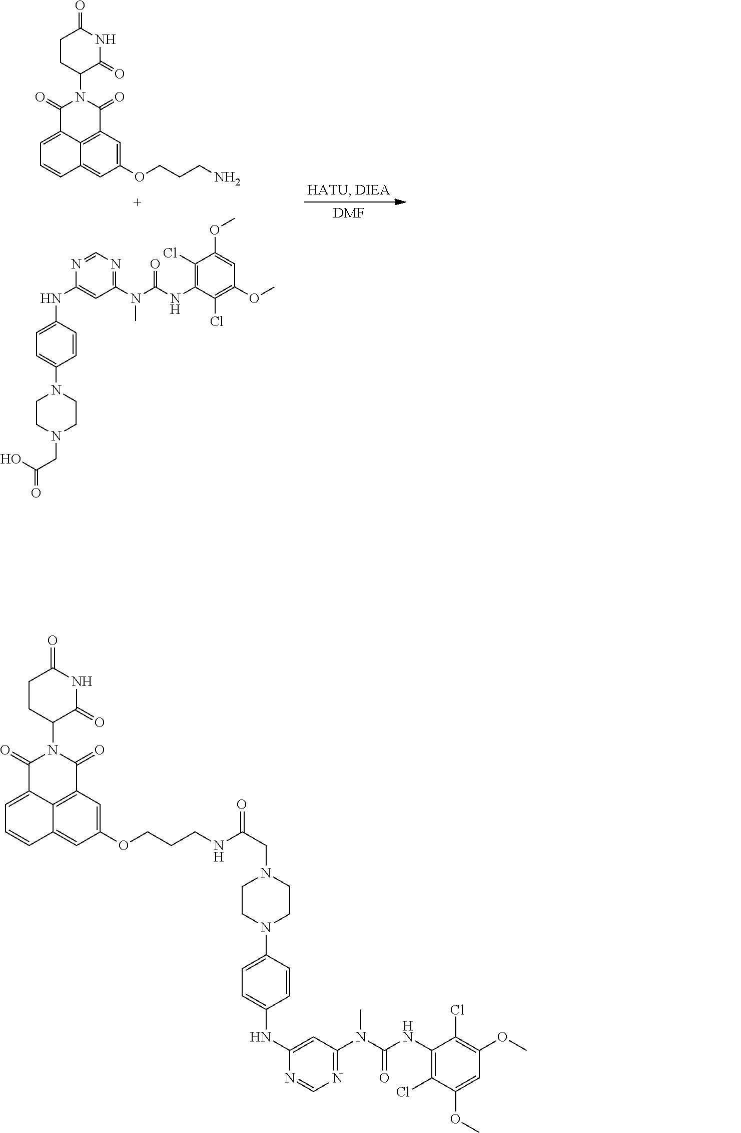

Bispecific compound 2 was synthesized in a similar manner as bispecific compound 1. LC/MS m/z calculated [M+H]⁺ 953.28, found 953.53.

### Example 4: Synthesis of 2-(4-{4-[(6-{[(2,6-dichloro-3,5-dimethoxyphenyl)carbamoyl](methyl)amino}pyrimidin-4-yl)amino]phenyl}piperazin-1-yl)-N-(6-{[3-(2,6-dioxopiperidin-3-yl)-2,4-dioxo-3-azatricyclo[7.3.1.0^{5,13}]trideca-1(12),5,7,9(13),10-pentaen-7-yl]oxy}hexyl)acetamide (3)

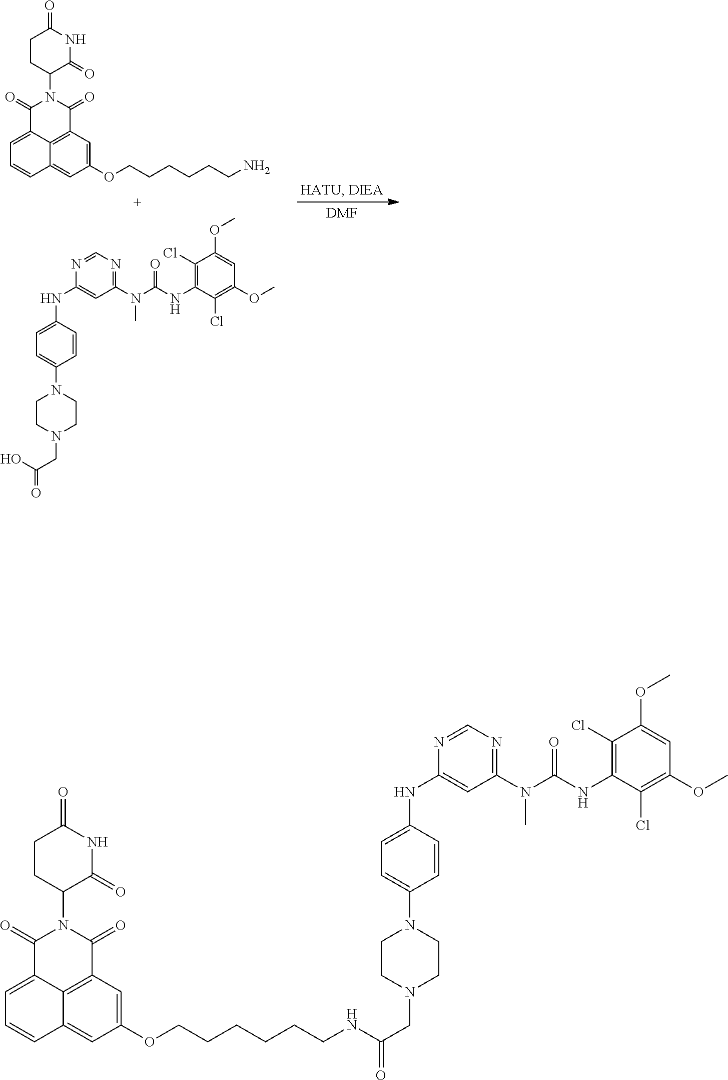

Bispecific compound 3 was synthesized in a similar manner as bispecific compound 1. LC/MS m/z calculated [M+H]⁺ 995.33, found 995.40.

### Example 5: Synthesis of 2-(4-{4-[(6-{[(2,6-dichloro-3,5-dimethoxyphenyl)carbamoyl](methyl)amino}pyrimidin-4-yl)amino]phenyl}piperazin-1-yl)-N-(2-{2-[2-(2-{[3-(2,6-dioxopiperidin-3-yl)-2,4-dioxo-3-azatricyclo[7.3.1.0^{5,13}]trideca-1(12),5,7,9(13),10-pentaen-7-yl]oxy}ethoxy)ethoxy]ethoxy}ethyl)acetamide (4)

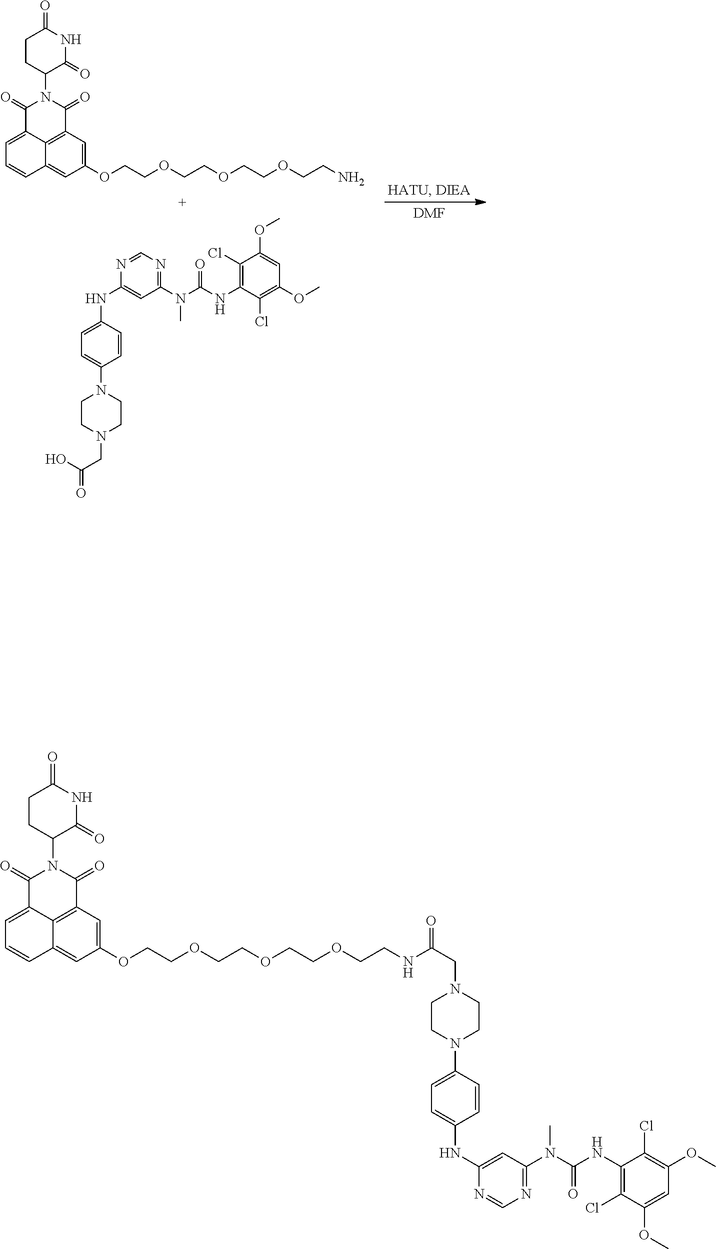

Bispecific compound 4 was synthesized in a similar manner as bispecific compound 1. LC/MS m/z calculated [M+H]⁺ 1071.35, found 1071.40.

### Example 6: Synthesis of 2-(4-{4-[(6-{[(2,6-dichloro-3,5-dimethoxyphenyl)carbamoyl](methyl)amino}pyrimidin-4-yl)amino]phenyl}piperazin-1-yl)-N-(5-{[2-(2,6-dioxopiperidin-3-yl)-1,3-dioxo-2,3-dihydro-1H-isoindol-4-yl]oxy}pentyl)acetamide (5)

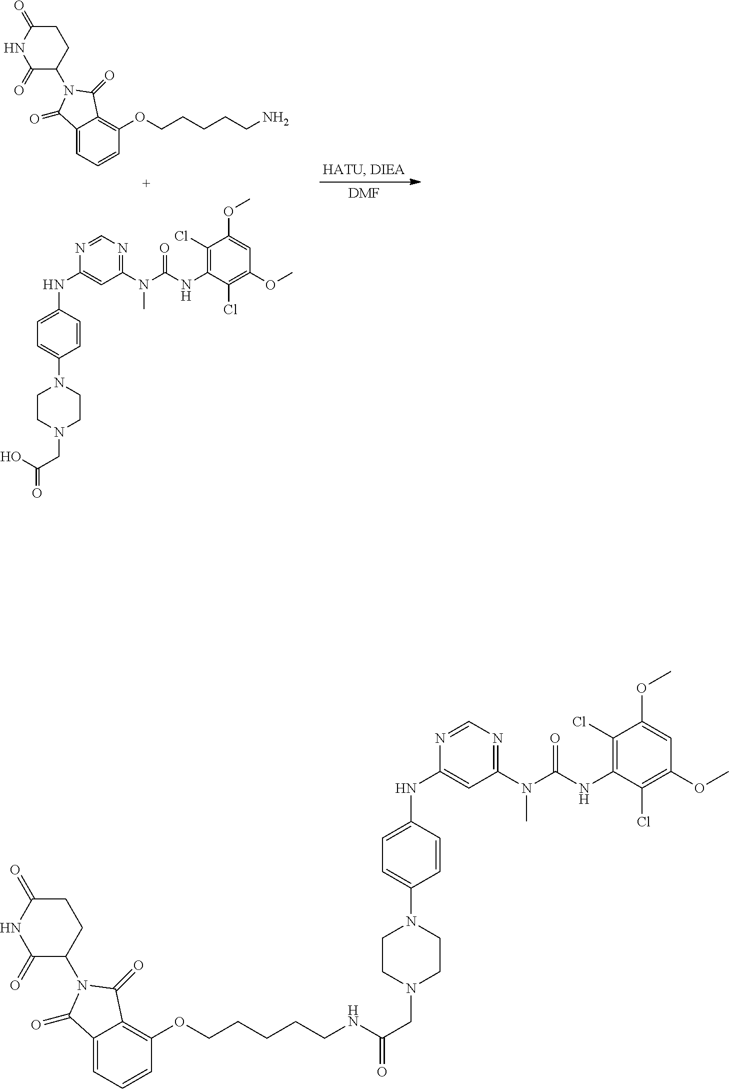

Bispecific compound 5 was synthesized in a similar manner as bispecific compound 1. LC/MS m/z calculated [M+H]⁺ 931.30, found 930.52.

### Example 7: Synthesis of (2S,4R)-1-[(2S)-2-[2-(4-{4-[(6-{[(2,6-dichloro-3,5-dimethoxyphenyl)carbamoyl](methyl)amino}pyrimidin-4-yl)amino]phenyl}piperazin-1-yl)acetamido]-3,3-dimethylbutanoyl]-4-hydroxy-N-[(1R)-1-[4-(4-methyl-1,3-thiazol-5-yl)phenyl]ethyl]pyrrolidine-2-carboxamide (6)

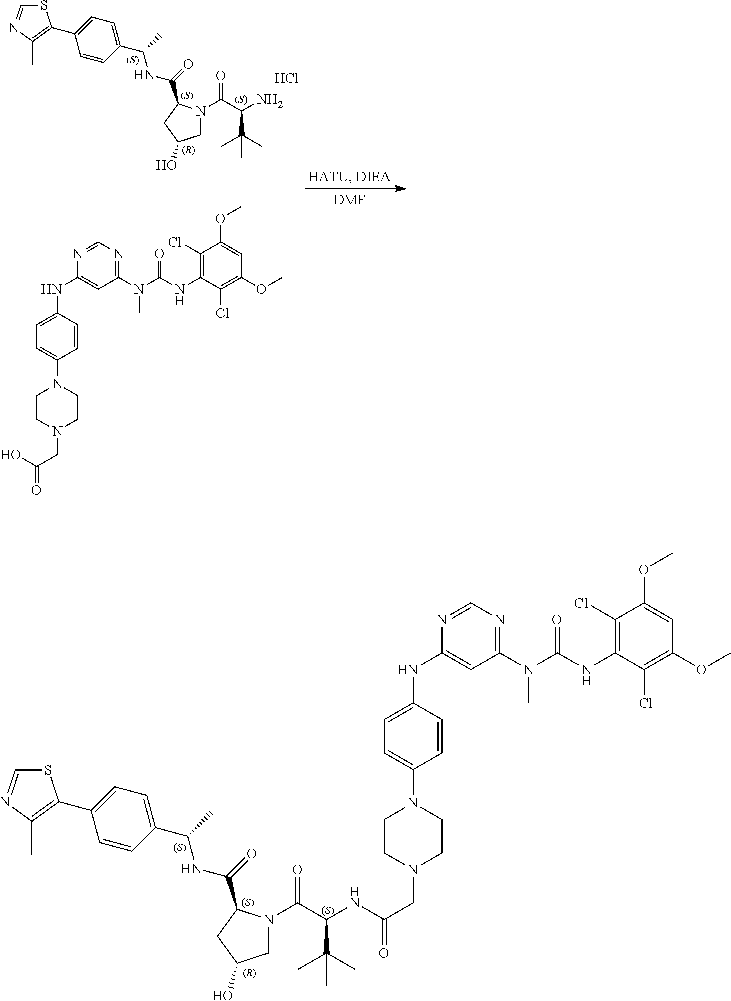

2-(4-(4-((6-(3-(2,6-Dichloro-3,5-dimethoxyphenyl)-1,3-dimethylureido)pyrimidin-4-yl)amino)phenyl)piperazin-1-yl)acetic acid (5.0 mg, 0.0085 mmol) was added to a solution of (2S,4R)-1-(L-alanyl)-4-hydroxy-N-((R)-1-(4-(4-methylthiazol-5-yl)phenyl)ethyl)pyrrolidine-2-carboxamide (4.1 mg, 0.0085 mmol) in DIEA (5.5 mg, 0.043 mmol) and DMF (1 mL). HATU was added and the reaction stirred for 30 minutes before purification by HPLC to obtain the title compound. LC/MS n z calculated [M+H]⁺ 1016.37, found 1016.43. ¹H NMR (500 MHz, DMSO-d₆) δ 11.98 (s, 1H), 10.05 (s, 1H), 9.51 (s, 1H), 8.99 (d, J=3.9 Hz, 1H), 8.77 (d, J=7.2 Hz, 1H), 8.40 (s, 2H), 7.55-7.35 (m, 5H), 7.00 (dd, J=11.1, 8.8 Hz, 2H), 6.91 (s, 1H), 6.50-6.40 (m, 2H), 5.14 (s, 1H), 4.87-4.99 (m, 1H), 4.59 (d, J=9.1 Hz, 1H), 4.44 (t, J=8.1 Hz, 2H), 4.32 (s, 1H), 4.26-4.02 (m, 2H), 3.95 (s, 9H), 3.73 (s, 2H), 3.69-3.64 (m, 2H), 3.59 (d, J=10.8 Hz, 2H), 3.12-3.04 (m, 3H), 2.47 (s, 3H), 2.11-2.00 (m, 1H), 1.89-1.77 (m, 1H), 1.39 (d, J=7.0 Hz, 3H), 0.98 (s, 9H).

### Example 8: Synthesis of (2S,4S)-1-[(2S)-2-[2-(4-{4-[(6-{[(2,6-dichloro-3,5-dimethoxyphenyl)carbamoyl](methyl)amino}pyrimidin-4-yl)amino]phenyl}piperazin-1-yl)acetamido]-3,3-dimethylbutanoyl]-4-hydroxy-N-[(1R)-1-[4-(4-methyl-1,3-thiazol-5-yl)phenyl]ethyl]pyrrolidine-2-carboxamide (7)

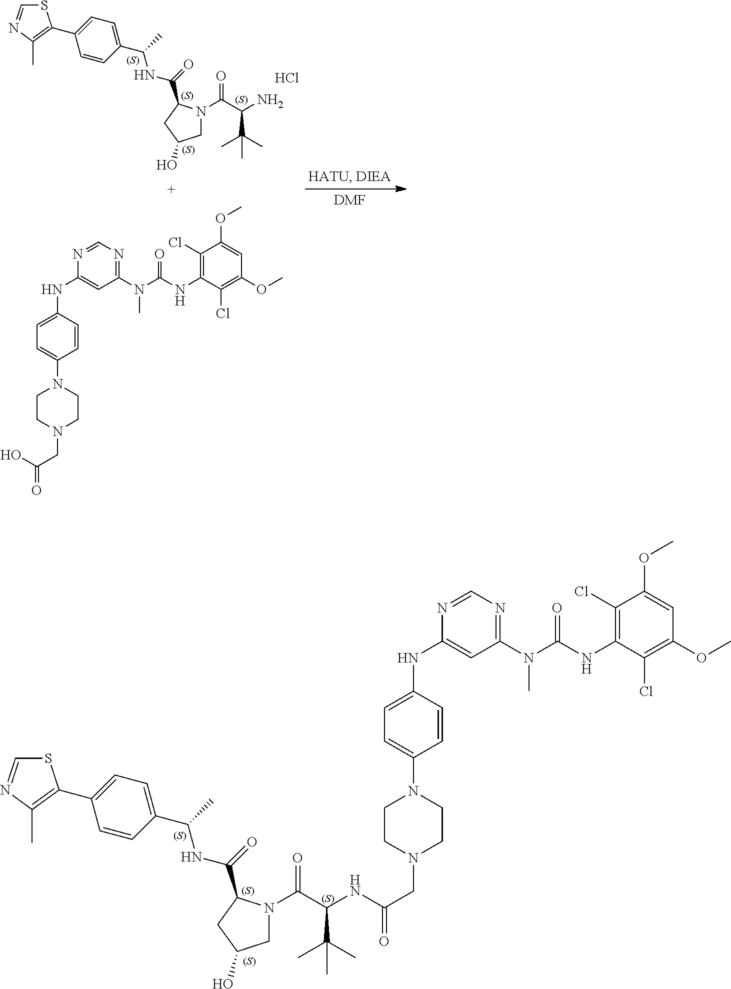

Bispecific compound 7 was synthesized in a similar manner as bispecific compound 6. LC/MS m/z calculated [M+H]⁺ 1016.37, found 1016.33. ¹H NMR (500 MHz, DMSO-d₆) δ 11.91 (s, 1H), 10.06 (s, 1H), 9.46 (s, 1H), 8.92 (s, 1H), 8.73 (d, J=9.4 Hz, 1H), 8.45-8.23 (m, 2H), 8.02 (s, 1H), 7.45-7.26 (m, 5H), 6.84 (dd, J=6.6, 2.5 Hz, 3H), 6.39 (s, 1H), 4.83 (t, J=7.3 Hz, 1H), 4.58 (d, J=10.4 Hz, 1H), 4.38 (dd, J=8.1, 6.4 Hz, 1H), 4.35-4.27 (m, 1H), 4.20-4.10 (m, 1H), 4.03 (d, J=16.3 Hz, 1H), 3.87 (s, 6H), 3.73-3.64 (m, 1H), 3.60 (s, 1H), 3.51 (dd, J=10.4, 3.3 Hz, 1H), 3.25 (s, 3H), 3.14 (s, 1H), 2.97 (s, 3H), 2.37 (d, J=4.4 Hz, 3H), 2.05-1.95 (m, 1H), 1.93-1.79 (m, 1H), 1.29 (d, J=7.0 Hz, 3H), 0.91 (s, 9H), 0.84 (t, J=7.5 Hz, 1H), 0.80 (t, J=7.0 Hz, 1H), 0.66 (s, 1H).

### Example 9: Synthesis of N-(4-{[3-(3,5-dimethoxyphenyl)-7-{[4-(4-{[(3-{[3-(2,6-dioxopiperidin-3-yl)-2,4-dioxo-3-azatricyclo[7.3.1.0^{1,13}]trideca-1(12),5(13),6,8,10-pentaen-7-yl]oxy}propyl)carbamoyl]methyl}piperazin-1-yl)phenyl]amino}-2-oxo-1H,2H,3H,4H-[1,3]diazino[4,5-d]pyrimidin-1-yl]methyl}phenyl)propenamide (8)

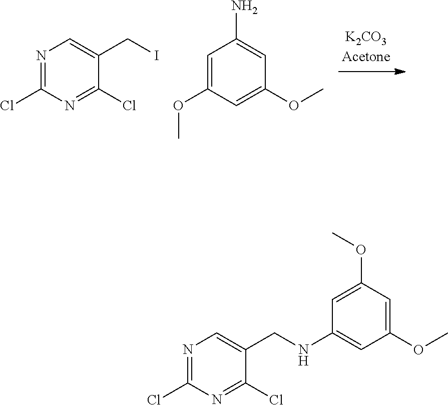 [SMILES: Clc1ncc(CI)c(Cl)n1 ; COc1cc(N)cc(OC)c1 ; COc1cc(NCc2cnc(Cl)nc2Cl)cc(OC)c1]

### N-((2,4-dichloropyrimidin-5-yl)methyl)-3,5-dimethoxyaniline

2,4-Dichloro-5-(iodomethyl)pyrimidine (200 mg, 0.69 mmol) and 3,5-dimethoxyaniline (106 mg, 0.69 mmol) were combined in acetone (3 mL) at room temperature, and K₂CO₃ (190 mg, 1.38 mmol) was added. The solvent was evaporated and the residue purified by silica gel chromatography to yield the title compound (180 mg, 0.57 mmol, 83%). LC/MS m/z calculated [M+H]⁺ 313.04, found 313.97.

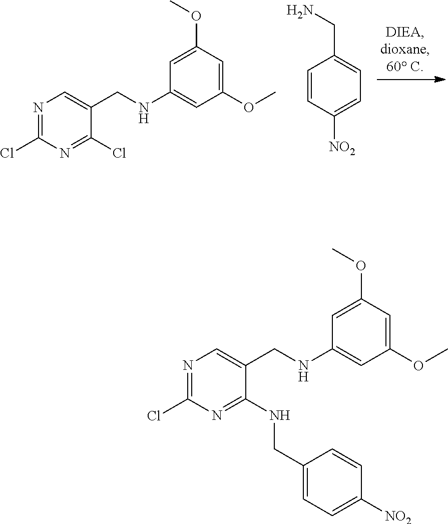

### 2-Chloro-5-(((3,5-dimethoxyphenyl)amino)methyl)-N-(4-nitrobenzyl)pyrimidin-4-amine

4-Aminomethylnitrobenzene (108 mg, 0.57 mmol) was added to N-((2,4-dichloropyrimidin-5-yl)methyl)-3,5-dimethoxyaniline (180 mg, 0.057 mmol) in DIEA (147 mg, 1.14 mmol) and dioxane (3 mL) at room temperature and stirred at 60° C. for 20 hours. The solvent was then evaporated and the residue purified by silica gel chromatography to obtain the title compound (176 mg, 0.41 mmol, 72%). LC/MS m/z calculated [M+H]⁺ 430.12, found 430.17.

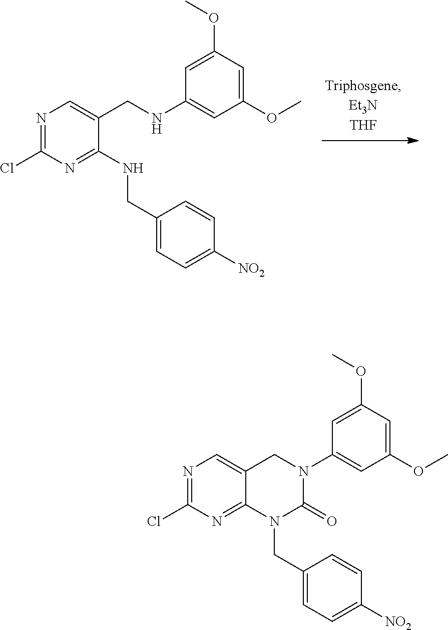 [SMILES: COc1cc(NCc2cnc(Cl)nc2NCc2ccc([N+](=O)[O-])cc2)cc(OC)c1 ; COc1cc(OC)cc(N2Cc3cnc(Cl)nc3N(Cc3ccc([N+](=O)[O-])cc3)C2=O)c1]

### 7-Chloro-3-(3,5-dimethoxyphenyl)-1-(4-nitrobenzyl)-3,4-dihydropyrimido[4,5-d]pyrimidin-2(1H)-one

2-Chloro-5-(((3,5-dimethoxyphenyl)amino)methyl)-N-(4-nitrobenzyl)pyrimidin-4-amine (176 mg, 0.41 mmol) and triphosgene (47 mg, 0.16 mmol) were dissolved in THF (2 mL). Triethylamine was slowly added at room temperature and the reaction was stirred for 1 hour. The mixture was then concentrated and purified by silica gel chromatography to yield the title compound (170 mg, 0.37 mmol, 90%). LC/MS m/z calculated [M+H]⁺ 456.10, found 456.18.

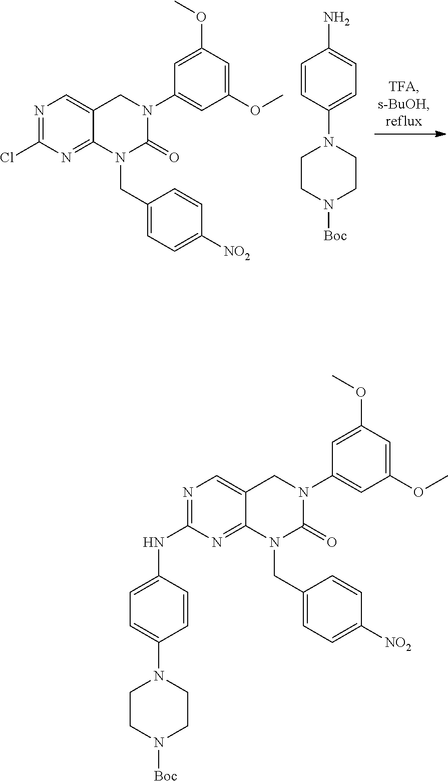

### 1-(4-Aminobenzyl)-7-chloro-3-(3,5-dimethoxyphenyl)-3,4-dihydropyrimido[4,5-d]pyrimidin-2(1H)-one

tert-Butyl 4-(4-aminophenyl)piperazine-1-carboxylate (103 mg, 0.37 mmol) and TFA (84 mg, 0.74 mmol) were added to 7-chloro-3-(3,5-dimethoxyphenyl)-1-(4-nitrobenzyl)-3,4-dihydropyrimido[4,5-d]pyrimidin-2(1H)-one in s-BuOH. The mixture was then refluxed for 16 h, the solvent evaporated, and the residue purified by silica gel chromatography to provide the title compound (192 mg, 0.28 mmol, 76%). LC/MS m/z calculated [M+H]⁺ 697.30, found 697.40.

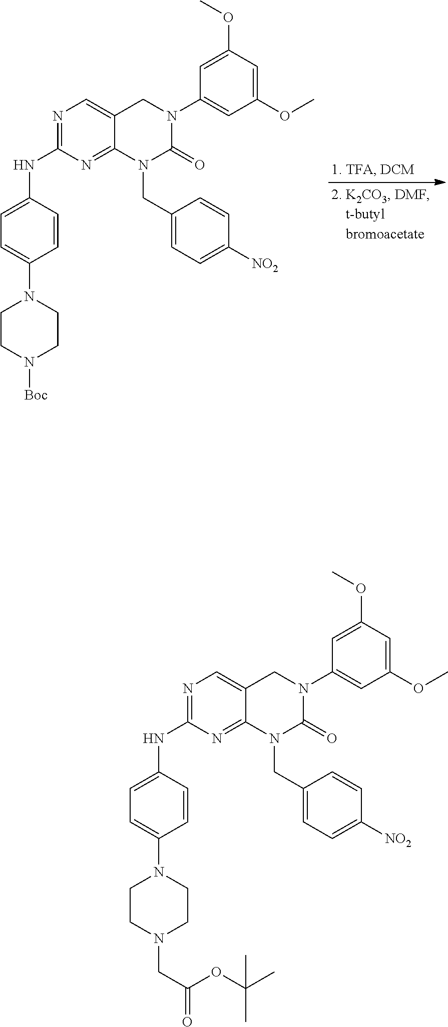 [SMILES: COc1cc(OC)cc(N2Cc3cnc(Nc4ccc(N5CCN(C(=O)OC(C)(C)C)CC5)cc4)nc3N(Cc3ccc([N+](=O)[O-])cc3)C2=O)c1 ; COc1cc(OC)cc(N2Cc3cnc(Nc4ccc(N5CCN(CC(=O)OC(C)(C)C)CC5)cc4)nc3N(Cc3ccc([N+](=O)[O-])cc3)C2=O)c1]

### 3-(3,5-Dimethoxyphenyl)-1-(4-nitrobenzyl)-7-((4-(piperazin-1-yl)phenyl)amino)-3,4-dihydropyrimido[4,5-d]pyrimidin-2(1H)-one

1-(4-Aminobenzyl)-7-chloro-3-(3,5-dimethoxyphenyl)-3,4-dihydropyrimido[4,5-d]pyrimidin-2(1H)-one (192 mg, 0.28 mmol) was dissolved in DCM (1 mL), TFA (1 mL) was added, and the solution stirred at room temperature for 1 hour. The solvent was then removed to yield the amine. LC/MS m/z calculated [M+H]⁺ 597.25, found 597.32.

This intermediate was then dissolved in DMF (1 mL), treated with K₂CO₃ (116 mg, 0.84 mmol) and t-butyl bromoacetate (55 mg, 0.28 mmol) was added. The mixture was stirred at 50° C. for 14 h and purified by silica gel chromatography to obtain the title compound (127 mg, 0.18 mmol, 64% over 2 steps). LC/MS m/z calculated [M+H]⁺ 711.32, found 711.44.

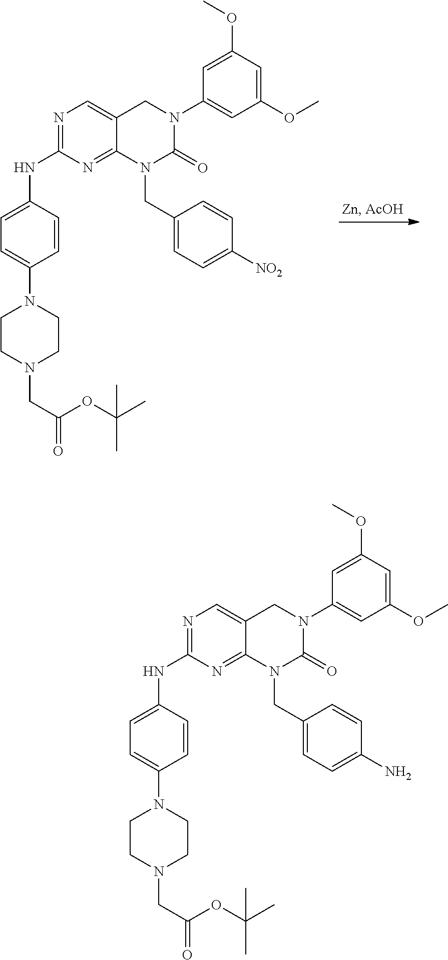 [SMILES: COc1cc(OC)cc(N2Cc3cnc(Nc4ccc(N5CCN(CC(=O)OC(C)(C)C)CC5)cc4)nc3N(Cc3ccc([N+](=O)[O-])cc3)C2=O)c1 ; COc1cc(OC)cc(N2Cc3cnc(Nc4ccc(N5CCN(CC(=O)OC(C)(C)C)CC5)cc4)nc3N(Cc3ccc(N)cc3)C2=O)c1]

### tert-Butyl 2-(4-(4-((8-(4-aminobenzyl)-6-(3,5-dimethoxyphenyl)-7-oxo-5,6,7,8-tetrahydropyrimido[4,5-d]pyrimidin-2-yl)amino)phenyl)piperazin-1-yl)acetate

tert-Butyl 2-(4-(4-((6-(3,5-dimethoxyphenyl)-8-(4-nitrobenzyl)-7-oxo-5,6,7,8-tetrahydropyrimido[4,5-d]pyrimidin-2-yl)amino)phenyl)piperazin-1-yl)acetate (127 mg, 0.18 mmol) was dissolved in acetic acid (2 mL) and Zn was added at room temperature. The suspension was stirred for 3 hours before being filtered, concentrated and purified by silica gel chromatography to provide the title compound (77 mg, 0.11 mmol, 63%). LC/MS m/z calculated [M+H]⁺ 681,34, found 681,40.

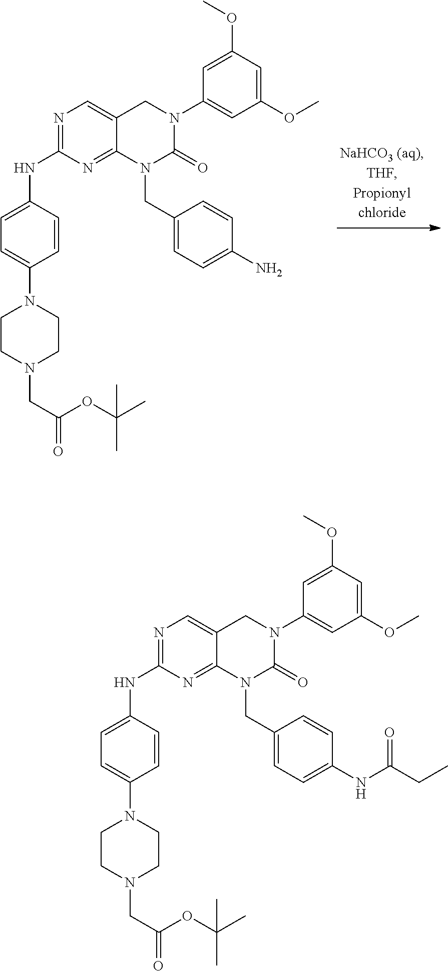 [SMILES: COc1cc(OC)cc(N2Cc3cnc(Nc4ccc(N5CCN(CC(=O)OC(C)(C)C)CC5)cc4)nc3N(Cc3ccc(N)cc3)C2=O)c1 ; CCC(=O)Nc1ccc(CN2C(=O)N(c3cc(OC)cc(OC)c3)Cc3cnc(Nc4ccc(N5CCN(CC(=O)OC(C)(C)C)CC5)cc4)nc32)cc1]

### tert-Butyl 2-(4-(4-((6-(3,5-dimethoxyphenyl)-7-oxo-8-(4-propionamidobenzyl)-5,6,7,8-tetrahydropyrimido[4,5-d]pyrimidin-2-yl)amino)phenyl)piperazin-1-yl)acetate

tert-Butyl 2-(4-(4-((8-(4-aminobenzyl)-6-(3,5-dimethoxyphenyl)-7-oxo-5,6,7,8-tetrahydropyrimido[4,5-d]pyrimidin-2-yl)amino)phenyl)piperazin-1-yl)acetate (77 mg, 0.11 mmol) was dissolved in THF (1 mL) and treated with a saturated NaHCO₃ solution. At 0° C., propionyl chloride (12 mg, 0.13 mmol) was added and after 5 minutes of stirring, water was added and the mixture extracted with EtOAc. Combined extracts were washed with brine, dried with Na₂SO₄ and concentrated to yield the title compound (23 mg, 0.031 mmol, 28%). LC/MS m/z calculated [M+H]⁺ 737.37, found 736.91.

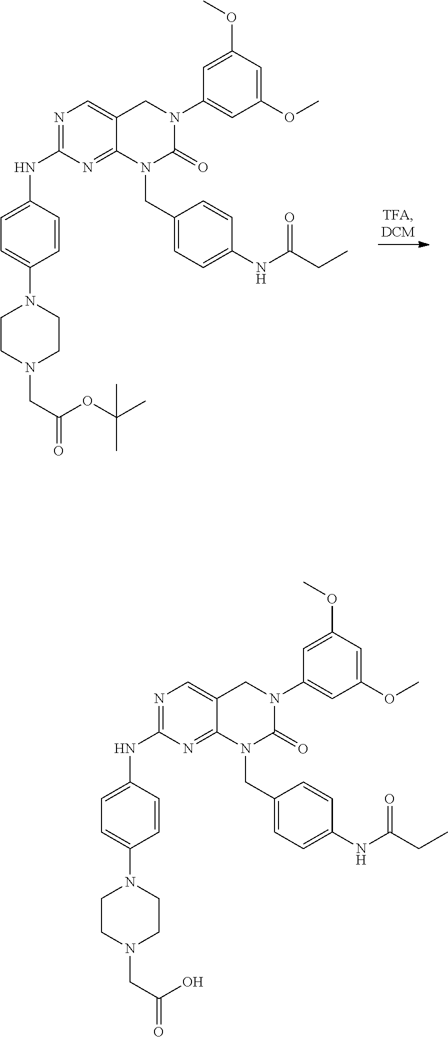 [SMILES: CCC(=O)Nc1ccc(CN2C(=O)N(c3cc(OC)cc(OC)c3)Cc3cnc(Nc4ccc(N5CCN(CC(=O)OC(C)(C)C)CC5)cc4)nc32)cc1 ; CCC(=O)Nc1ccc(CN2C(=O)N(c3cc(OC)cc(OC)c3)Cc3cnc(Nc4ccc(N5CCN(CC(=O)O)CC5)cc4)nc32)cc1]

### 2-(4-(4-((6-(3,5-Dimethoxyphenyl)-7-oxo-8-(4-propionamidobenzyl)-5,6,7,8-tetrahydropyrimido[4,5-d]pyrimidin-2-yl)amino)phenyl)piperazin-1-yl)acetic acid

tert-Butyl 2-(4-(4-((6-(3,5-dimethoxyphenyl)-7-oxo-8-(4-propionamidobenzyl)-5,6,7,8-tetrahydropyrimido[4,5-d]pyrimidin-2-yl)amino)phenyl)piperazin-1-yl)acetate (23 mg, 0.031) was dissolved in DCM (1 mL), TFA (1 mL) was added, and the solution stirred at room temperature for 1 hour. The solvent was then removed to provide the title compound. LC/MS m/z calculated [M+H]⁺ 681,31, found 681,90.

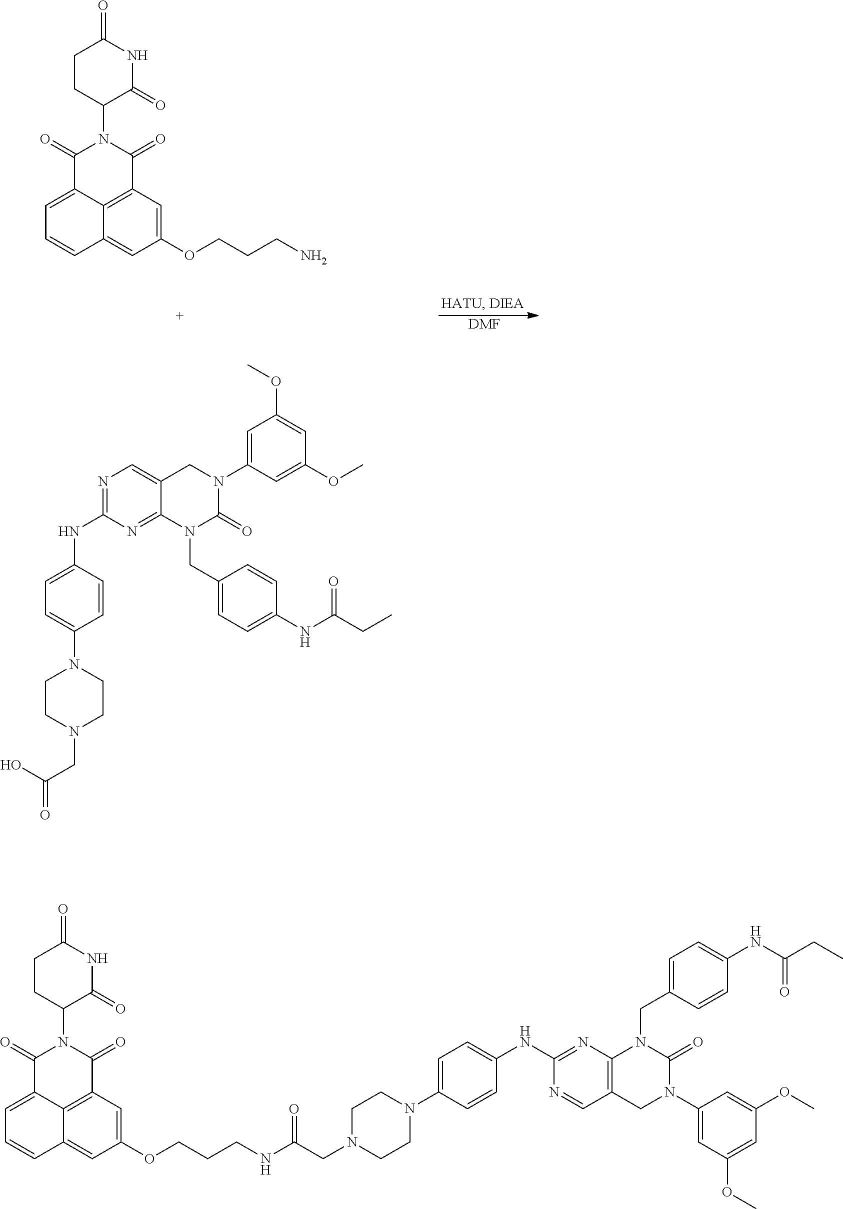

2-(4-(4-((6-(3,5-Dimethoxyphenyl)-7-oxo-8-(4-propionamidobenzyl)-5,6,7,8-tetrahydropyrimido[4,5-d]pyrimidin-2-yl)amino)phenyl)piperazin-1-yl)acetic acid (7.0 mg, 0.010 mmol) and 3-(5-(3-aminopropoxy)-1,3-dioxo-2,3-dihydro-1H-phenalen-2-yl)piperidine-2,6-dione TFA (3.8 mg, 0.010 mmol) were combined in DMF (1 mL) and DIEA (6.6 mg, 0.052 mmol). At room temperature, HATU (7.8 mg, 0.021 mmol) was added. The solution was stirred for 30 minutes, then purified by HPLC to provide bispecific compound 8 (3.3 mg, 0.0031 mmol, 31%). LC/MS m/z calculated [M+H]⁺ 1044.43, found 1043.64.

### Example 10: Synthesis of N-(4-{[3-(3,5-dimethoxyphenyl)-7-{[4-(4-{[(5-{[2-(2,6-dioxopiperidin-3-yl)-1,3-dioxo-2,3-dihydro-1H-isoindol-4-yl]oxy}pentyl)carbamoyl]methyl}piperazin-1-yl)phenyl]amino}-2-oxo-1H,2H,3H,4H-[1,3]diazino[4,5-d]pyrimidin-1-yl]methyl}phenyl)propenamide (9)

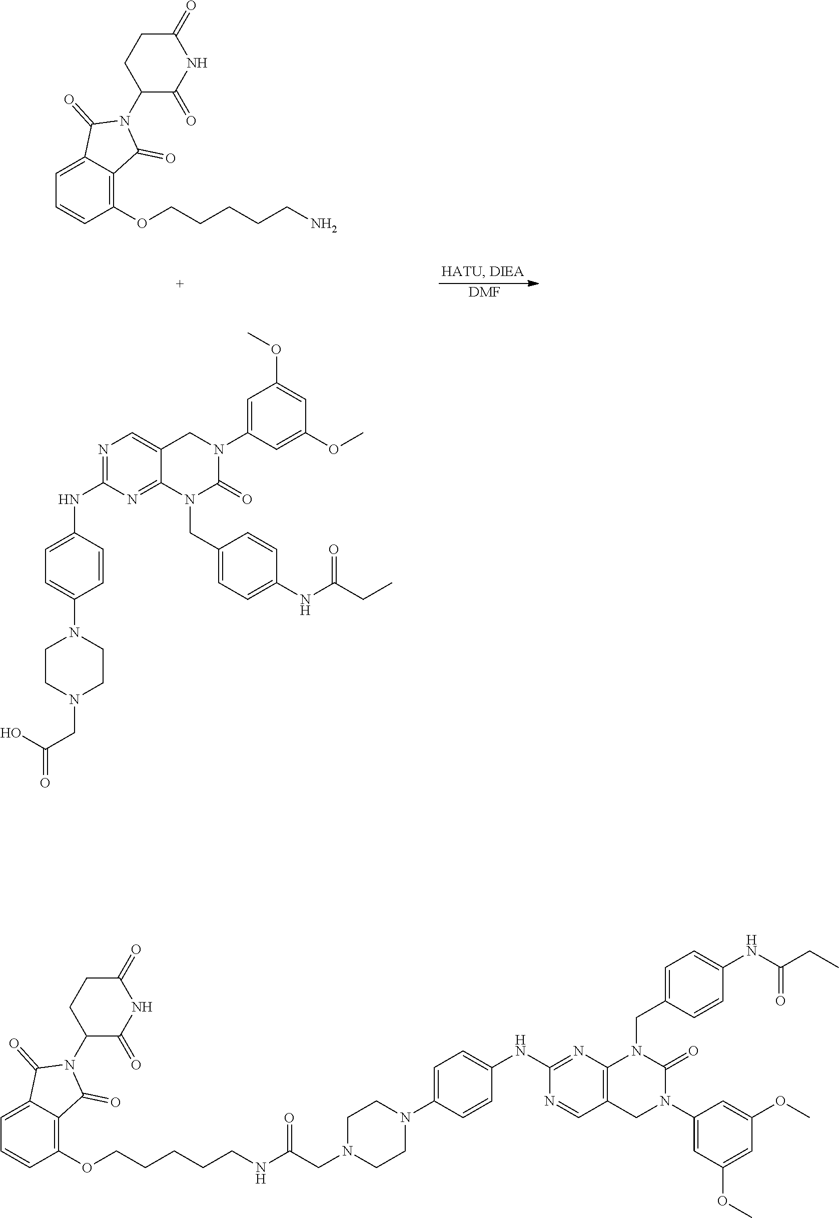

Bispecific compound 9 was synthesized in a similar manner as bispecific compound 8. LC/MS m/z calculated [M+H]⁺ 1022.44, found 1021.63.

### Example 11: Synthesis of N-(4-{[3-(3,5-dimethoxyphenyl)-7-[(4-{4-[({2-[2-(2-{[3-(2,6-dioxopiperidin-3-yl)-2,4-dioxo-3-azatricyclo[7.3.1.0^{5,13}]trideca-1(12),5(13),6,8,10-pentaen-7-yl]oxy}ethoxy]ethoxy]ethyl}carbamoyl)methyl]piperazin-1-yl}phenyl)aminol-2-oxo-1H,2H,3H,4H-[1,3]diazino[4,5-d]pyrimidin-1-yl]methyl}phenyl)propenamide (10)

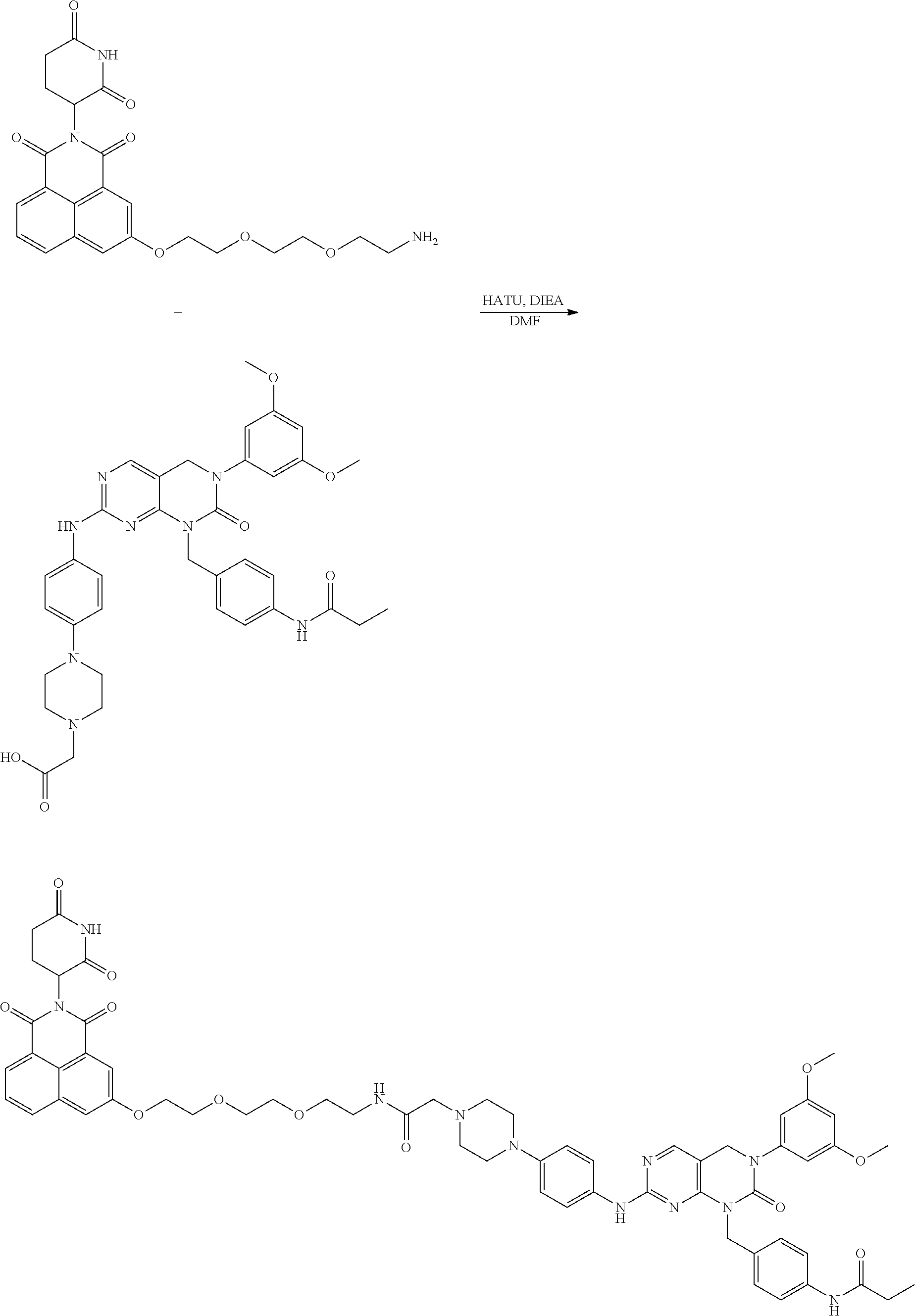

Bispecific compound 10 was synthesized in a similar manner as bispecific compound 8. LC/MS m/z calculated [M+H]⁺ 1118.47, found 1117.56.

### Example 12: Synthesis of N-[4-({7-[(tert-butylcarbamoyl)amino]-6-(3,5-dimethoxyphenyl]pyrido[2,3-d]pyrimidin-2-yl}amino)butyl-2-{[2-(2,6-dioxopiperidin-3-v-1,3-dioxo-2,3-dihydro-1H-isoindol-4-yl]oxy}acetamide (11)

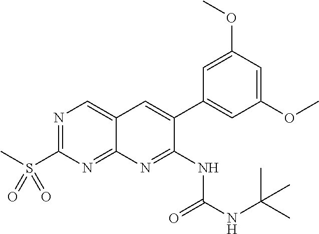

### 1-(tert-Butyl)-3-(6-(3,5-dimethoxyphenyl)-2-(methylsulfonyl)pyrido [2,3-d]pyrimidin-7-yl)urea

This compound was synthesized using the procedure described in Thompson et al. J. Med. Chem. 48:4628-4653 (2005).

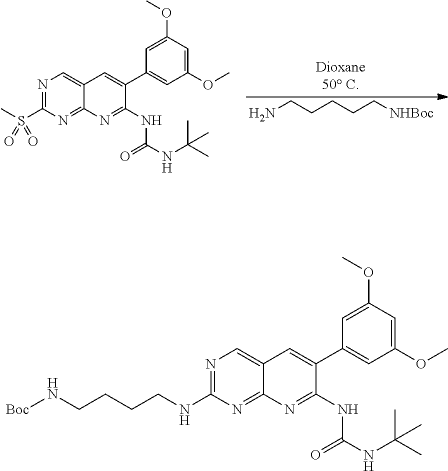

### tert-Butyl (4-((7-(3-(tert-butyl)ureido)-6-(3,5-dimethoxyphenyl)pyrido[2,3-d]pyrimidin-2-yl)amino)butyl)carbamate

tert-Butyl (5-aminopentyl)carbamate (101 mg, 0.54 mmol) was added to 1-(tert-butyl)-3-(6-(3,5-dimethoxyphenyl)-2-(methylsulfonyl)pyrido[2,3-d]pyrimidin-7-yl)urea (223 mg, 0.49 mmol) and stirred for 6 hours at 50° C. The solvent was the evaporated and the crude residue purified by silica gel chromatography to provide the title compound (174 mg, 0.31 mmol, 63%). LC/MS m/z calculated [M+H]⁺ 568.32, found 568.40.

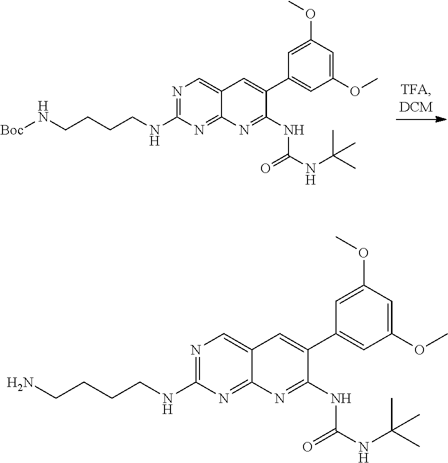 [SMILES: COc1cc(OC)cc(-c2cc3cnc(NCCCCNC(=O)OC(C)(C)C)nc3nc2NC(=O)NC(C)(C)C)c1 ; COc1cc(OC)cc(-c2cc3cnc(NCCCCN)nc3nc2NC(=O)NC(C)(C)C)c1]

### 1-(2-((4-Aminobutyl)amino)-6-(3,5-dimethoxyphenyl)pyrido[2,3-d]pyrimidin-7-yl)-3-(tert-butyl)urea

tert-Butyl (4-((7-(3-(tert-butyl)ureido)-6-(3,5-dimethoxyphenyl)pyrido[2,3-d]pyrimidin-2-yl)amino)butyl)carbamate (174 mg, 0.31 mmol) was dissolved in DCM (1 mL) and TFA (1 mL) was added, and the solution stirred for 2 hours. The solvent was removed and to obtain the title compound as a TFA salt. LC/MS m/z calculated [M+H]⁺ 468.26, found 468.10.

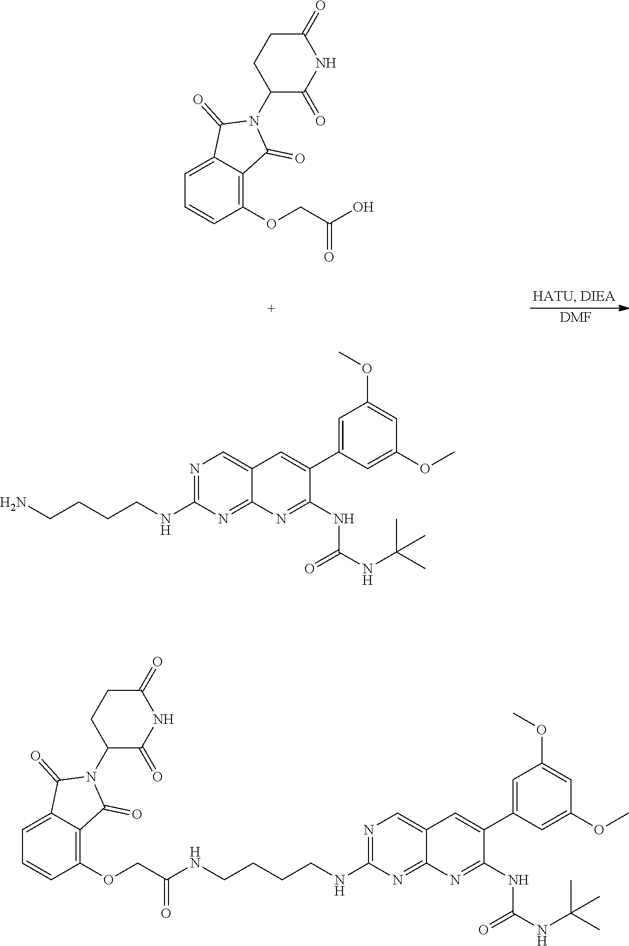

1-(2-((4-Aminobutyl)amino)-6-(3,5-dimethoxyphenyl)pyrido[2,3-d]pyrimidin-7-yl)-3-(tert-butyl)urea TFA (20 mg, 0.034 mmol) and 2-((2-(2,6-dioxopiperidin-3-yl)-1,3-dioxoisoindolin-4-yl)oxy)acetic acid (11 mg, 0.034 mmol) were dissolved in a solution of DIEA (30 μL, 0.17 mmol) and DMF (1 mL). HATU (26 mg, 0.068 mmol) was added and the reaction stirred for 30 minutes before being purified by HPLC to obtain bispecific compound 11 (16.8 mg, 0.021 mmol, 63%). LC/MS m/z calculated [M+H]⁺ 782.32, found 782.30. ¹H NMR (500 MHz, DMSO-d₆) δ 11.04 (s, 1H), 8.93 (s, 1H), 8.85 (s, 1H), 7.98 (s, 2H), 7.91 (t, J=5.8 Hz, 1H), 7.83 (s, 1H), 7.72 (t, J=7.9 Hz, 1H), 7.40 (d, J=7.2 Hz, 1H), 7.32 (d, J=8.6 Hz, 1H), 6.60-6.53 (m, 3H). 5.04 (dd, z J=12.8, 5.4 Hz, 1H), 4.70 (s, 2H), 3.74 (s. 6H), 3.33 (d, J=6.5 Hz, 2H), 3.15 (d, J=7.9 Hz, 2H), 2.88-2.77 (m, 1H), 2.56-2.46 (m, 2H), 2.00-1.93 (m, 1H), 1.56 (s, 2H), 1.45 (s, 2H), 1.29 (s, 9H).

### Example 13: Synthesis of N-[4-({7-[(tert-butylcarbamoyl)amino]-6-(3,5-dimethoxyphenyl)pyrido[2,3-d]pyrimidin-2-yl}amino)butyl]-2-{[3-(2,6-dioxopiperidin-3-yl)-2,4-dioxo-3-azatricyclo[7.3.1.0^{5,13}]trideca-1(12),5,7,9(13),10-pentaen-7-yl]oxy}acetamide (12)

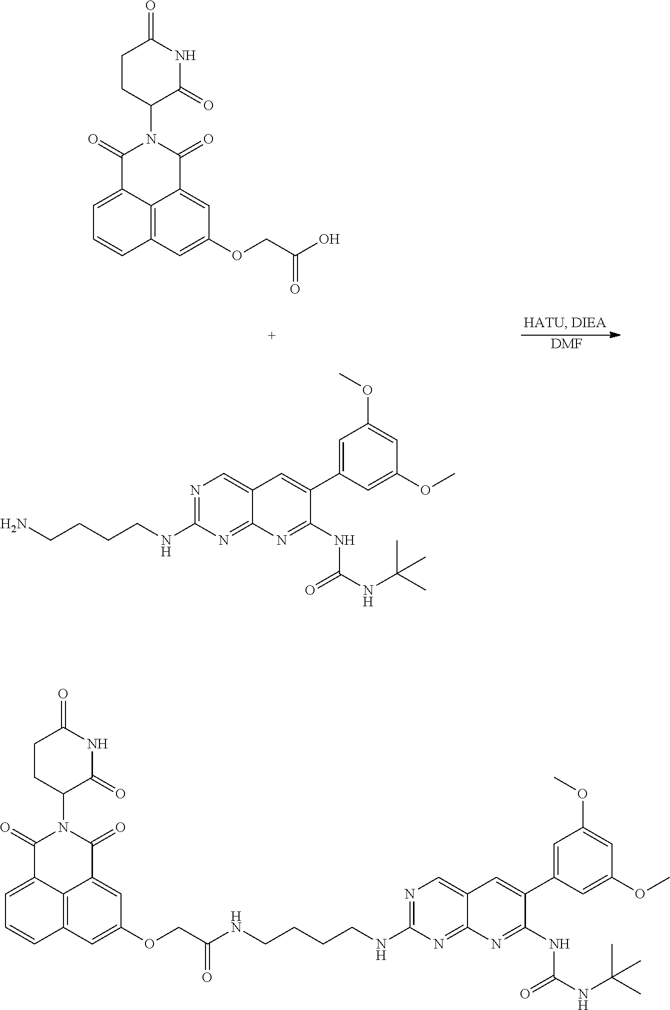

Bispecific compound 12 was synthesized in a similar manner as bispecific compound 11. LC/MS m/z calculated [M+H]⁺ 832.33, found 832.4. ¹H NMR (500 MHz, DMSO-d₆) δ 10.95 (s, 1H), 10.23 (d, J=13.5 Hz, 1H), 8.97-8.73 (m, 1H), 8.41-8.16 (m, 3H), 8.02 (s, 1H), 7.96-7.83 (m, 2H), 7.76 (q, J=7.8 Hz, 1H), 7.71 (s, 1H), 7.02 (s, 1H), 6.61-6.44 (m, 3H), 5.76 (dt, J=11.3, 5.2 Hz, 1H), 4.66 (s, 2H), 3.73 (s, 6H), 3.32 (t, J=6.5 Hz, 2H), 3.16 (s, 2H), 2.95-2.78 (m, 1H), 2.61-2.46 (m, 2H), 1.97 (td, J=11.8, 9.4, 5.0 Hz, 1H), 1.64-1.39 (m, 4H), 1.28 (d, J=4.1 Hz, 9H).

### Example 14: Synthesis of N-[4-({7-[(tert-butylcarbamoyl)aminol-6-(3,5-dimethoxyphenyl)pyrido[2,3-d]pyrimidin-2-yl}amino)butyl]-9-f[2-(2,6-dioxopiperidin-3-yl)-1,3-dioxo-2,3-dihydro-1H-isoindol-4-yl]oxy}nonanamide (13)

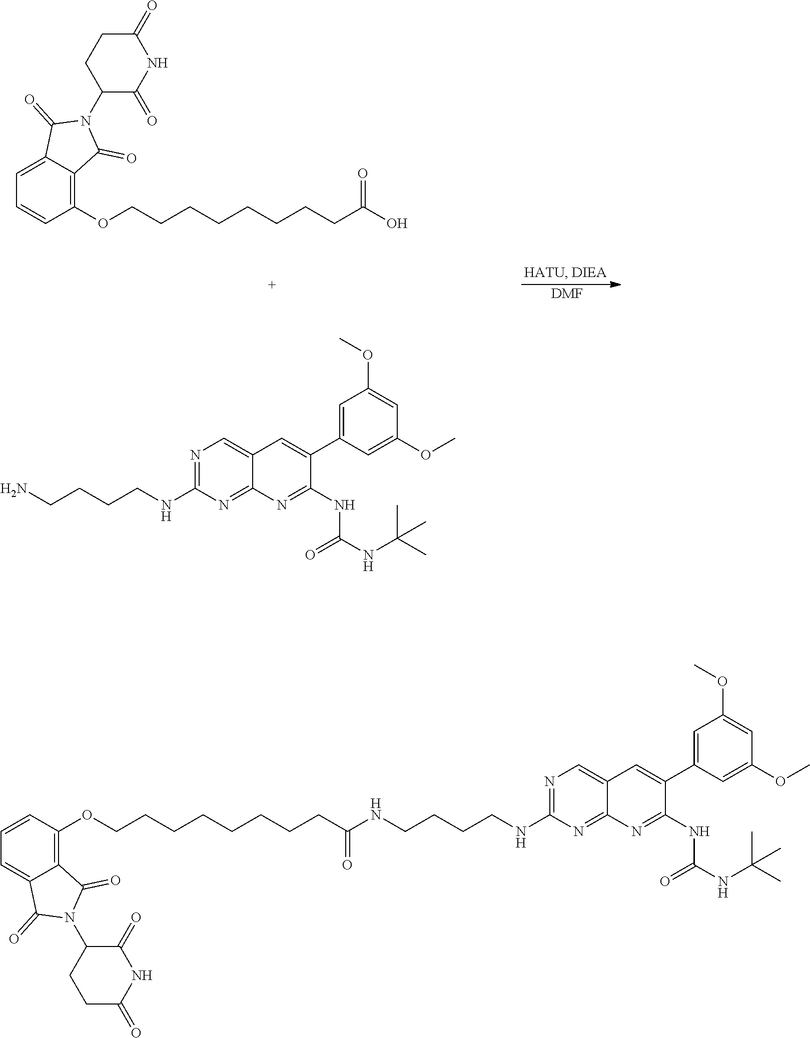

Bispecific compound 13 was synthesized in a similar manner as bispecific compound 11. LC/MS m/z calculated [M+H]⁺ 880.43, found 880.50. ¹H NMR (500 MHz, DMSO-d₆) δ 11.03 (s, 1H), 8.96 (s, 1H), 8.87 (s, 1H), 8.03 (s, 1H), 7.89 (s, ill), 7.72 (dd, J=8.5, 7.2 Hz, 1H), 7.67 (s, 1H), 7.42 (d, J=8.5 Hz, 1H), 7.36 (d, J=7.2 Hz, 1H), 6.71-6.34 (in, 311), 4.99 (d, J=5.4 Hz, 1H), 4.11 (t, J=6.4 Hz, 2H), 3.73 (s, 6H), 3.35-3.29 (m, 3H), 3.07-2.91 (m, 2H), 2.86-2.73 (m, 1H), 2.59-2.45 (m, 1H), 1.96 (dd, J=8.9, 6.1 Hz, 3H), 1.72-1.59 (m, 2H), 1.59-1.45 (m, 2H), 1.45-1.32 (m, 6H), 1.30 (s, 9H), 1.26-1.08 (in, 7H).

### Example 15: Synthesis of (2S,4R)-1-[(2S)-2-{3-[2-(4-{4-[(6-{[(2,6-dichloro-3,5-dimethoxyphenyl)carbamoyl](methyl)amino}pyrimidin-4-yl)amino]phenyl}piperazin-1-yl)acetamido]propanamido}-3,3-dimethylbutanoyl]-4-hydroxy-N-[(1R)-1-[4-(4-methyl-1,3-thiazol-5-yl)phenyl]ethyl]pyrrolidine-2-carboxamide (14)

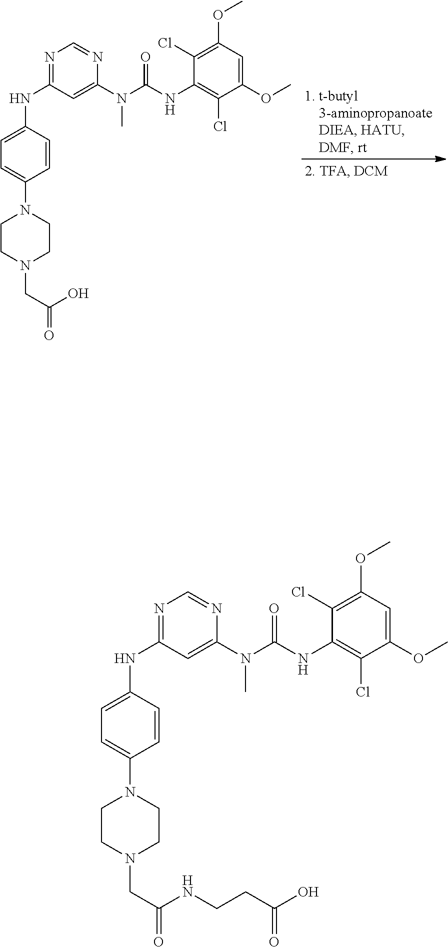 [SMILES: COc1cc(OC)c(Cl)c(NC(=O)N(C)c2cc(Nc3ccc(N4CCN(CC(=O)O)CC4)cc3)ncn2)c1Cl ; COc1cc(OC)c(Cl)c(NC(=O)N(C)c2cc(Nc3ccc(N4CCN(CC(=O)NCCC(=O)O)CC4)cc3)ncn2)c1Cl]

### 3-(2-(4-(4-((6-(3-(2,6-dichloro-3,5-dimethoxyphenyl)-1-methylureido)pyrimidin-4-yl)amino)phenyl)piperazin-1-yl)acetamido)propanoic acid

2-(4-(4-((6-(3-(2,6-dichloro-3,5-dimethoxyphenyl)-1-methylureido)pyrimidin-4-yl)amino)phenyl)piperazin-1-yl)acetic acid (11 mg, 0.025 mmol) and tert-butyl 3-aminopropanoate hydrochloride (5 mg, 0.025 mmol) were dissolved in DMF (1 mL). DIEA (20 μL, 0.125 mmol) was added, followed by HATU (19 mg, 0.050 mmol) and the reaction was stirred for 20 minutes. Water was added and the mixture was extracted with ethyl acetate. Combined organics were washed with brine, dried over Na₂SO₄, concentrated, and the crude residue was purified by silica gel chromatography to provide the tert-butyl ester. LC/MS found 716.70. This ester was dissolved in DCM (1 mL) and TFA (1 mL) was added, and the solution stirred at room temperature for 2 hours. The solvent was removed under pressure to provide the title compound (10.6 mg, 0.016 mmol, 64%). LC/MS m/z calculated for [M+H]⁺ 661.20, found 660.70.

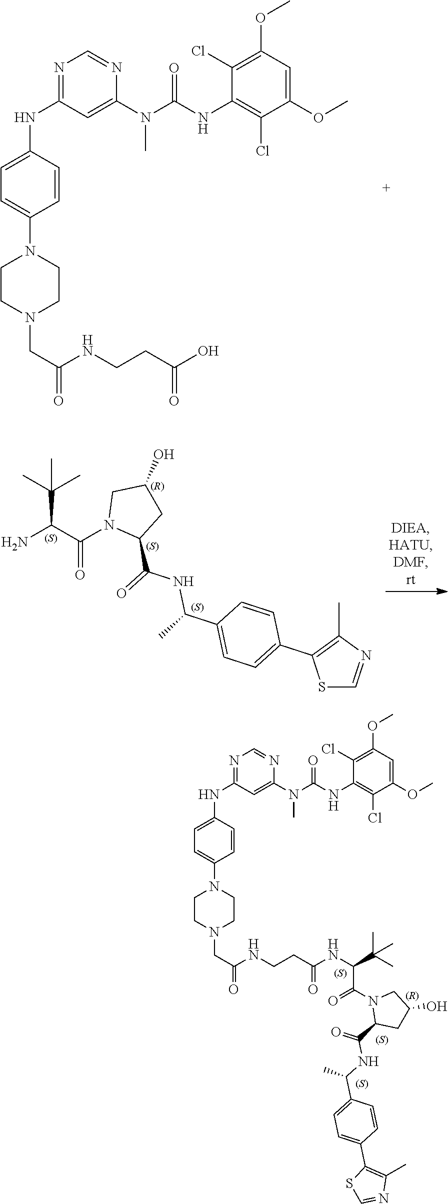

3-(2-(4-(4-((6-(3-(2,6-dichloro-3,5-dimethoxyphenyl)-1-methylureido)pyrimidin-4-yl)amino)phenyl)piperazin-1-yl)acetamido)propanoic acid (10.6 mg, 0.016 mmol) and (1R,4S)-2-((S)-2-amino-3,3-dimethylbutanoyl)-4-hydroxy-N-((R)-1-(4-(4-methylthiazol-5-yl)phenyl)ethyl)cyclopentane-1-carboxamide dihydrochloride (7.7 mg, 0.016 mmol) were dissolved in DMF (1 mL). DIEA (14 μL, 0.080 mmol) was added followed by HATU (12 mg, 0.032 mmol) and the reaction was stirred for 20 minutes before being purified by HPLC to provide bispecific compound 14 as a white solid and TFA salt (7.8 mg, 0.032 mmol, 45%). LC/MS m/z calculated for [M+H]⁺ 1087.41, found 1087.60. ¹H NMR (500 MHz, DMSO-d₆) δ 11.91 (s, 1H), 10.04 (s, 1H), 9.46 (s, 1H), 8.92 (s, 1H), 8.53 (s, 1H). 8.33 (s, 1H), 8.29 (d, J=7.8 Hz, 1H), 7.92 (d, J=9.2 Hz, 1H), 7.41 (d, J=8.5 Hz, 2H), 7.37 (d, J=8.3 Hz, 2H), 7.31 (d, J=8.4 Hz, 2H), 6.92 (d, J=9.3 Hz, 2H), 6.84 (s, 1H), 6.39 (s, 1H), 5.06 (s, 1H). 4.85 (s, 1H), 4.47 (d, J=9.3 Hz, 1H), 4.35 (s, 1H), 4.26-4.20 (m, 1H), 3.89 (s, 2H), 3.87 (s, 6H), 3.65 (d, J=2.3 Hz, 2H), 3.59-3.49 (m, 3H), 3.49-3.39 (m, 3H), 3.29 (q, J=6.7 Hz, 2H), 3.25 (s, 3H), 3.20 (s, 2H), 3.10-2.95 (m, 2H), 2.39 (s, 3H), 1.96 (t, J=10.5 Hz, 1H), 1.74 (s, 1H), 1.31 (d, J=7.0 Hz, 3H), 0.88 (s, 9H).

### Example 16: Synthesis of (2S,4R)-1-[(2S)-2-(3-{2-[2-(4-{4-[(6-{[(2,6-dichloro-3,5-dimethoxyphenyl)carbamoyl](methyl)amino}pyrimidin-4-yl)amino]phenyl}piperazin-1-yl)acetamido]ethoxy}propanamido)-3,3-dimethylbutanoyl]-4-hydroxy-N-[(1R)-1-[4-(4-methyl-1,3-thiazol-5-yl)phenyl]ethyl]pyrrolidine-2-carboxamide (15)

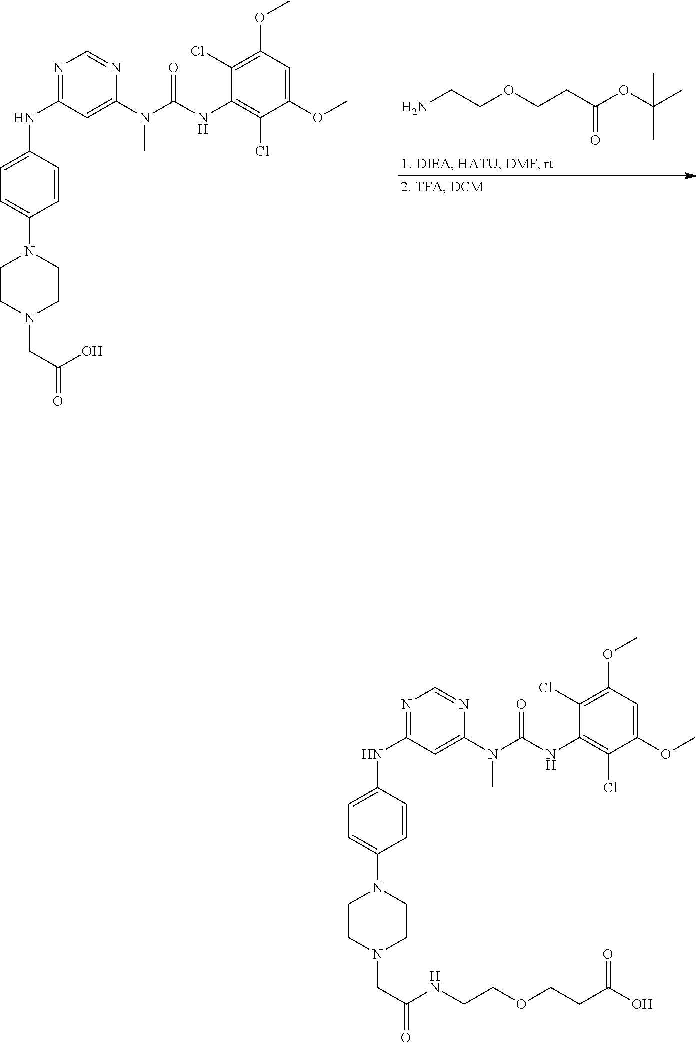 [SMILES: COc1cc(OC)c(Cl)c(NC(=O)N(C)c2cc(Nc3ccc(N4CCN(CC(=O)O)CC4)cc3)ncn2)c1Cl ; CC(C)(C)OC(=O)CCOCCN ; COc1cc(OC)c(Cl)c(NC(=O)N(C)c2cc(Nc3ccc(N4CCN(CC(=O)NCCOCCC(=O)O)CC4)cc3)ncn2)c1Cl]

### 3-(2-(2-(4-(4-((6-(3-(2,6-dichloro-3,5-dimethoxyphenyl)-1-methylureido)pyrimidin-4-yl)amino)phenyl)piperazin-1-yl)acetamido)ethoxy)propanoic acid

2-(4-(4-((6-(3-(2,6-dichloro-3,5-dimethoxyphenyl)-1-methylureido)pyrimidin-4-yl)amino)phenyl)piperazin-1-yl)acetic acid (11 mg, 0.025 mmol) and tert-butyl 3-(2-aminoethoxy)propanoate were dissolved in DMF (1 mL). DIEA was added (20 μL, 0.125 mmol) followed by HATU (19 mg, 0,050 mmol) and the reaction was stirred for 20 minutes, Water was added and the mixture was extracted with ethyl acetate. Combined organics were washed with brine, dried over Na₂SO₄, concentrated, and the crude residue was purified by silica gel chromatography to provide the tert-butyl ester. LC/MS found 760.7. This ester was then dissolved in DCM (1 mL) and TFA (1 mL) was added and the solution stirred at room temperature for 2 hours. The solvent was then removed under pressure to provide the title compound (9.9 mg, 0.014 mmol, 56%). LC/MS m/z calculated for [M+H]⁺ 705.22, found 704.80.

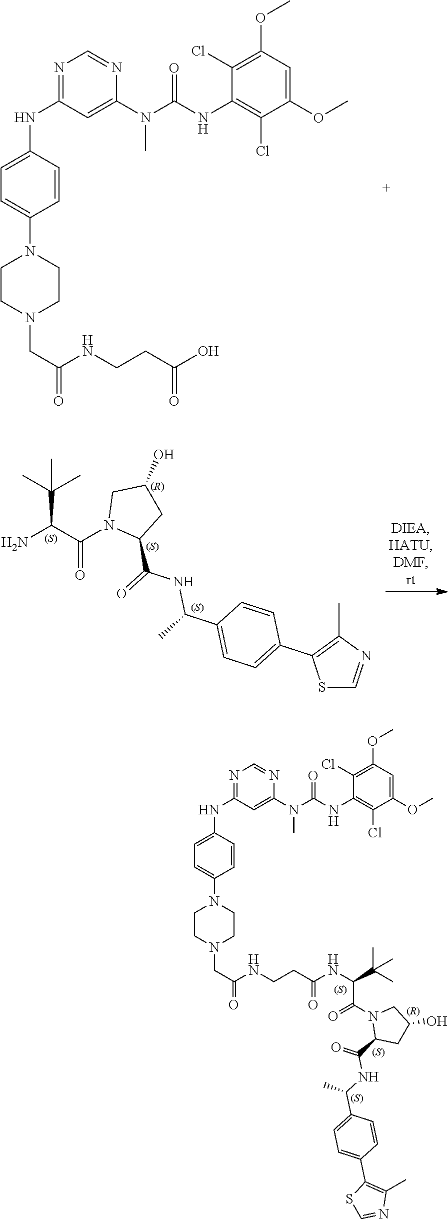

(3-(2-(2-(4-(4-((6-(3-(2,6-dichloro-3,5-dimethoxyphenyl)-1-methylureido)pyrimidin-4-yl)amino)phenyl)piperazin-1-yl)acetamido)ethoxy)propanoic acid (9.9 mg, 0.014 mmol) and (1R,4S)-2-((S)-2-amino-3,3-dimethylbutanoyl)-4-hydroxy-N-((R)-1-(4-(4-methylthiazol-5-yl)phenyl)ethyl)cyclopentane-1-carboxamide dihydrochloride (6.7 mg, 0.014 mmol) were dissolved in DMF (1 mL). DIEA (12 μL, 0.070 mmol) was added, followed by HATU (11 mg, 0.028 mmol). The reaction was stirred for 20 minutes before purification by HPLC to obtain bispecific compound 15 as a white solid and TFA salt (5.7 mg, 0.005 mmol, 36%). LC/MS m/z calculated for [M+H]⁺ 1131.43, found 1131.60. ¹H NMR (500 MHz, DMSO-d₆) δ 11.90 (s, 1H), 10.02 (s, 1H), 9.45 (s, 1H), 8.92 (s, 1H), 8.58 (s, 1H), 8.33 (s, 1H), 8.29 (d, J=8.2 Hz, 1H), 7.83 (d, J=9.3 Hz, 1H), 7.41 (d, J=8.7 Hz, 2H), 7.39-7.33 (m, 2H), 7.33-7.26 (m, 2H), 6.91 (d, J=9.1 Hz, 2H), 6.84 (s, 1H), 6.38 (d, J=1.0 Hz, 1H), 5.05 (s, 1H), 4.85 (p, J=7.3 Hz, 1H), 4.47 (d, J=9.4 Hz, 1H), 4.35 (s, 1H), 4.22 (s, 1H), 3.94 (s, 2H), 3.87 (s, 6H), 3.65 (s, 2H), 3.60-3.45 (m, 6H), 3.39 (d, J=5.7 Hz, 3H), 3.25 (s, 3H), 3.24 (s, 2H), 3.02 (s, 2H), 2.39 (s, 4H), 2.01-1.91 (m, 1H), 1.76-1.68 (m, 1H), 1.31 (d, J=7.0 Hz, 3H), 0.92 (s, 2H), 0.87 (s, 9H).

### Example 17: Synthesis of (2S,4R)-1-[(2S)-2-[3-(4-{4-[(6-{[(2,6-dichloro-3,5-dimethoxyphenyl)carbamoyl](methyl)amino}pyrimidin-4-yl)amino]phenyl}piperazin-1-yl)propanamido]-3,3-dimethylbutanoyl]-4-hydroxy-N-[(1R)-1-[4-(4-methyl-1,3-thiazol-5-yl)phenyl]ethyl]pyrrolidine-2-carboxamide (16)

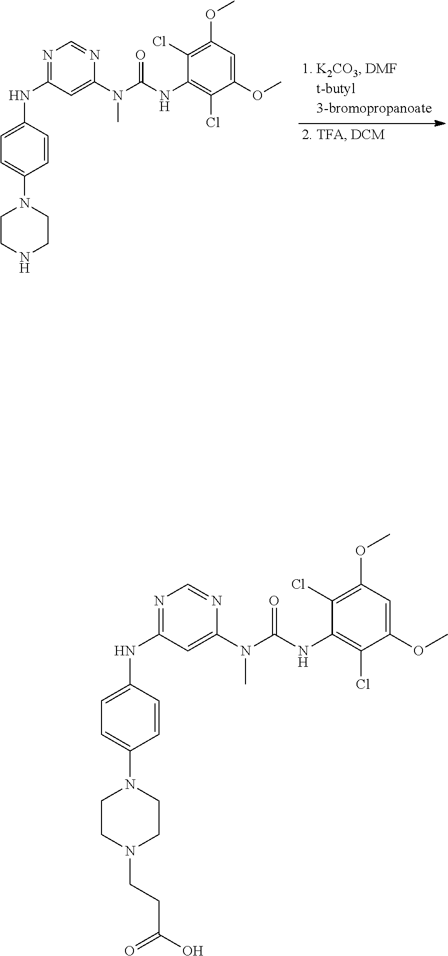 [SMILES: COc1cc(OC)c(Cl)c(NC(=O)N(C)c2cc(Nc3ccc(N4CCNCC4)cc3)ncn2)c1Cl ; COc1cc(OC)c(Cl)c(NC(=O)N(C)c2cc(Nc3ccc(N4CCN(CCC(=O)O)CC4)cc3)ncn2)c1Cl]

### 3-(4-(4-((6-(3-(2,6-dichloro-3,5-dimethoxyphenyl)-1-methylureido)pyrimidin-4-yl)amino)phenyl)piperazin-1-yl)propanoic acid

tert-Butyl 3-bromopropanoate (12 mg, 0.057 mmol) was added to a mixture of 3-(2,6-dichloro-3,5-dimethoxyphenyl)-1-methyl-1-(6-((4-(piperazin-1-yl)phenyl)amino)pyrimidin-4-yl)urea (20 mg, 0.038 mmol) and K₂CO₃ (16 mg, 0.11 mmol) in DMF (0.75 mL). The mixture was stirred at 50° C. overnight, then an additional 20 mg of bromide was added, and the mixture stirred at room temperature overnight. DCM and water were added, the mixture partitioned, the organics evaporated, and the crude residue purified by silica gel chromatography using a gradient from 0 to 15% MeOH (1.75N NH₃) in DCM to provide the tert-butyl ester. LC/MS found 659.90. The ester was then dissolved in DCM (1 mL) and TFA (1 mL) was added. After stirring at room temperature for 2 hours, the solvent was evaporated under pressure to provide the title compound (20 mg, 0.030, 79%). LC/MS m/z calculated for [M+H]⁺ 604.19, found 603.79.

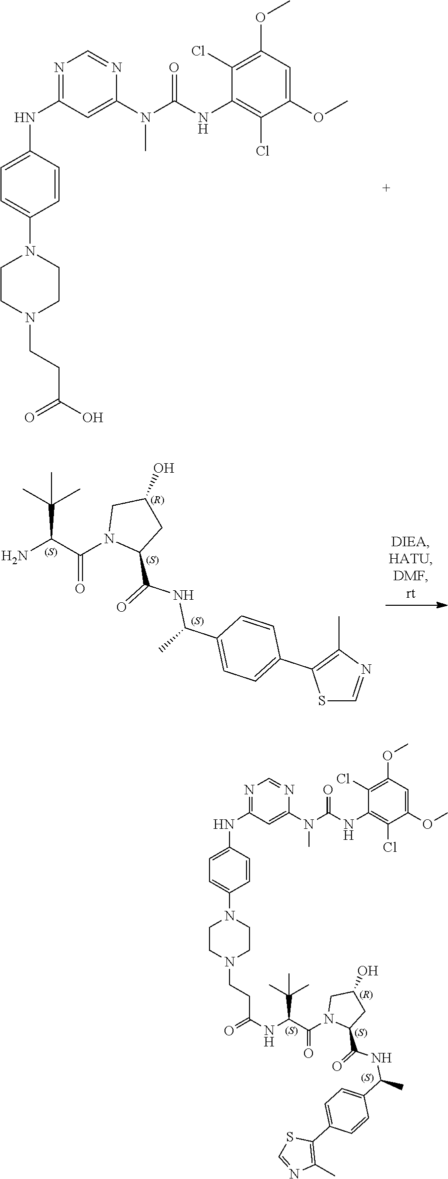

3-(4-(4-((6-(3-(2,6-dichloro-3,5-dimethoxyphenyl)-1-methylureido)pyrimidin-4-yl)amino)phenyl)piperazin-1-yl)propanoic acid (20 mg, 0.030 mmol) and (1R,4S)-2-((S)-2-amino-3,3-dimethylbutanoyl)-4-hydroxy-N-((R)-1-(4-(4-methylthiazol-5-yl)phenyl)ethyl)cyclopentane-1-carboxamide dihydrochloride (13 mg, 0.030 mmol) were dissolved in DMF (1 mL). DIEA (26 μL, 0.15 mmol) was added, shortly followed by HATU (23 mg, 0.060 mmol). After stirring for 20 minutes, DMSO was added and the mixture was purified by HPLC to provide the bispecific compound 16 as a white solid and TFA salt (15.2 mg, 0.015 mmol, 49%). LC/MS m/z calculated for [M+H]⁺ 1030.39, found 1029.64. ¹H NMR (500 MHz, DMSO-d₆) δ 11.90 (s, 1H), 9.53 (s, 1H), 9.46 (s, 1H), 8.92 (s, 1H), 8.40-8.26 (m, 2H), 8.20 (d, J=9.2 Hz, 1H), 7.51-7.34 (m, 4H), 7.31 (d, J=8.2 Hz, 2H), 6.39 (s, 1H), 6.93 (d, J=9.1 Hz, 2H), 6.84 (s, 1H), 4.88 (s, 1H), 4.90-4.79 (m, 1H), 4.48 (d, J=9.2 Hz, 1H), 4.36 (s, 1H), 4.23 (d, J=4.2 Hz, 2H), 3.87 (s, 6H), 3.76-3.66 (m, 2H), 3.60-3.41 (m, 4H), 3.34 (s, 2H), 3.25 (d, J=3.0 Hz, 3H), 3.12 (s, 2H), 2.90 (d, J=12.5 Hz, 2H), 2.80-2.70 (m, 1H), 2.69 (d, J=8.3 Hz, 1H), 2.04-1.85 (m, 1H), 1.79-1.71 (m, 1H), 1.31 (d, J=7.0 Hz, 3H), 0.90 (s, 9H).

### Example 18: Synthesis of (2S,4R)-1-[(2S)-2-[4-(4-{4-[(6-{[(2,6-dichloro-3,5-dimethoxyphenyl)carbamoyl](methyl)amino}pyrimidin-4-yl)amino]phenyl}piperazin-1-yl)butanamido]-3,3-dimethylbutanoyl]-4-hydroxy-N-[(1R)-1-[4(4-methyl-1,3-thiazol-5-yl)phenyl]ethyl]pyrrolidine-2-carboxamide (17)

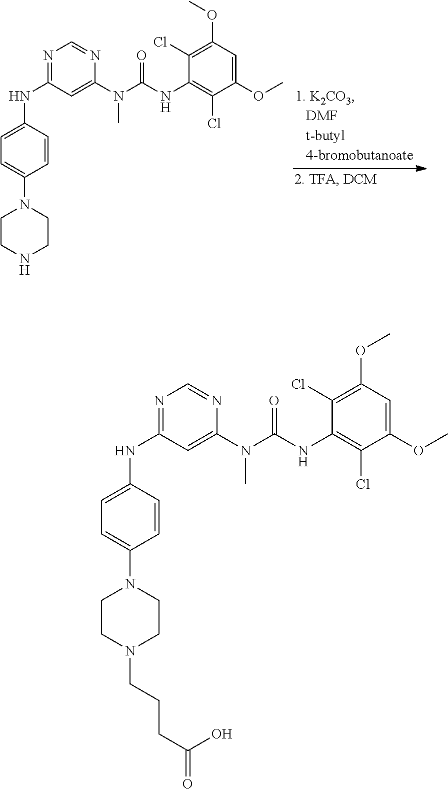 [SMILES: COc1cc(OC)c(Cl)c(NC(=O)N(C)c2cc(Nc3ccc(N4CCNCC4)cc3)ncn2)c1Cl ; COc1cc(OC)c(Cl)c(NC(=O)N(C)c2cc(Nc3ccc(N4CCN(CCCC(=O)O)CC4)cc3)ncn2)c1Cl]

### 4-(4-(4-((6-(3-(2,6-dichloro-3,5-dimethoxyphenyl)-1-methylureido)pyrimidin-4-yl)amino)phenyl)piperazin-1-yl)butanoic acid

tert-Butyl 4-bromobutanoate (13 mg, 0.057 mmol) was added to a mixture of 3-(2,6-dichloro-3,5-dimethoxyphenyl)-1-methyl-1-(6-((4-(piperazin-1-yl)phenyl)amino)pyrimidin-4-yl)urea (20 mg, 0.038 mmol) and K₂CO₃ (16 mg, 0.11 mmol) in DMF (0.75 mL). The mixture was stirred at 50° C. for 3 hours, an additional 9 mg of bromide was added, and the mixture stirred overnight. The reaction was diluted with water and DCM, partitioned, and the organic layer was evaporated. The crude residue was purified by silica gel chromatography using a gradient from 0 to 15% MeOH (1.75N NH₃) in DCM to provide the tert-butyl ester. LC/MS found 673.80. The residue was then dissolved in 1 mL DCM and 0.5 mL TFA was added. The solution was stirred for 1 hour and the solvent evaporated under pressure to obtain the title compound (17 mg, 0.023 mmol, 61%). LC/MS m/z calculated for [M+H]⁺ 618.19, found 617.99.

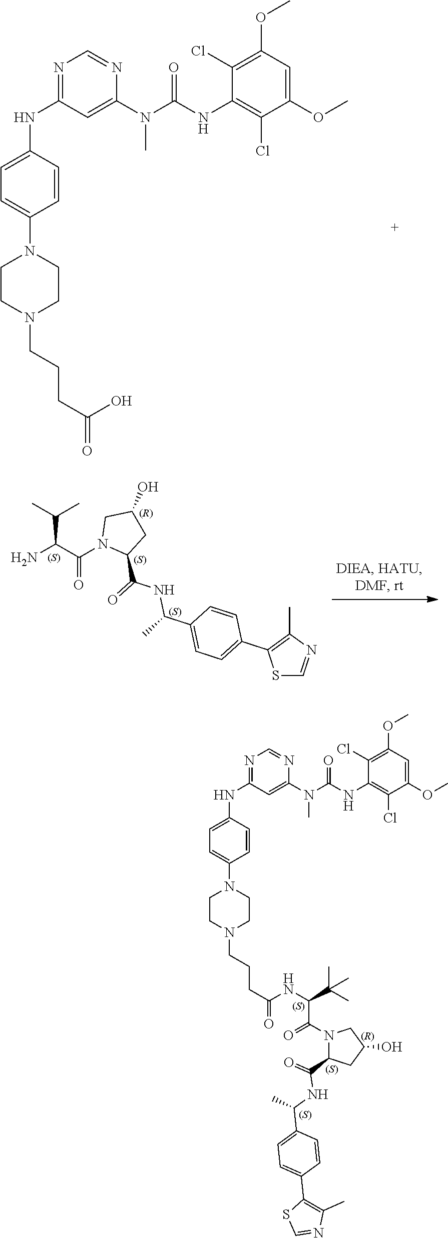

4-(4-(4-((6-(3-(2,6-dichloro-3,5-dimethoxyphenyl)-1-methylureido)pyrimidin-4-yl)amino)phenyl)piperazin-1-yl)butanoic acid (17 mg, 0.023 mmol) and (1R,4S)-2-((S)-2-amino-3,3-dimethylbutanoyl)-4-hydroxy-N-((R)-1-(4-(4-methylthiazol-5-yl)phenyl)ethyl)cyclopentane-1-carboxamide dihydrochloride (12 mg, 0,023 mmol) were dissolved in DMF (1 mL). DIEA (24 μL, 0.14 mmol) was added, followed by HATU (17 mg, 0.046 mmol). After stirring for 20 minutes, the solution was diluted with DMSO and purified by HPLC to provide bispecific compound 17 as a white solid and TFA salt (15.0 mg, 0.013 mmol, 56%). LC/MS m/z calculated for [M+H]⁺ 1044.40, found 1043.73. ¹H NMR (500 MHz, DMSO-d₆) δ 11.91 (s, 1H), 9.71 (s, 1H), 9.46 (s, 1H), 8.92 (s, 1H), 8.33 (s, 1H), 8.30 (d, J=7.8 Hz, 1H), 7.99 (d, J=9.2 Hz, 1H), 7.46-7.34 (m, 4H), 7.34-7.28 (m, 2H), 6.93 (d, J=8.8 Hz, 2H), 6.84 (s, 1H), 6.39 (s, 1H), 4.92-4.77 (m, 1H), 4.47 (d, J=9.3 Hz, 1H), 4.36 (t, J=8.1 Hz, 1H), 4.26-4.17 (m, 1H), 3.87 (s, 6H), 3.70 (d, J=12.7 Hz. 2H), 3.58-3.45 (m, 5H), 3.25 (s, 3H), 3.09 (d, J=11.0 Hz, 6H), 2.95-2.88 (m, 1H), 2.39 (s, 3H), 2.09-1.92 (m, 1H), 1.85 (s, 1H), 1.74 (m, 1H), 1.31 (d, J=7.0 Hz, 3H), 0.89 (s, 9H).

### Example 19: Synthesis of (2S,4R)-1-[(2S)-2-[6-(4-{4-[(6-{[(2,6-dichloro-3,5-dimethoxyphenyl)carbamoyl](methyl)amino}pyrimidin-4-yl)amino]phenyl}piperazin-1-yl)hexanamido]-3,3-dimethylbutanoyl]-4-hydroxy-N-[(1R)-1-[4-(4-methyl-1,3-thiazol-5-yl)phenyl]ethyl]pyrrolidine-2-carboxamide (18)

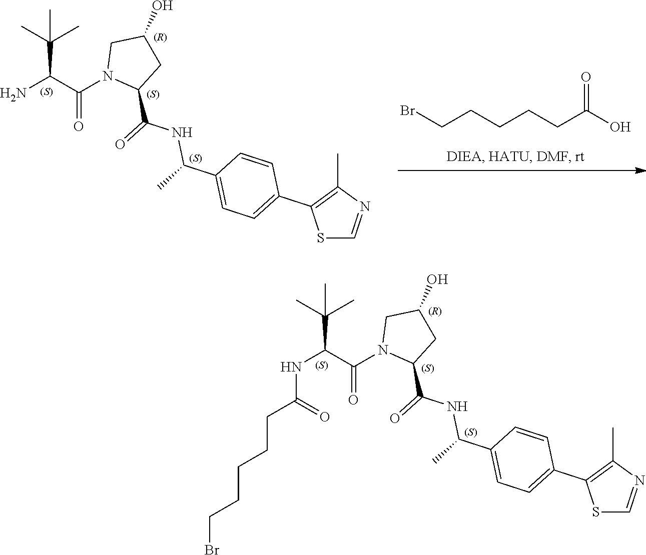

### (1R,4S)-2-((S)-2-(6-bromohexanamido)-3,3-dimethylbutanoyl)-4-hydroxy-N-((R)-1-(4-(4-methylthiazol-5-yl)phenyl)ethyl)cyclopentane-1-carboxamide

(1R,4S)-2-((S)-2-amino-3,3-dimethylbutanoyl)-4-hydroxy-N-((R)-1-(4-(4-methylthiazol-5-yl)phenyl)ethyl)cyclopentane-1-carboxamide dihydrochloride (15 mg, 0.029 mmol) and 6-bromohexanoic acid (8.5 mg, 0.044 mmol) were dissolved in DMF (1 mL). DIEA (25 μL, 0.015 mmol) was added followed by HATU (22 mg, 0.058 mmol). The reaction was stirred 15 minutes before it was diluted with water and DCM. The mixture was then partitioned, and the organic layer was concentrated and purified by silica gel chromatography to provide the title compound. LC/MS m/z calculated for [M+H]⁺ 620.21, found 620.89.

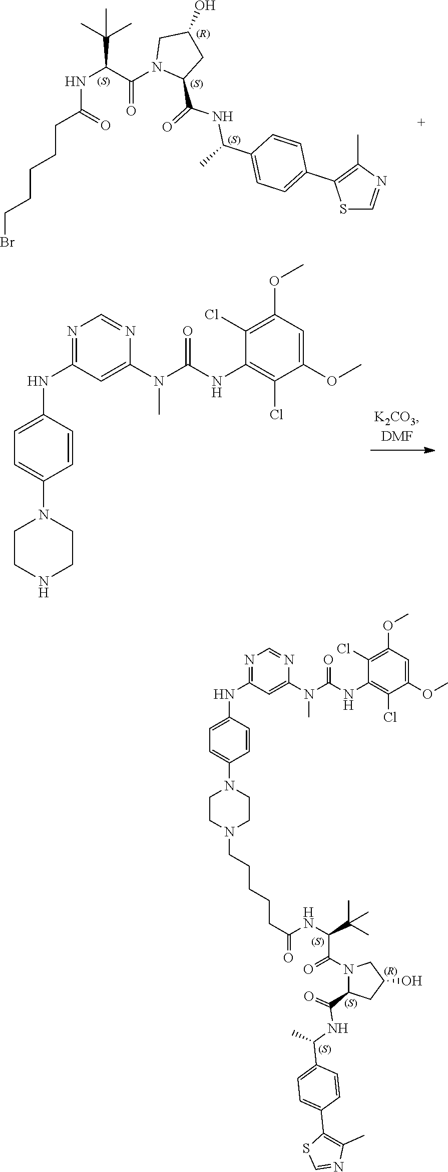

(1R,4S)-2-((S)-2-(6-bromohexanamido)-3,3-dimethylbutanoyl)-4-hydroxy-N-((R)-1-(4-(4-methylthiazol-5-yl)phenyl)ethyl)cyclopentane-1-carboxamide (18 mg, 0.029 mmol), K₂CO₃ (12 mg, 0.087 mmol), and 3-(2,6-dichloro-3,5-dimethoxyphenyl)-1-methyl-1-(6-((4-(piperazin-1-yl)phenyl)amino)pyrimidin-4-yl)urea (15 mg, 0.029 mmol) were dissolved in DMF (1 mL) and stirred at room temperature for 2 days. The temperature was then raised to 50° C. for 6 hours. The mixture was then filtered, diluted in DMSO and purified by HPLC to provide bispecific compound 18 as a white solid and a TFA salt (12.2 mg, 0.010 mmol, 34%). LC/MS m/z calculated for [M+H]⁺ 1072.43, found 1071.64. ¹H NMR (500 MHz, DMSO-d₆) δ 11.90 (s, 1H), 9.46 (s, 2H), 8.92 (s, 1H), 8.43-8.14 (m, 2H), 7.76 (d, J=9.4 Hz, 1H), 7.51-7.17 (m, 6H), 7.04-6.85 (m, 2H), 6.84 (s, 1H), 6.39 (d, J=1.1 Hz, 1H), 4.90-4.76 (m, 1H), 4.47 (d, J=9.4 Hz, 1H), 4.35 (t, J=8.1 Hz, 1H), 4.22 (d, J=3.3 Hz, 1H), 3.87 (s, 6H), 3.70 (d, J=12.9 Hz, 2H), 3.53 (t, J=11.0 Hz, 4H), 3.25 (s, 3H), 3.08 (d, J=11.0 Hz, 4H), 2.88 (s, 2H), 2.39 (s, 3H), 2.23 (dt, J=14.7, 7.6 Hz, 1H), 2.10 (d, J=7.2 Hz, 1H), 1.95 (td, J=9.4, 8.1, 4.6 Hz, 1H), 1.73 (m, 1H), 1.62 (q, J=14.3, 10.7 Hz, 2H), 1.52-1.38 (m, 3H), 1.31 (d, J=7.0 Hz, 3H), 1.27-1.12 (m, 2H), 0.87 (d, J=7.0 Hz, 9H).

### Example 20: Synthesis of (2S,4R)-1-[(2S)-2-(2-{4-[3-({7-[(tert-butylcarbamoyl)aminol-6-(3,5-dimethoxyphenyl)pyrido[2,3-d]pyrimidin-2-yl}amino)propyl]piperazin-1-yl}acetamido)-3,3-dimethylbutanoyl]-4-hydroxy-N-[(1R)-1-[4-(4-methyl-1,3-thiazol-5-yl)phenyl]ethyl]pyrrolidine-2-carboxamide (19)

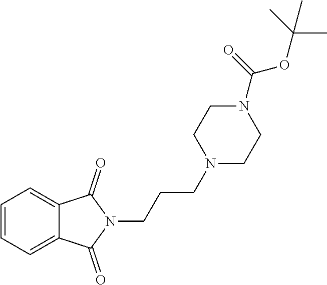 [SMILES: CC(C)(C)OC(=O)N1CCN(CCCN2C(=O)c3ccccc3C2=O)CC1]

### tert-Butyl 4-(3-(1,3-dioxoisoindolin-2-yl)propyl)piperazine-1-carboxylate

N-Boc piperazine (1.0 g, 5.37 mmol) and N-(3-bromopropyl)phthalimide (1.44 g, 5.37 mmol) were dissolved in DMF (15 mL). K₂CO₃ (1.48 g, 10.74 mmol) and NaI (1.37 g, 8.06 mmol) were added, and the mixture stirred at room temperature for 24 hours. DCM (80 mL) was added, the mixture filtered, the filtrate evaporated, and the residue purified by silica gel chromatography using a gradient from 0-10% MeOH (1.75 N NH₃) in DCM to provide the title compound as pale yellow crystals (1.71 g, 4.59 mmol, 85%). LC/MS m/z calculated for [M+H]⁺ 374.20, found 373.97.

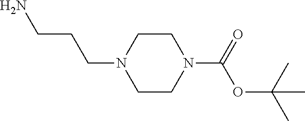 [SMILES: CC(C)(C)OC(=O)N1CCN(CCCN)CC1]

### tert-butyl 4-(3-aminopropyl)piperazine-1-carboxylate

tert-Butyl 4-(3-(1,3-dioxoisoindolin-2-yl)propyl)piperazine-1-carboxylate (819 mg, 2.20 mmol) was dissolved in EtOH (12 mL) and hydrazine hydrate (50%, 700 μL, 11.0 mmol) was added. A white precipitate formed as the reaction stirred at room temperature overnight. DCM (50 mL) was added and the mixture stirred for 30 min before it was filtered to yield a clear solution. The filtrate was concentrated to yield a white solid, which was dissolved in DCM and filtered again. The filtrate was again concentrated to provide the title compound as a pale yellow oil (543 mg, 2.22 mmol, 101%). LC/MS m/z calculated for [M+H]⁺ 244.19, found 244.09.

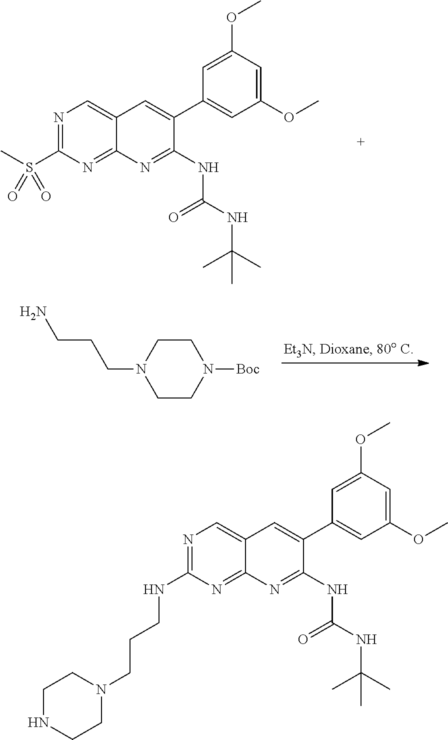

### 1-(tert-butyl)-3-(6-(3,5-dimethoxyphenyl)-2-((3-(piperazin-1-yl)propyl)amino)pyrido[2,3-d]pyrimidin-7-yl)urea

1-(tert-butyl)-3-(6-(3,5-dimethoxyphenyl)-2-(methylsulfonyl)pyrido[2,3-d]pyrimidin-7-yl)urea (459 mg, 1.0 mmol) was dissolved in dioxane (5 mL). Et₃N (178 μL, 1.0 mmol) was added, followed by tert-butyl 4-(3-aminopropyl)piperazine-1-carboxylate (267 mg, 1.1 mmol). The solution was stirred at 50° C. for 6 hours. The solvent was evaporated and the residue purified by silica gel chromatography using a gradient of 0-10% MeOH (1.75N NH₃) in DCM to provide tert-butyl 4-(3-((7-(3-(tert-butyl)ureido)-6-(3,5-dimethoxyphenyl)pyrido[2,3-d]pyrimidin-2-yl)amino)propyl)piperazine-1-carboxylate (594 mg, 0.96 mmol, 96%). The carbamate was then dissolved in DCM (2 mL) and TFA (1 mL) was added. The reaction was stirred for 2 hours and the solvent was removed under pressure. The residue was then dissolved in THF and saturated aqueous NaHCO₃ was added and the suspension stirred for 2 h before being filtered. The solid was washed with water, allowed to dry, before being dissolved in 8 mL DCM and 200 μL 4M HCl in dioxane, filtered and concentrated to provide the title compound as an HCl salt (439 mg, 0.79 mmol, 82%). LC/MS m/z calculated for [M+H]⁺ 523,31, found 523,08.

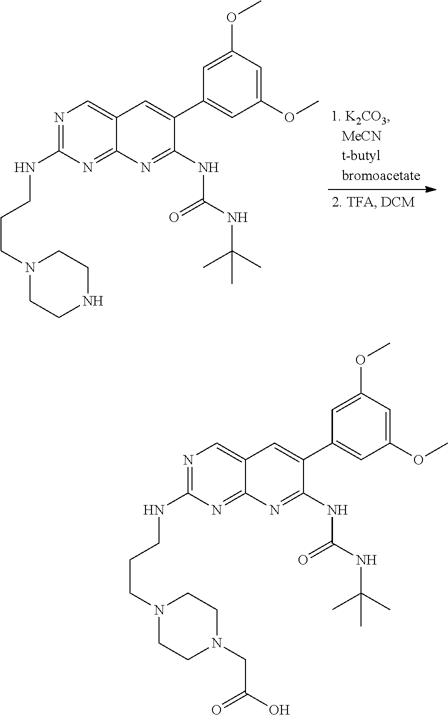 [SMILES: COc1cc(OC)cc(-c2cc3cnc(NCCCN4CCNCC4)nc3nc2NC(=O)NC(C)(C)C)c1 ; COc1cc(OC)cc(-c2cc3cnc(NCCCN4CCN(CC(=O)O)CC4)nc3nc2NC(=O)NC(C)(C)C)c1]

### 2-(4-(3-((7-(3-(tert-butyl)ureido)-6-(3,5-dimethoxyphenyl)pyrido[2,3-d]pyrimidin-2-yl)amino)propyl)piperazin-1-yl)acetic acid

1-(tert-butyl)-3-(6-(3,5-dimethoxyphenyl)-2-((3-(piperazin-1-yl)propyl)amino)pyrido[2,3-d]pyrimidin-7-yl)urea HCl (17 mg, 0.030 mmol) and K₂CO₃ (17 mg, 0.12 mmol) were added to MeCN (1 mL). tert-Butyl bromoacetate (8.8 mg, 0.045 mmol) was then added and the reaction stirred at room temperature overnight. The solvent was evaporated and the residue purified by silica gel chromatography using a gradient of 0-10% MeOH (1.75N NH₃) in DCM to provide tert-butyl 2-(4-(3-((7-(3-(tert-butyl)ureido)-6-(3,5-dimethoxyphenyl)pyrido[2,3-d]pyrimidin-2-yl)amino)propyl)piperazin-1-yl)acetate. LC/MS m/z calculated for [M+2H-tBu]+290.65, found 291.1. DCM (1 mL) and TFA (1 mL) were added, and the solution stirred at 2 hours at room temperature. The solvent was removed to obtain the title compound as a TFA salt (8.9 mg, 0.017 mmol, 57%). LC/MS m/z calculated for [M+H]⁺ 581.31. found 580.89.

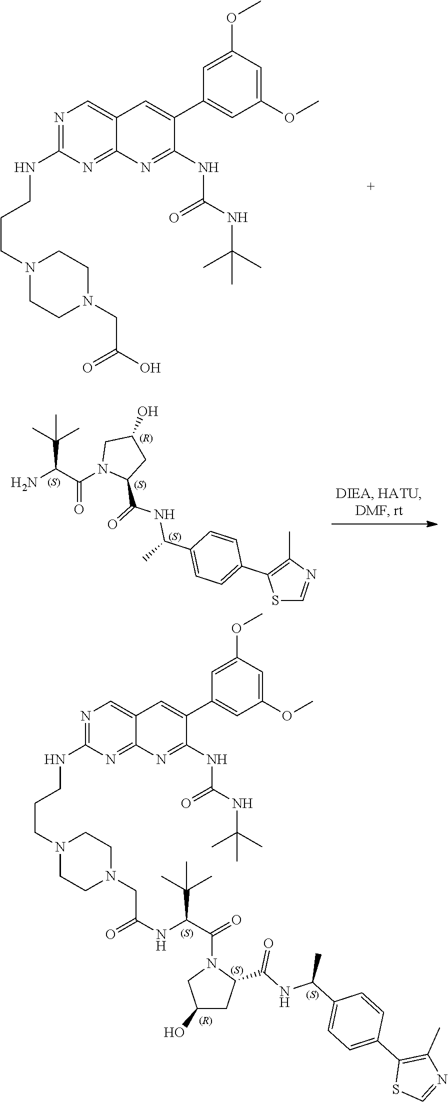

2-(4-(3-((7-(3-(tert-butyl)ureido)-6-(3,5-dimethoxyphenyl)pyrido[2,3-d]pyrimidin-2-yl)amino)propyl)piperazin-1-yl)acetic acid (8.9 mg, 0.017 mmol) and (1R,4S)-2-((S)-2-amino-3,3-dimethylbutanoyl)-4-hydroxy-N-((R)-1-(4-(4-methylthiazol-5-yl)phenyl)ethyl)cyclopentane-1-carboxamide dihydrochloride (7.5 mg, 0.017 mmol) were dissolved in DMF (1 mL), DIEA (15 μL, 0.085 mmol) was added, followed by HATU (13 mg, 0.034 mmol). The reaction was stirred for 20 minutes before purification by HPLC to obtain bispecific compound 19 as a yellow solid and a TFA salt (16.8 mg, 0.015 mmol, 88%). LC/MS m/z calculated for [M+2H]²+504.26, found 504.20.

### Example 21. Synthesis of (2S,4R)-1-[(2S)-2-(4-{4-[3-({7-[(tert-butylcarbamoyl)aminol-6-(3,5-dimethoxyphenyl)pyrido[2,3-d]pyrimidin-2-yl}amino)propyl]piperazin-1-yl}butanamido)-3,3-dimethylbutanoyl]-4-hydroxy-N-[(1R)-1-[4-(4-methyl-1,3-thiazol-5-yl)phenyl]ethyl]pyrrolidine-2-carboxamide (20)

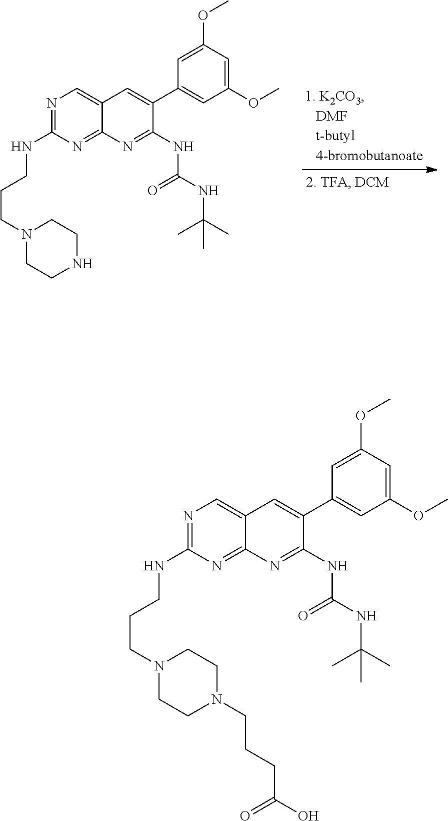 [SMILES: COc1cc(OC)cc(-c2cc3cnc(NCCCN4CCNCC4)nc3nc2NC(=O)NC(C)(C)C)c1 ; COc1cc(OC)cc(-c2cc3cnc(NCCCN4CCN(CCCC(=O)O)CC4)nc3nc2NC(=O)NC(C)(C)C)c1]

### 4-(4-(3-((7-(3-(tert-butyl)ureido)-6-(3,5-dimethoxyphenyl)pyrido[2,3-d]pyrimidin-2-yl)amino)propyl)piperazin-1-yl)butanoic acid

tert-Butyl 4-bromobutanoate (13 mg, 0.057 mmol) was added to a mixture of 1-(tert-butyl)-3-(6-(3,5-dimethoxyphenyl)-2-((3-(piperazin-1-yl)propyl)amino)pyrido[2,3-d]pyrimidin-7-yl)urea hydrochloride (20 mg, 0.038 mmol) and K₂CO₃ (16 mg, 0.11 mmol) in DMF (0.75 mL). The mixture was stirred at 50° C. for 3 hours, at which point additional bromide (13 mg) was added and the solution was stirred at room temperature overnight. Water and DCM were added, the mixture partitioned, and the organic layer was concentrated and purified by silica gel chromatography using a gradient of 0-10% MeOH (1.75N NH₃) in DCM to provide tert-butyl 4-(4-(3-((7-(3-(tert-butyl)ureido)-6-(3,5-dimethoxyphenyl)pyrido[2,3-d]pyrimidin-2-yl)amino)propyl)piperazin-1-yl)butanoate. LC/MS m/z calculated for [M+2H]+333.21, found 332.97. DCM (1 mL) and TFA (0.5 mL) were added, and the solution stirred at 2 hours at room temperature. The solvent was removed to obtain the title compound as a TFA salt (17 mg, 0.023 mmol, 61%). LC/MS m/z calculated for [M+H]⁺ 609.34, found 608.89.

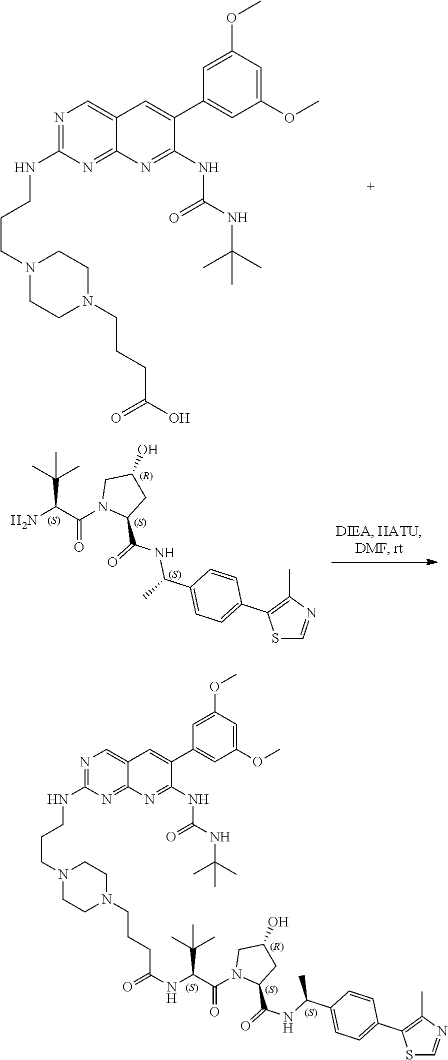

4-(4-(3-((7-(3-(tert-butyl)ureido)-6-(3,5-dimethoxyphenyl)pyrido[2,3-d]pyrimidin-2-yl)amino)propyl)piperazin-1-yl)butanoic acid (17 mg, 0.023 mmol) and (1R,4S)-2-((S)-2-amino-3,3-dimethylbutanoyl)-4-hydroxy-N-((R)-1-(4-(4-methylthiazol-5-yl)phenyl)ethyl)cyclopentane-1-carboxamide dihydrochloride (12 mg, 0.023 mmol) were dissolved in DMF (1 mL). DIEA (24 μL, 0.14 mmol) was added followed by HATU (17 mg, 0.046 mmol). The reaction was stirred for 20 minutes and purified by HPLC to obtain bispecific compound 20 as a yellow solid and a TFA salt (15 mg, 0.013 mmol, 56%). LC/MS m/z calculated for [M+H]⁺ 1035.55, found 1034.74.

### Example 22: Synthesis of (2S,4R)-1-[(2S)-2-{2-[4-(4-{[6-(3,5-dimethoxyphenyl)-7-oxo-8-[(4-propanamidophenyl)methyl]-5H,6H,7H,8H-[1,3]diazino[4,5-d]pyrimidin-2-yl]amino}phenyl)piperazin-1-yl]acetamido}-3,3-dimethylbutanoyl]-4-hydroxy-N-[(1R)-1-[4-(4-methyl-1,3-thiazol-5-yl)phenyl]ethyl]pyrrolidine-2-carboxamide (21)

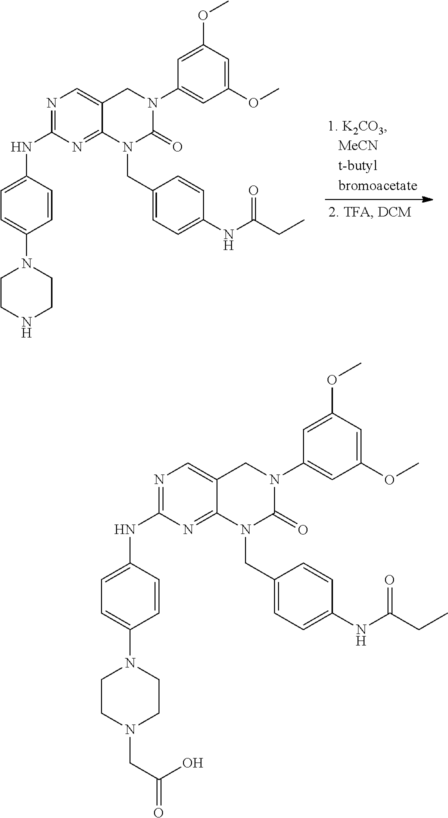 [SMILES: CCC(=O)Nc1ccc(CN2C(=O)N(c3cc(OC)cc(OC)c3)Cc3cnc(Nc4ccc(N5CCNCC5)cc4)nc32)cc1 ; CCC(=O)Nc1ccc(CN2C(=O)N(c3cc(OC)cc(OC)c3)Cc3cnc(Nc4ccc(N5CCN(CC(=O)O)CC5)cc4)nc32)cc1]

### 2-(4-(4-((6-(3,5-dimethoxyphenyl)-7-oxo-8-(4-propionamidobenzyl)-5,6,7,8-tetrahydropyrimido[4,5-d]pyrimidin-2-yl)amino)phenyl)piperazin-1-yl)acetic acid

N-(4-((3-(3,5-dimethoxyphenyl)-2-oxo-7-((4-(piperazin-1-yl)phenyl)amino)-3,4-dihydropyrimido[4,5-d]pyrimidin-1(2H)-yl)methyl)phenyl)propionamide (22 mg, 0.030 mmol) and K₂CO₃ (17 mg, 0.12 mmol) were added to MeCN (1 mL). tert-Butyl bromoacetate (9 mg) was then added, and the reaction stirred at room temperature overnight. The solvent was removed and the residue purified by silica gel chromatography using a gradient of 0-10% MeOH (1.75N NH₃) in DCM to provide tert-butyl 2-(4-(4-((6-(3,5-dimethoxyphenyl)-7-oxo-8-(4-propionamidobenzyl)-5,6,7,8-tetrahydropyrimido[4,5-d]pyrimidin-2-yl)amino)phenyl)piperazin-1-yl)acetate (8.2 mg, 0.011 mmol, 37%). LC/MS m/z calculated for [M+2H-tBu]²⁺ 340.69, found 340.89. The carbamate was then dissolved in DCM (1 mL) and TFA (1 mL) and stirred for 2 h at room temperature before the solvent was removed under pressure to provide the title compound. LC/MS m/z calculated for [M+H]⁺ 681.31, found 680.90.

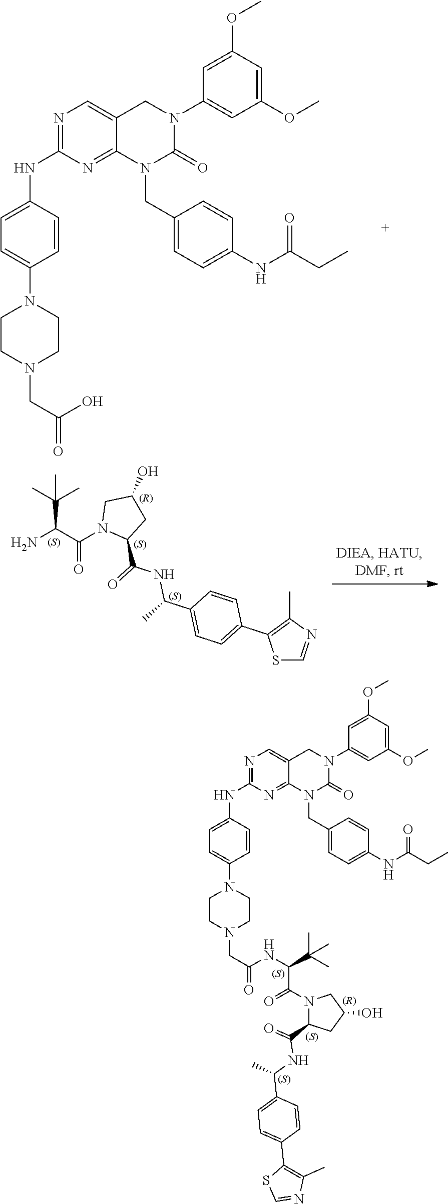

2-(4-(4-((6-(3,5-dimethoxyphenyl)-7-oxo-8-(4-propionamidobenzyl)-5,6,7,8-tetrahydropyrimido[4,5-d]pyrimidin-2-yl)amino)phenyl)piperazin-1-yl)acetic acid (8.1 mg, 0.11 mmol) and (1R,4S)-2-((S)-2-amino-3,3-dimethylbutanoyl)-4-hydroxy-N-((R)-1-(4-(4-methylthiazol-5-yl)phenyl)ethyl)cyclopentane-1-carboxamide dihydrochloride (5.5 mg, 0.011 mmol) were dissolved in DMF (1 mL). DIEA (10 μL, 0.055 mmol) was added, followed by HATU (8.4 mg, 0.022 mmol). The mixture was stirred for 20 minutes, then purified by HPLC to obtain bispecific compound 21 as a pale yellow solid and a TFA salt (6.8 mg, 0.0056 mmol, 51%). LC/MS m/z calculated for [M+2H]²+554.26, found 553.89. ¹H NMR (500 MHz, DMSO-d₆) δ 10.00 (s, 1H), 9.75 (s, 1H), 9.33 (s, 1H), 8.91 (s, 1H), 8.80-8.62 (m, 1H), 8.33 (d, J=7.8 Hz, 1H), 8.06 (s, 1H), 7.45 (d, J=8.6 Hz, 1H), 7.41-7.34 (m, 4H), 7.32 (d, J=8.4 Hz, 2H), 7.18 (d, J=8.2 Hz, 2H), 6.79 (d, J=9.0 Hz, 2H), 6.52 (d, J=2.2 Hz, 2H), 6.38 (t, J=2.2 Hz, 1H), 5.08 (s, 2H), 4.85 (q, J=6.9 Hz, 1H), 4.68 (s, 2H), 4.51 (d, J=9.1 Hz, 1H), 4.43-4.32 (m, 1H), 4.25 (s, 1H), 4.19-3.89 (m, 2H), 3.68 (s, 6H), 3.65-3.54 (m, 3H), 3.54-3.39 (m, 3H), 3.24 (s, 2H), 2.97 (s, 2H), 2.38 (s, 3H), 2.26-2.14 (m, 2H), 2.01-1.90 (m, 1H), 1.84-1.66 (m, 1H), 1.42 (d, J=7.0 Hz, 1H), 1.32 (d, J=7.0 Hz, 3H), 1.23-1.14 (m, 1H), 0.98 (t, J=7.5 Hz, 3H), 0.91 (s, 9H).

### Example 23: Synthesis of (2S,4R)-1-[(2S)-2-{4-[4-(4-{[6-(3,5-dimethoxyphenyl)-7-oxo-8-[(4-propanamidophenyl)methyl]-5H,6H,7H,8H-[1,3]diazino[4,5-d]pyrimidin-2-yl]amino]phenyl)piperazin-1-yl]butanamido}-3,3-dimethylbutanoyl]-4-hydroxy-N-[(1R)-1-[4-(4-methyl-1,3-thiazol-5-yl)phenyl]ethyl]pyrrolidine-2-carboxamide (22)

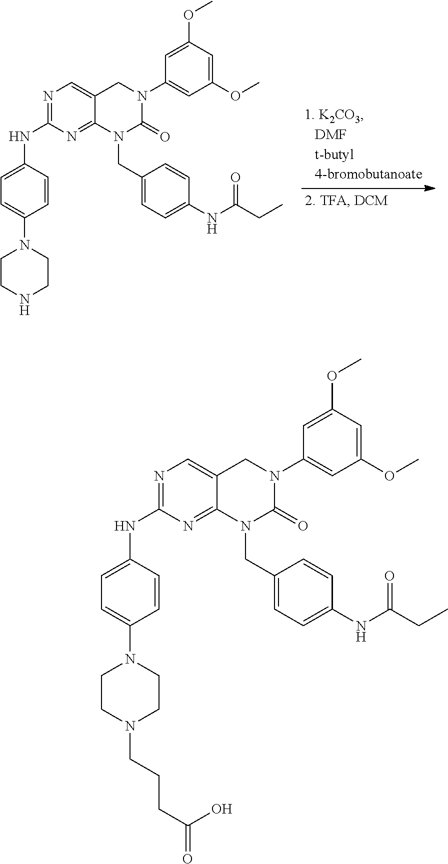 [SMILES: CCC(=O)Nc1ccc(CN2C(=O)N(c3cc(OC)cc(OC)c3)Cc3cnc(Nc4ccc(N5CCNCC5)cc4)nc32)cc1 ; CCC(=O)Nc1ccc(CN2C(=O)N(c3cc(OC)cc(OC)c3)Cc3cnc(Nc4ccc(N5CCN(CCCC(=O)O)CC5)cc4)nc32)cc1]

### 4-(4-(4-((6-(3,5-dimethoxyphenyl)-7-oxo-8-(4-propionamidobenzyl)-5,6,7,8-tetrahydropyrimido[4,5-d]pyrimidin-2-yl)amino)phenyl)piperazin-1-yl)butanoic acid

tert-Butyl 4-bromobutanoate (9 mg, (0.041 mmol) was added to a mixture of N-(4-((3-(3,5-dimethoxyphenyl)-2-oxo-7-((4-(piperazin-1-yl)phenyl)amino)-3,4-dihydropyrimido[4,5-d]pyrimidin-1(2H)-yl)methyl)phenyl)propionamide (20 mg, 0.027 mmol) and K₂CO₃ (15 mg, 0.11 mmol) in DMF (1 mL). The reaction was stirred at 50° C. for 3 hours, at which point additional bromide (9 mg) was added and the reaction stirred at room temperature overnight. Water and DCM were added, the mixture partitioned, organics concentrated and purified by silica gel chromatography to provide tert-butyl 4-(4-(4-((6-(3,5-dimethoxyphenyl)-7-oxo-8-(4-propionamidobenzyl)-5,6,7,8-tetrahydropyrimido[4,5-d]pyrimidin-2-yl)amino)phenyl)piperazin-1-yl)butanoate. The ester was then dissolved in DCM (1 mL) and TFA (1 mL) and stirred for 2 hours before the solvent was removed under pressure to afford the title compound (19 mg, 0.023 mmol, 85%). LC/MS m/z calculated for [M+H]⁺ 709.34, found 709.00.

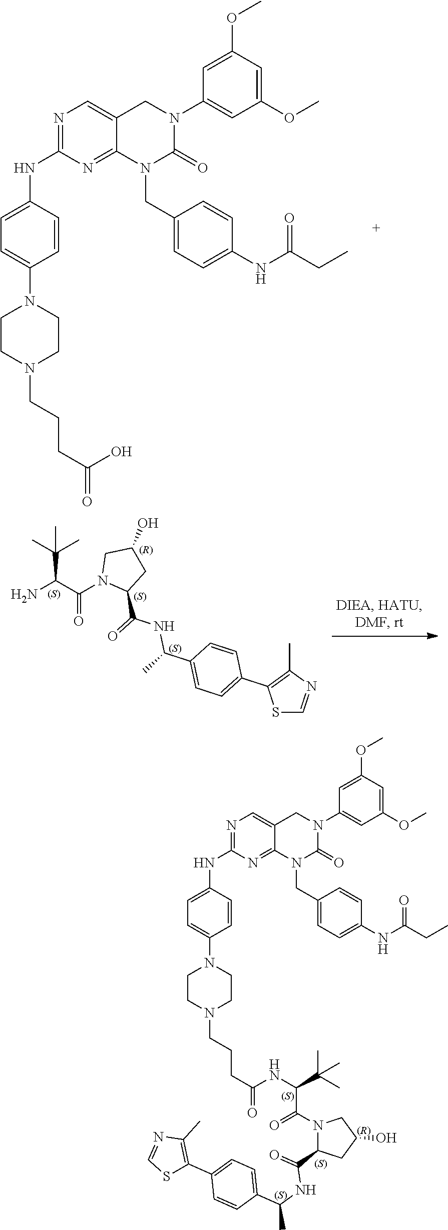

4-(4-(4-((6-(3,5-dimethoxyphenyl)-7-oxo-8-(4-propionamidobenzyl)-5,6,7,8-tetrahydropyrimido[4,5-d]pyrimidin-2-yl)amino)phenyl)piperazin-1-yl)butanoic acid (19 mg, 0.023 mmol) and (1R,4S)-2-((S)-2-amino-3,3-dimethylbutanoyl)-4-hydroxy-N-((R)-1-(4-(4-methylthiazol-5-yl)phenyl)ethyl)cyclopentane-1-carboxamide dihydrochloride (12 mg, 0.023 mmol) were dissolved in DMF (1 mL). DIEA (40 μL, 0.23 mmol) was added followed by HATU (17 mg, 0.046 mmol). The mixture was stirred for 20 minutes, then purified by HPLC to obtain bispecific compound 22 as a TFA salt and yellow solid (9.4 mg, 0.0075 mmol, 33%). LC/MS m/z calculated for [M+H]⁺ 1135.55, found 1134.92. ¹H NMR (500 MHz, DMSO-d₆) δ 9.75 (s, 1H), 9.50 (s, 1H), 9.31 (s, 1H), 8.92 (s, 1H), 8.30 (d, J=7.8 Hz, 1H), 8.06 (d, J=3.1 Hz, 1H), 7.99 (d, J=9.3 Hz, 1H), 7.51-7.41 (m, 1H), 7.41-7.34 (m, 4H), 7.34-7.27 (m, 2H), 7.18 (d, J=8.4 Hz, 2H), 6.82 (d, J=9.1 Hz, 1H), 6.52 (d, J=2.3 Hz, 2H), 6.38 (t, J=2.2 Hz, 1H), 5.08 (s, 2H), 4.90-4.77 (m, 1H), 4.68 (s, 2H), 4.48 (d, J=9.3 Hz, 1H), 4.36 (t, J=8.1 Hz, 1H), 4.23 (s, 1H), 3.68 (s, 6H), 3.65 (s, 2H), 3.57-3.46 (m, 6H), 3.09 (d, J=11.5 Hz, 6H), 2.84 (t, J=12.0 Hz, 2H), 2.39 (s, 2H), 2.21 (d, J=7.6 Hz, 2H), 1.95 (d, J=9.9 Hz, 1H), 1.84 (d, J=7.9 Hz, 3H), 1.74 (s, 1H), 1.42 (d, J=7.0 Hz, 1H), 1.31 (d, J=7.0 Hz, 3H), 0.99 (t, J=7.5 Hz, 3H), 0.88 (s, 9H).

### Example 24: Synthesis of (2S,4R)-1-[(2S)-2-{4-[(3S)-3-{4-amino-3-[2-(3,5-dimethoxyphenyl)ethynyl]-1H-pyrazolo[3,4-d]pyrimidin-1-yl}pyrrolidin-1-yl]butanamido}-3,3-dimethylbutanoyl]-4-hydroxy-N-[(1R)-1-[4-(4-methyl-1,3-thiazol-5-yl)phenyl]ethyl]pyrrolidine-2-carboxamide (23)

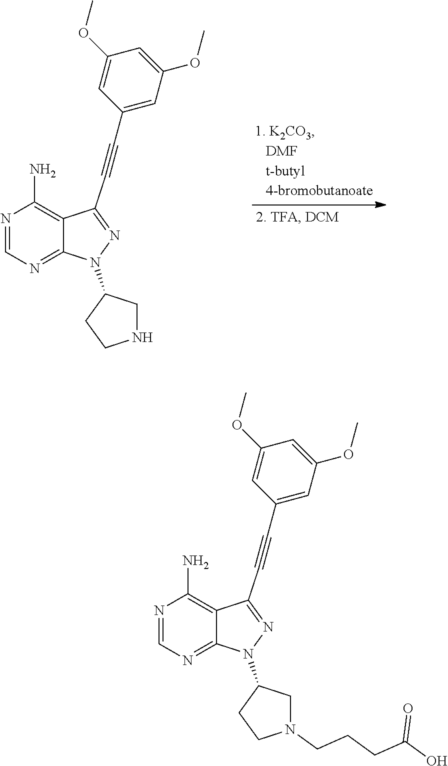 [SMILES: COc1cc(C#Cc2nn([C@H]3CCNC3)c3ncnc(N)c23)cc(OC)c1 ; COc1cc(C#Cc2nn([C@H]3CCN(CCCC(=O)O)C3)c3ncnc(N)c23)cc(OC)c1]

### (S)-4-(3-(4-amino-3-((3,5-dimethoxyphenyl)ethynyl)-1H-pyrazolo[3,4-d]pyrimidin-1-yl)pyrrolidin-1-yl)butanoic acid

(S)-3-((3,5-dimethoxyphenyl)ethynyl)-1-(pyrrolidin-3-yl)-1H-pyrazolo[3,4-d]pyrimidin-4-amine TFA (15 mg, 0.031 mmol, prepared similarly to as described in WO2013108809A1) and K₂CO₃ (12.8 mg, 0.093) were added to DMF (1 mL), followed by tert-butyl 4-bromobutanoate (11 mg, 0.047 mmol). The mixture was stirred for 3 hours at 50° C., additional bromide (11 mg) was added, and then stirred at room temperature overnight. The solvent was evaporated, and the residue purified by silica gel chromatography to provide the tert-butyl ester. LC/MS m/z calculated for [M+H]⁺ 507.26, found 506.97. The ester was then dissolved in DCM (1 mL) and TFA (1 mL) and stirred for 2 hours before the solvent was removed to afford the title compound (10 mg, 0.016 mmol, 52%). LC/MS m/z calculated for [M+H]⁺ 451.20. found 450.98.

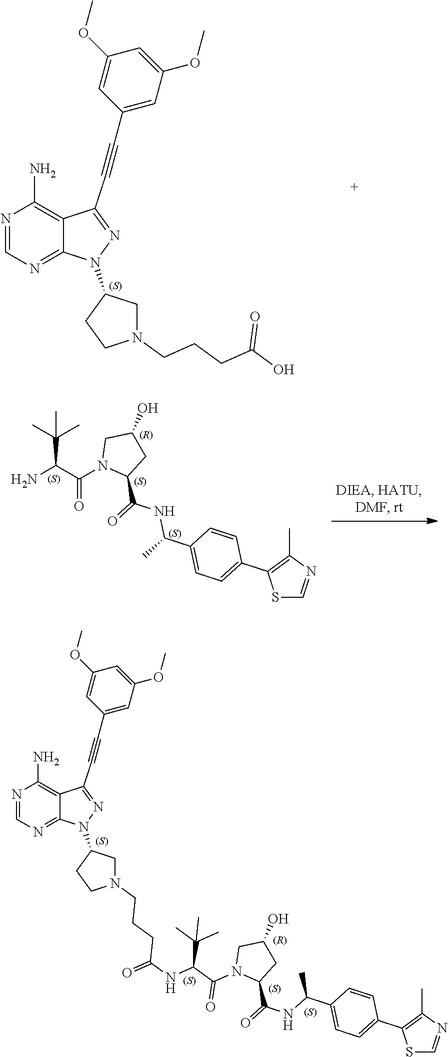

(S)-4-(3-(4-amino-3-((3,5-dimethoxyphenyl)ethynyl)-1H-pyrazolo[3,4-d]pyrimidin-1l-yl)pyrrolidin-1-yl)butanoic acid TFA (10 mg, 0.016 mmol) and (1R,4S)-2-((S)-2-amino-3,3-dimethylbutanoyl)-4-hydroxy-N-((R)-1-(4-(4-methylthiazol-5-yl)phenyl)ethyl)cyclopentane-1-carboxamide dihydrochloride (8.3 mg, 0.016 mmol) were dissolved in DMF (1 mL). DIEA (28 μL, 0.16 mmol) was added, followed by HATU (12 mg, 0.032). The mixture was stirred for 20 minutes, then purified by HPLC to obtain bispecific compound 23 as a TFA salt and white solid (11.2, 0.012 mmol, 73%). LC/MS m/z calculated for [M+H]⁺ 887.41, found 876.82. ¹H NMR (500 MHz, DMSO-d₆) δ 10.13 (s, 1H), 8.92 (s, 1H), 8.29 (d, J=7.8 Hz, 1H), 8.24 (s, 1H), 7.96 (t, J=9.5 Hz, 1H), 7.37 (d, J=8.2 Hz, 2H), 7.31 (d, J=8.1 Hz, 2H), 6.86-6.79 (m, 2H), 6.57 (t, J=2.3 Hz, 1H), 5.70-5.47 (m, 1H), 4.99 (s, 2H), 4.85 (s, 1H), 4.51-4.42 (m, 1H), 4.35 (t, J=8.1 Hz, 1H), 4.21 (s, 2H), 4.10 (s, 1H), 3.99-3.89 (m, 1H), 3.81 (s, 1H), 3.72 (s, 6H), 3.58-3.46 (m, 2H), 3.38 (d, J=11.3 Hz, 1H), 3.31-3.20 (m, 2H), 3.19-3.08 (m, 2H), 2.70-2.53 (m, 1H), 2.38 (s, 3H), 2.27-2.14 (m, 1H), 1.95 (t, J=10.4 Hz, 1H), 1.88-1.76 (m, 2H), 1.76-1.68 (m, 1H), 1.30 (d, J=7.0 Hz, 3H), 0.87 (s, 9H).

### Example 25: Knockdown of FGFR2 in Various Cell Lines

Kato III cells were treated with 0, 0.1, 1, or 10 M of bispecific compound 6 or 0.5 M THAL-SNS-032 (a known CDK9 degrader and positive control for CDK9 degradation; available from, e.g., MedChemExpress®, Cat. No. HY-10008, Monmouth Junction, N.J.) for 16 hours. Cells were then lysed in RIPA buffer (Millipore Sigma®) containing protease/phosphatase inhibitor cocktail (Roche®). The protein concentrations were measured by bicinchoninic acid assay (BCA) analysis (Pierce™). Equal amounts of protein were resolved by 4-12% Tris-Base gels (Invitrogen™), and then transferred to the Immuno-Blot PVDF membrane (Bio-Rad®), and immunoblotted with primary antibodies against FGFR2 (cell signaling), CDK9 (cell signaling) and f-Actin (cell signaling), and then immunoblotted with IRDye®800-labeled goat anti-rabbit IgG and IRDye®800-labeled goat anti-mouse IgG (LI-COR®) secondary antibodies. The membranes were detected on Odyssey CLx system.

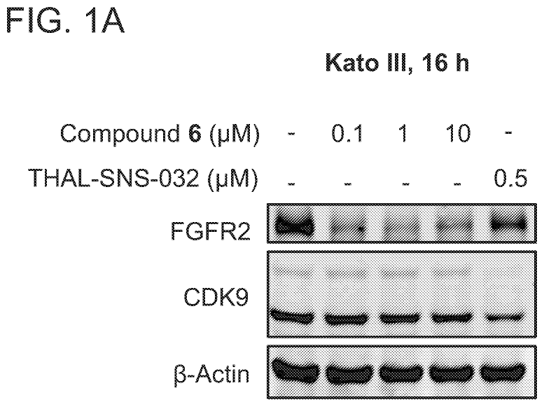

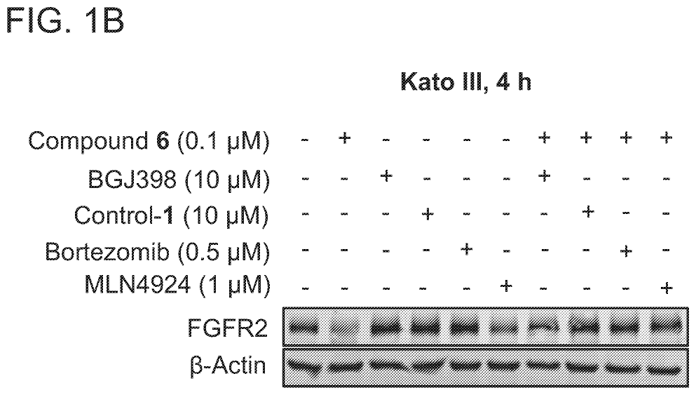

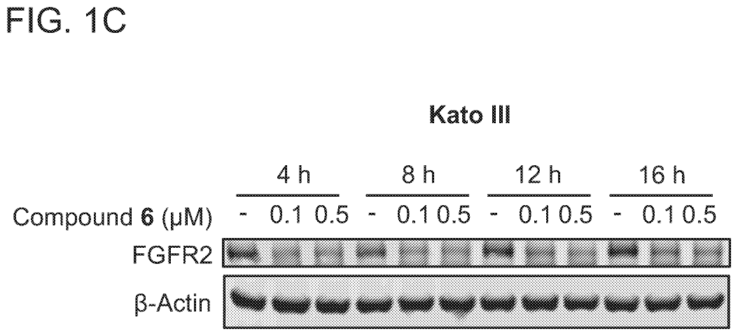

The results, illustrated in FIG. 1A, indicate that bispecific compound 6 induced the degradation of FGFR2 after 16 hours at the indicated concentrations and THAL-SNS-032 induced CDK9 degradation.

Kato III cells were pretreated with 10 μM BGJ398 (the parental compound and known FGFR1/2/3 inhibitor; available from, e.g., MedChemExpress®, Cat. No. HY-13311, Monmouth Junction, N.J.), 10 μM control-1 (VHL ligand), 0.5 M Bortezomib (a proteasome inhibitor; available from, e.g., Millipore Sigma®, Cat. No. 179324-69-7, Burlington, Mass.), and 1 μM MLN4924 (a neddylation inhibitor; available from, e.g., MedChemExpress®, Cat. No. HY-70062, Monmouth Junction, N.J.), for 2 h, and then treated with 0.1 M bispecific compound 6 for 4 h. Cells were lysed and immunoblotted as described above with antibodies to FGFR2 and β-Actin.

The structure of control-1 is set forth below.

### Control-1

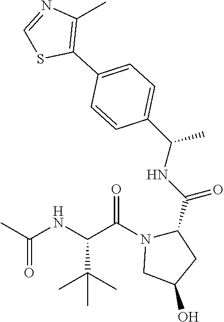 [SMILES: CC(=O)N[C@H](C(=O)N1C[C@H](O)C[C@H]1C(=O)N[C@@H](C)c1ccc(-c2scnc2C)cc1)C(C)(C)C]

The results, illustrated in FIG. 1B, show that BGJ398, control-1, Bortezomib, and MLN4924 rescued the FGFR2 degradation inducing by bispecific compound 6, indicating that the FGFR2 degradation are both ligand and proteasome dependent.

Kato III cells were treated with 0, 0.1, or 0.5 μM bispecific compound 6 for 4, 8, 12, or 16 h. Cells were lysed and immunoblotted as described above with antibodies to FGFR2 and β-Actin. The results, illustrated in FIG. 1C, show that substantial FGFR2 degradation occurred within 4 h and persisted for at least 16 h at the concentrations of 0.1 and 0.5 μM.

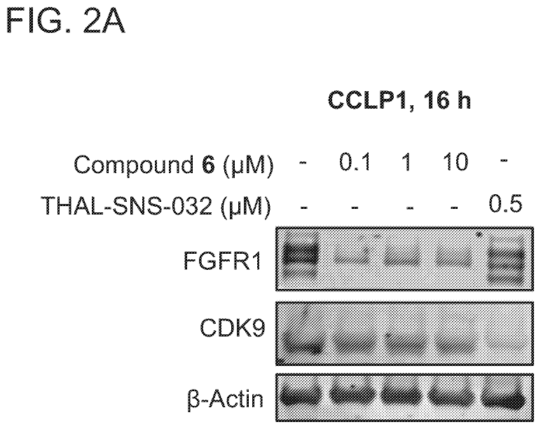

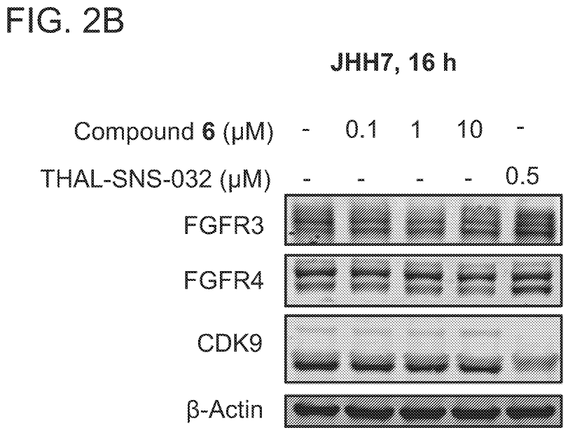

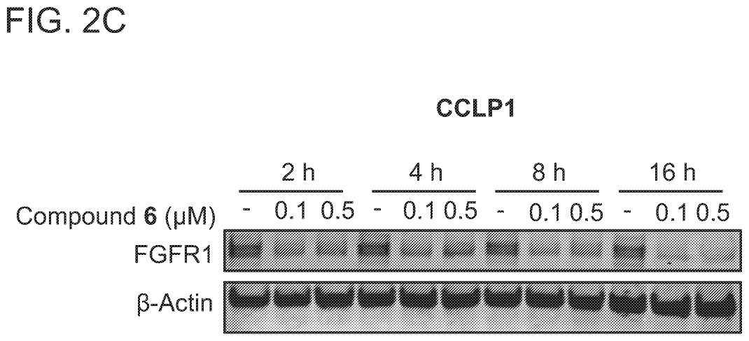

CCLP1 cells were treated with 0, 0.1, 1, or 10 M of bispecific compound 6 or 0.5 μM THAL-SNS-032 for 16 hours, and then lysed and immunoblotted with antibodies to FGFR1 (cell signaling), CDK9, and β-Actin. The results, illustrated in FIG. 2A, indicate that bispecific compound 6 induced the degradation of FGFR1 after 16 hours at the indicated concentrations and THAL-SNS-032 induced CDK9 degradation.

JHH7 cells were treated with 0, 0.1, 1, or 10 M of bispecific compound 6 or 0.5 M THAL-SNS-032 for 16 hours, and then lysed and immunoblotted with antibodies to FGFR3 (cell signaling), FGFR4 (cell signaling), CDK9, and β-Actin. The results, illustrated in FIG. 2B, indicate that bispecific compound 6 did not induce the degradation of FGFR3 and FGFR4 after 16 hours at the indicated concentrations. THAL-SNS-032 induces CDK9 degradation.

CCLP1 cells were treated with 0, 0.1, or 0.5 μM of bispecific compound 6 for 2, 4, 8, or 16 h. Cells were lysed and immunoblotted with antibodies to FGFR1 and β-Actin. The results, illustrated in FIG. 2C, show that substantial FGFR1 degradation occurred within 2 h and persisted for at least 16 h at the concentrations of 0.1 and 0.5 μM.

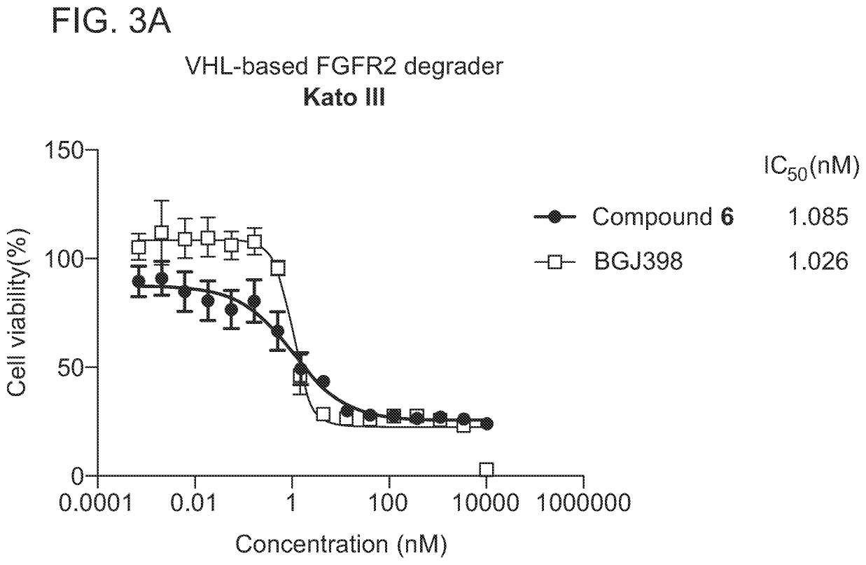

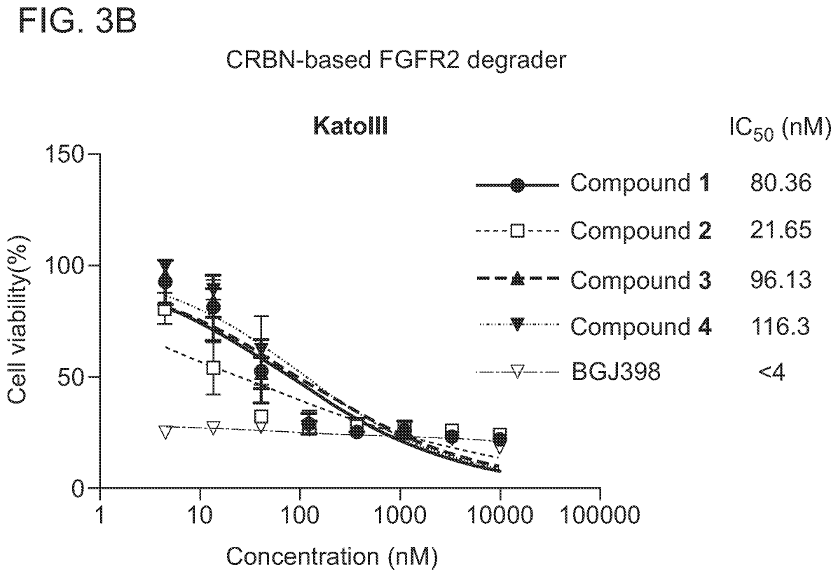

Kato III cells were treated with bispecific compound 6 or BGJ398 at the indicated concentrations for 72 h. Cell viability was determined using Cell-Titer Glo (Promega™) according to the manufacturer's instructions. IC₅₀ values (defined as the concentration of compound needed to reduce cell viability to 50% of the vehicle DMSO control) were estimated by using GraphPad Prism 7.0 (GraphPad Prism®). The results, illustrated in FIG. 3A, show that IC₅₀ values of both bispecific compound 6 and BGJ398 are around 1 nM, indicating that compound 6 has similar potent anti-proliferation effects as BGJ398.

Kato III cells were treated with bispecific compounds 1-4 or BGJ398 at the indicated concentrations for 72 h. Cell viability was determined using Cell-Titer Glo (Promega™) according to the manufacturer's instructions. IC₅₀ values were estimated by using GraphPad Prism 7.0 (GraphPad Prism®). The results, illustrated in FIG. 3B, indicate that bispecific compounds 1-4 and BGJ398 have good anti-proliferation effect.

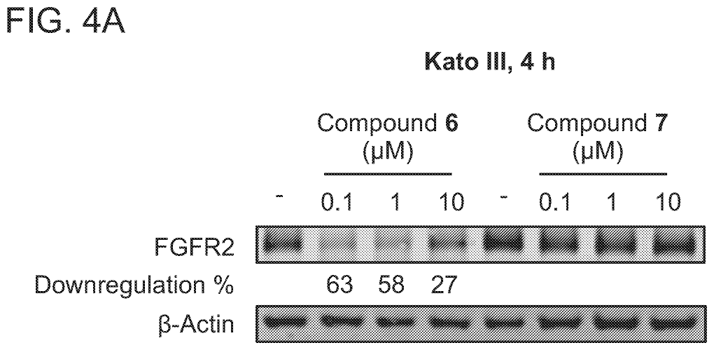

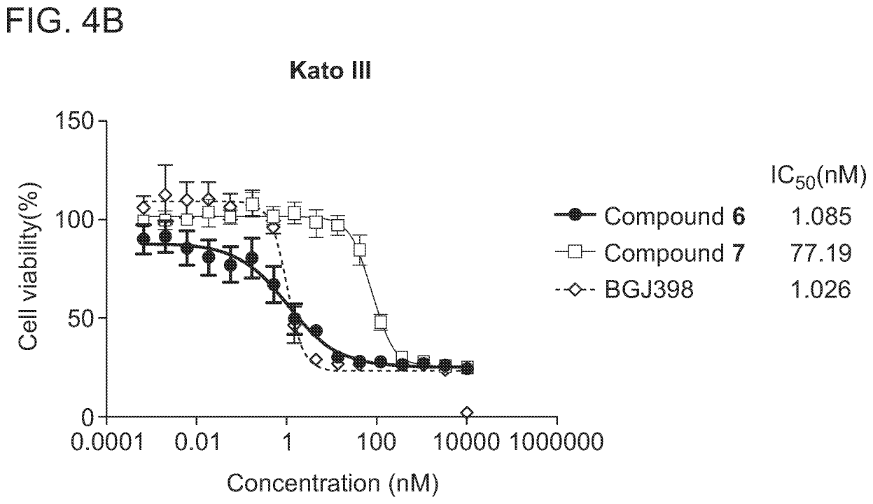

Kato III cells were treated with 0, 0.1, 1 or 10 M of bispecific compound 6 or 7 for 4 h. Cells were lysed and immunoblotted as described above with antibodies to FGFR2 and β-Actin. The results, illustrated in FIG. 4A, indicate that bispecific compound 6 induced the degradation of FGFR2 after 4 hours at the indicated concentrations and bispecific compound 7 did not degrade FGFR2 at the indicated concentrations.

Kato III cells were treated with bispecific compound 6, bispecific compound 7 or BGJ398 at the indicated concentrations for 72 h. Cell viability was determined using Cell-Titer Glo (Promega™), according to the manufacturer's instructions. IC₅₀ values (defined as the concentration of compound needed to reduce cell viability to 50% of the vehicle DMSO control) were estimated by using GraphPad Prism 7.0 (GraphPad Prism®).

The results, illustrated in FIG. 4B, show that IC₅₀ values of both bispecific compound 6 and BGJ398 were around 1 nM, indicating that bispecific compound 6 has similar potent anti-proliferation effects as BGJ398. Bispecific compound 7 (negative control) showed a 77-fold weaker anti-proliferation effect with an IC₅₀ value around 77 nM.

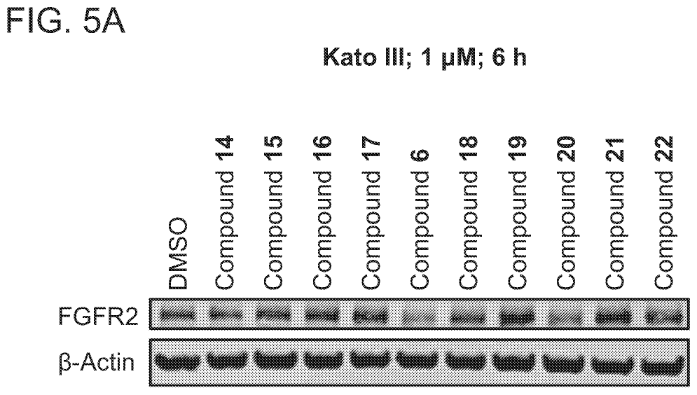

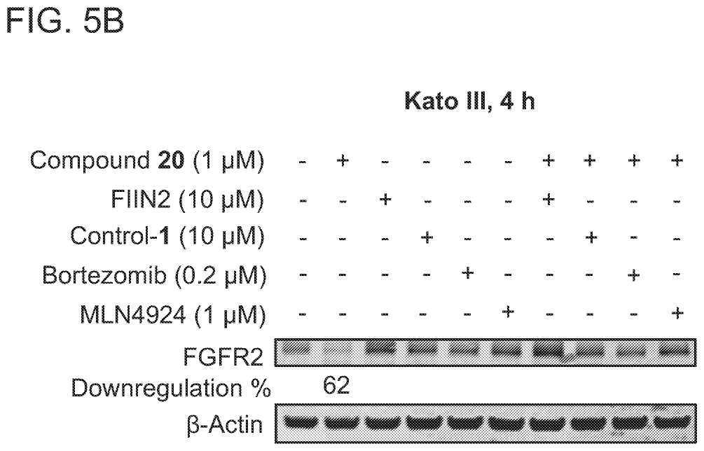

Kato III cells were treated with 0 or 1 μM of bispecific compounds 6 and 14-22 for 6 h. Cells were lysed and immunoblotted as described above with antibodies to FGFR2 and 0-Actin. The results, illustrated in FIG. 5A, indicate that compounds 6 and 20 induced the degradation of FGFR2 after 6 hours at the concentration of 1 M.

Kato III cells were pretreated with 10 μM FIIN2 (the parental compound and known FGFR1/2/3/4 inhibitor; available from, e.g., Selleckchem, Cat. No. S7714, Houston, Tex.), 10 M control-1 (VHL ligand), 0.5 M Bortezomib (a proteasome inhibitor; available from, e.g., Millipore Sigma, Cat. No. 179324-69-7, Burlington, Mass.), and 1 μM MLN4924 (a neddylation inhibitor; available from, e.g., MedChemExpress, Cat. No. HY-70062, Monmouth Junction, N.J.), for 2 h, and then treated with 1 M bispecific compound 20 for 4 h. Cells were lysed and immunoblotted as described above with antibodies to FGFR2 and β-Actin.

The results, illustrated in FIG. 5B, show that FIIN2, control-1, Bortezomib, and MLN4924 rescued the FGFR2 degradation induced by bispecific compound 20. This indicated that FGFR2 degradation is both ligand- and proteasome-dependent.

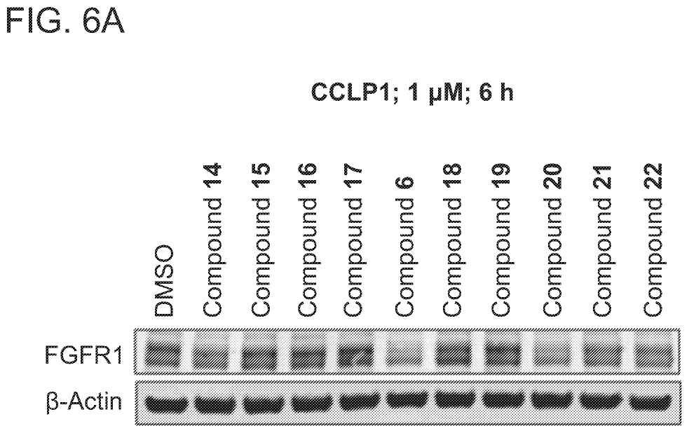

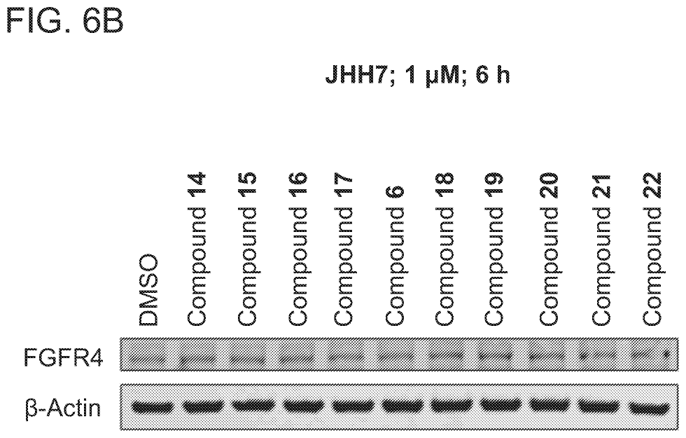

CCLP1 cells were treated with 0 or 1 μM of bispecific compounds 6 and 14-22 for 6 h. Cells were lysed and immunoblotted as described above with antibodies to FGFR1 and β-Actin. The results, illustrated in FIG. 6A, indicate that bispecific compounds 6 and 20 induced the degradation of FGFR1 after 6 hours at the concentration of 1 μM.

JHH7 cells were treated with 0 or 1 M of bispecific compounds 6 and 14-22 for 6 h. Cells were lysed and immunoblotted as described above with antibodies to FGFR4 and $-Actin. The results, illustrated in FIG. 6B, indicate that bispecific compounds 6 and 20 induced the degradation of FGFR4 after 6 hours at the concentration of 1 μM.

All patent publications and non-patent publications are indicative of the level of skill of those skilled in the art to which this invention pertains. All these publications (including any specific portions thereof that are referenced) are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A bispecific compound having a structure represented by formula: 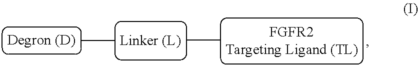
wherein the fibroblast growth factor receptor 2 (FGFR2) targeting ligand is represented by TL-1: 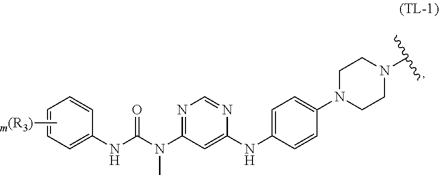
wherein
R₃ is independently halo, optionally substituted alkyl, optionally substituted alkoxy, optionally substituted amino, optionally substituted amido, carboxyl, acrylamide, optionally substituted carbocyclyl, or optionally substituted heterocyclyl; and
m is an integer of 0-4, or
the FGFR2 targeting ligand is represented by TL-2: 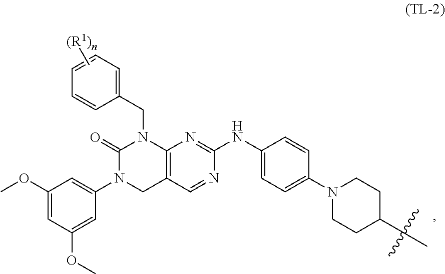
wherein:
R¹ is H or optionally substituted alkyl, optionally substituted alkoxy, optionally substituted amino, optionally substituted amido, carboxyl, acrylamide, optionally substituted carbocyclyl, or optionally substituted heterocyclyl; and
n is an integer 0-4, or
the FGFR2 targeting ligand is represented by TL-3a or TL-3b: 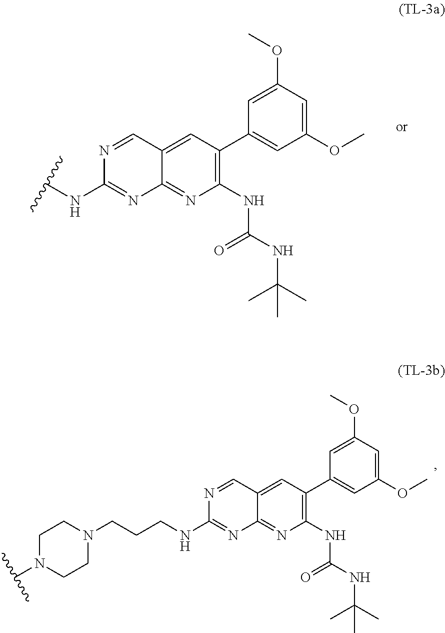 [SMILES: *Nc1ncc2cc(-c3cc(OC)cc(OC)c3)c(NC(=O)NC(C)(C)C)nc2n1 ; *N1CCN(CCCNc2ncc3cc(-c4cc(OC)cc(OC)c4)c(NC(=O)NC(C)(C)C)nc3n2)CC1]
or
the FGFR2 targeting ligand is represented by TL-4: 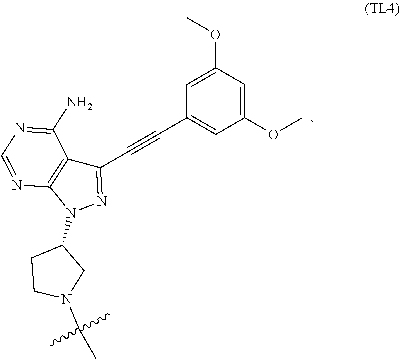 [SMILES: *N1CC[C@H](n2nc(C#Cc3cc(OC)cc(OC)c3)c3c(N)ncnc32)C1]
the degron represents a moiety that binds an E3 ubiquitin ligase, and the linker represents a moiety that covalently connects the degron and the targeting ligand, or a pharmaceutically acceptable salt or stereoisomer thereof.

2. The bispecific compound of claim 1, which is represented by the formula (I-1): 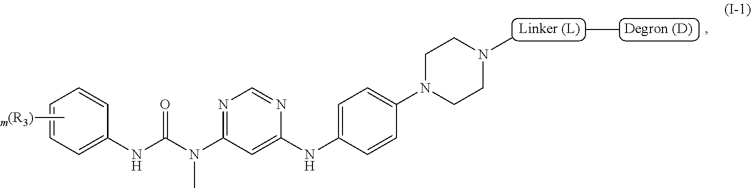
wherein
R₃ is independently halo, optionally substituted alkyl, optionally substituted alkoxy, optionally substituted amino, optionally substituted amido, carboxyl, acrylamide, optionally substituted carbocyclyl, or optionally substituted heterocyclyl; and
m is an integer 0-4.

3. The bispecific compound of claim 2, wherein R₃ is independently methyl, chloro, or methoxy.

4. The bispecific compound of claim 2, wherein m is 0.

5. The bispecific compound of claim 2, wherein m is 2.

6. The bispecific compound of claim 2, wherein m is 4.

7. The bispecific compound of claim 2, which is represented by formula I-1a, I-1b, I-1c, I-1d, or I-1e: 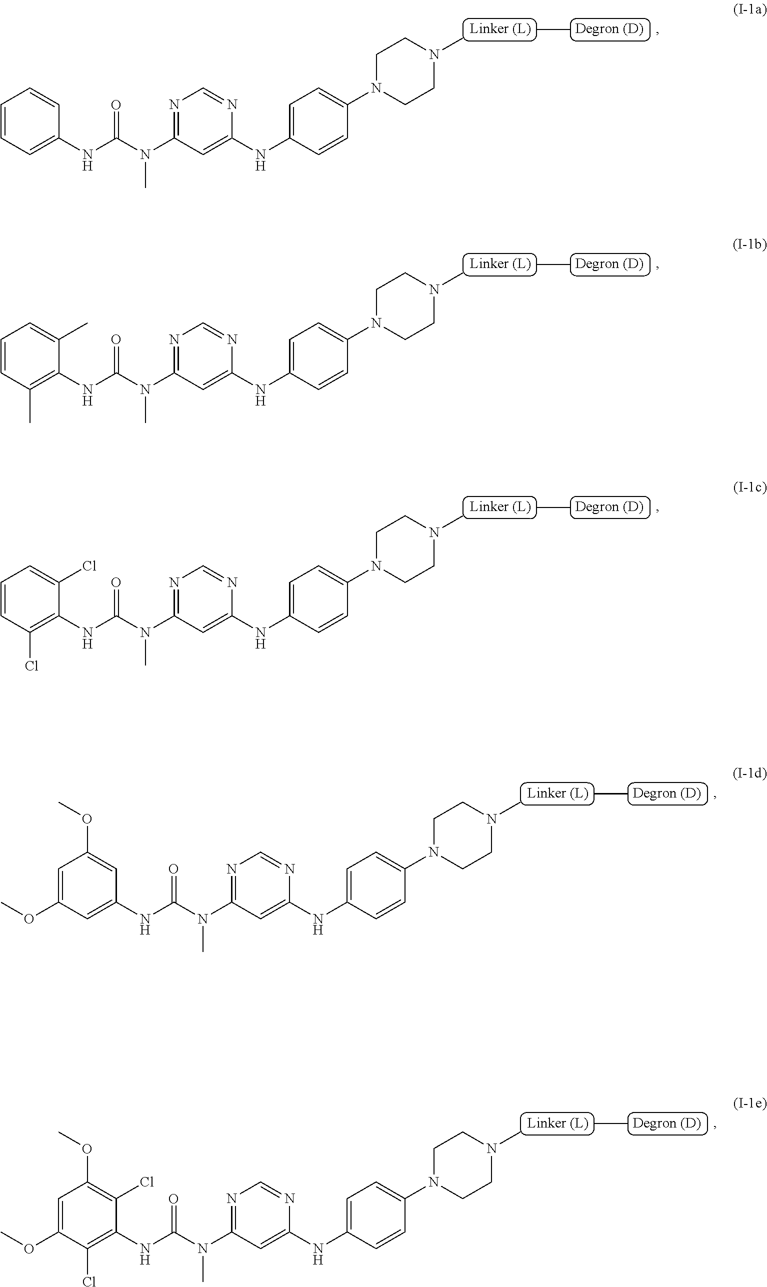
or a pharmaceutically acceptable salt or stereoisomer thereof.

8. The bispecific compound of claim 1, which is represented by the formula (I-2): 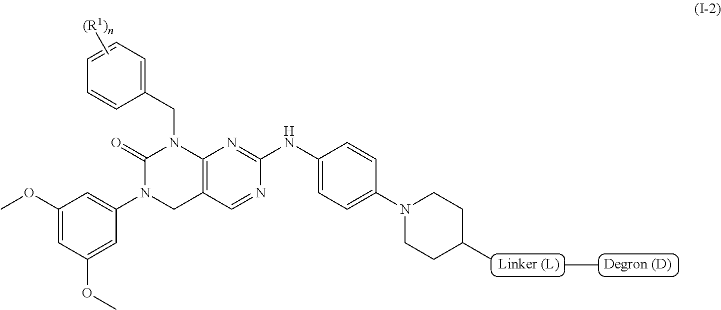
wherein:
R¹ is H or optionally substituted alkyl, optionally substituted alkoxy, optionally substituted amino, optionally substituted amido, carboxyl, acrylamide, optionally substituted carbocyclyl, or optionally substituted heterocyclyl; and
n is an integer 0-4;
or a pharmaceutically acceptable salt or stereoisomer thereof.

9. The bispecific compound of claim 8, which is represented by the formula (I-2a): 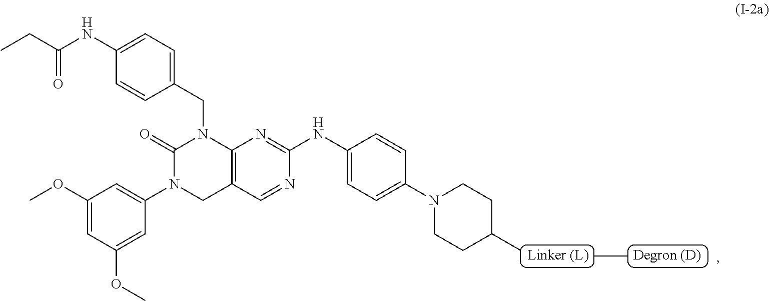
or a pharmaceutically acceptable salt or stereoisomer thereof.

10. The bispecific compound of claim 1, which is represented by the formula (I-3a) or (I-3b): 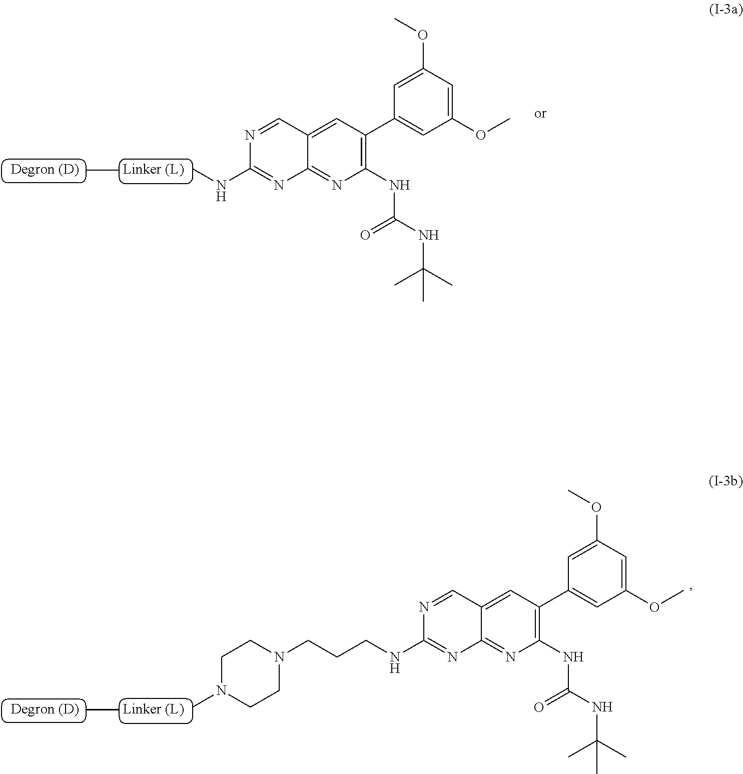
or a pharmaceutically acceptable salt or stereoisomer thereof.

11. The bispecific compound of claim 1, which is represented by the formula (I-4): 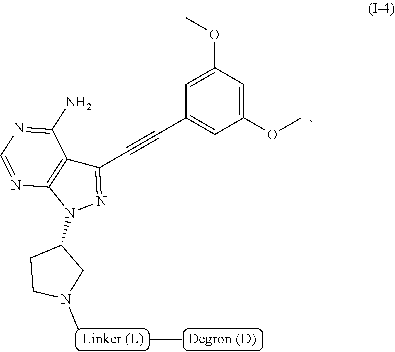
or a pharmaceutically acceptable salt or stereoisomer thereof.

12. The bispecific compound of claim 1, wherein the linker is an alkylene chain or a bivalent alkylene chain, either of which may be interrupted by, and/or terminate at either or both termini in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃₋₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

13. The bispecific compound of claim 12, wherein the linker is an alkylene chain having 1-10 alkylene units and interrupted by or terminating in

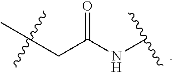 [SMILES: *CC(=O)N*]

14. The bispecific compound of claim 1, wherein the linker is a polyethylene glycol chain which may terminate at either or both termini in at least one of —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃₋₁₂ carbocyclene, 3- to 12-membered heterocyclene, 5- to 12-membered heteroarylene or any combination thereof, wherein R′ is H or C₁-C₆ alkyl, wherein the one or both terminating groups may be the same or different.

15. The bispecific compound of claim 14, wherein the linker is a polyethylene glycol linker having 2-8 PEG units and terminating in

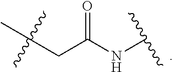 [SMILES: *CC(=O)N*]

16. The bispecific compound of claim 1, which is represented by any one of the following structures: 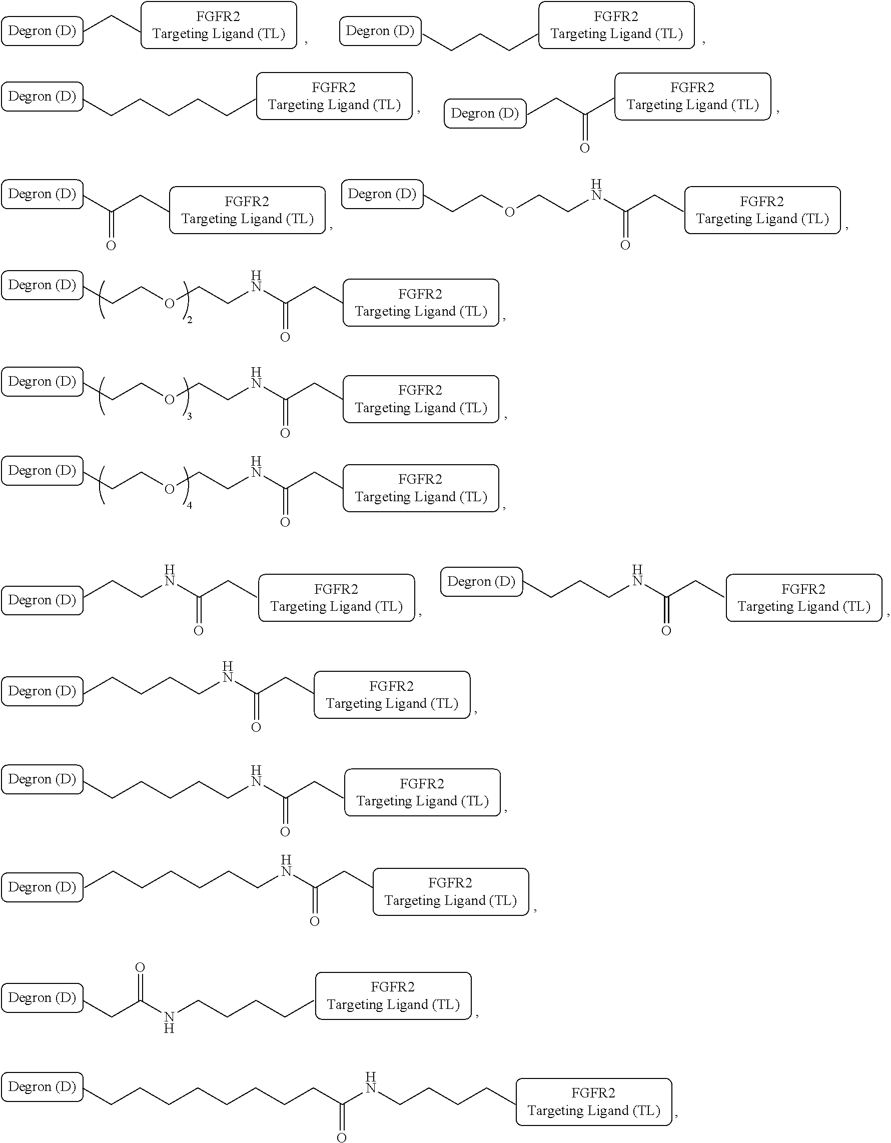
or a pharmaceutically acceptable salt or stereoisomer thereof.

17. The bispecific compound of claim 1, wherein the degron binds cereblon, wherein the degron is represented by the formula D1-a or D1-b: 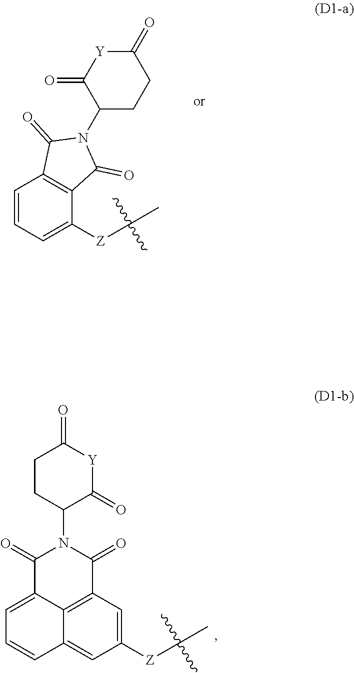
wherein
Y is NH or O; and
Z is NH, O, or C≡.

18. (canceled)

19. (canceled)

20. The bispecific compound of claim 1, wherein the degron binds von Hippel-Landau (VHL), wherein the degron is represented by any one of the structures: 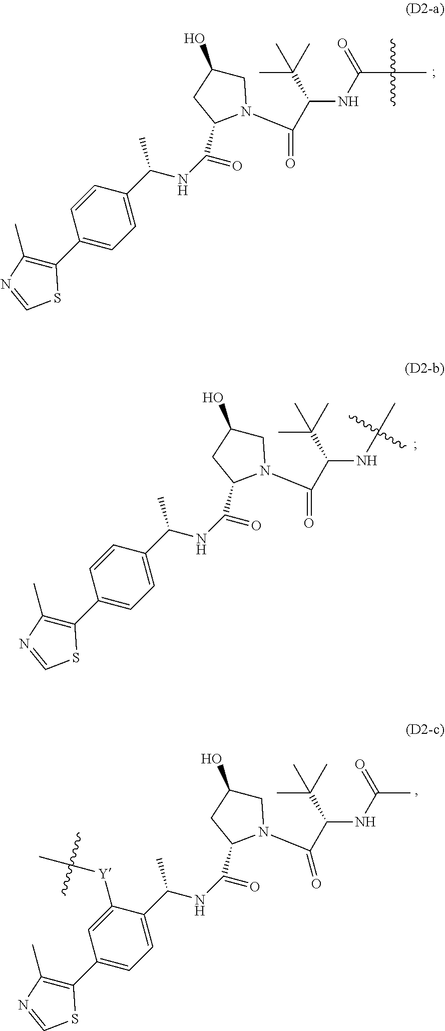
wherein Y′ is a bond, N, O or C; 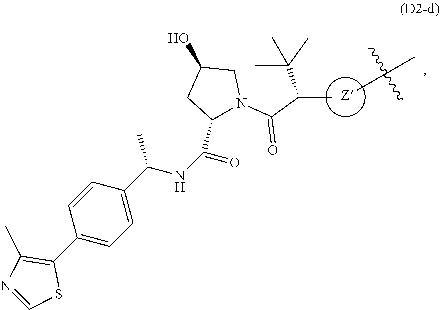
wherein Z′ is a cyclic group; 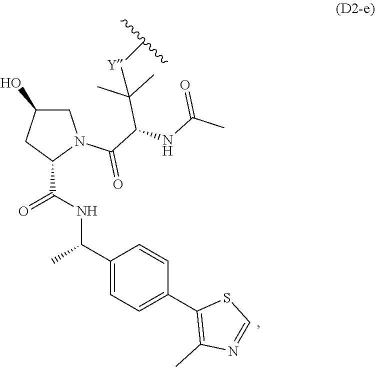
wherein Y″ is a bond, CH₂, NH, NMe, O, or S; or stereoisomer thereof.

21. (canceled)

22. (canceled)

23. The bispecific compound of claim 1, wherein the degron binds an inhibitor of apoptosis protein (IAP), wherein the degron is represented by any one of the structures: 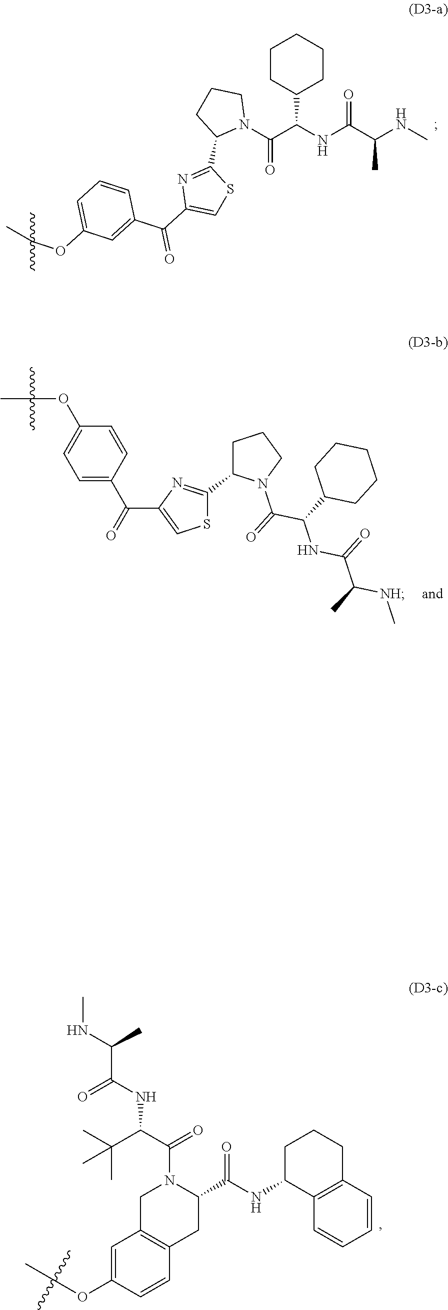 [SMILES: *Oc1cccc(C(=O)c2csc([C@@H]3CCCN3C(=O)[C@@H](NC(=O)[C@H](C)NC)C3CCCCC3)n2)c1 ; *Oc1ccc(C(=O)c2csc([C@@H]3CCCN3C(=O)[C@@H](NC(=O)[C@H](C)NC)C3CCCCC3)n2)cc1 ; "and" ; *Oc1ccc2c(c1)CN(C(=O)[C@@H](NC(=O)[C@H](C)NC)C(C)(C)C)[C@H](C(=O)N[C@@H]1CCCc3ccccc31)C2]
or stereoisomer thereof.

24. (canceled)

25. (canceled)

26. The bispecific compound of claim 1, which is: 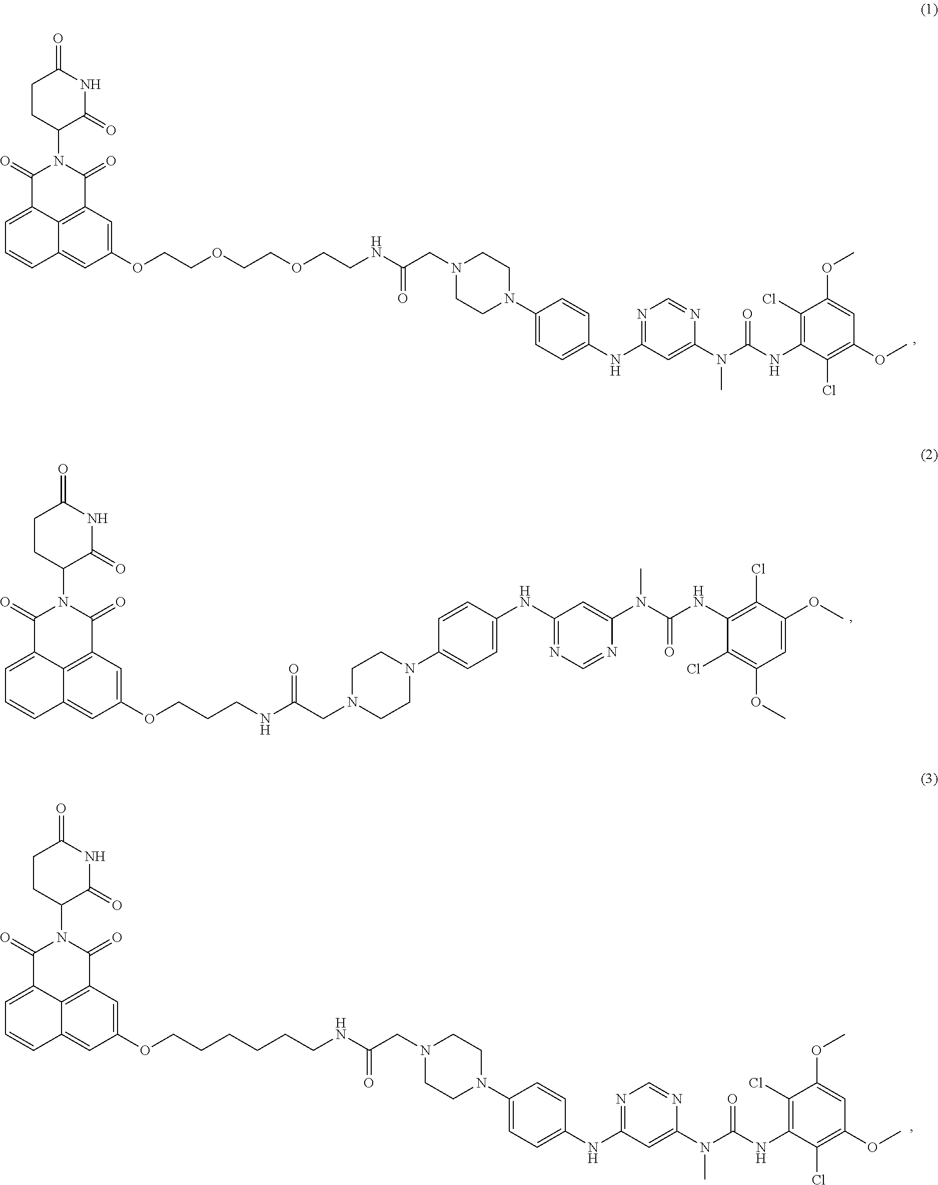 [SMILES: COc1cc(OC)c(Cl)c(NC(=O)N(C)c2cc(Nc3ccc(N4CCN(CC(=O)NCCOCCOCCOc5cc6c7c(cccc7c5)C(=O)N(C5CCC(=O)NC5=O)C6=O)CC4)cc3)ncn2)c1Cl ; COc1cc(OC)c(Cl)c(NC(=O)N(C)c2cc(Nc3ccc(N4CCN(CC(=O)NCCCOc5cc6c7c(cccc7c5)C(=O)N(C5CCC(=O)NC5=O)C6=O)CC4)cc3)ncn2)c1Cl ; COc1cc(OC)c(Cl)c(NC(=O)N(C)c2cc(Nc3ccc(N4CCN(CC(=O)NCCCCCCOc5cc6c7c(cccc7c5)C(=O)N(C5CCC(=O)NC5=O)C6=O)CC4)cc3)ncn2)c1Cl] 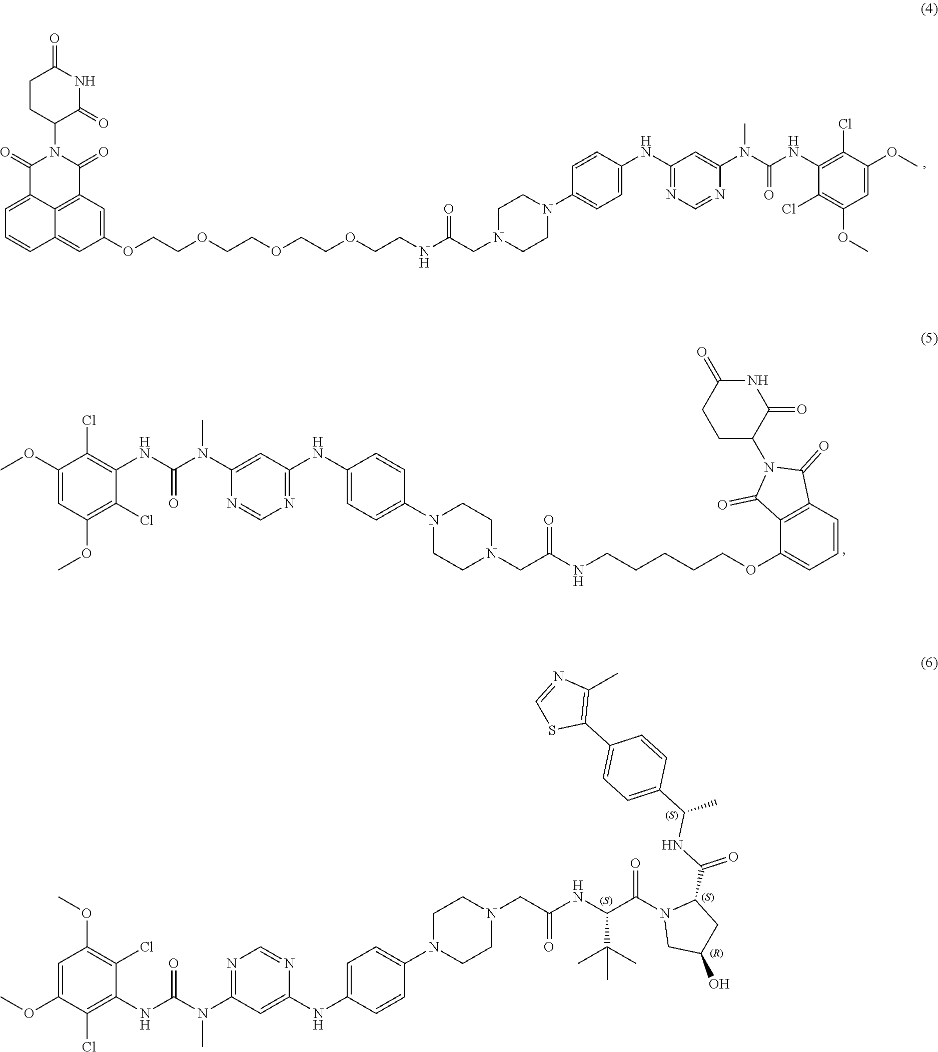 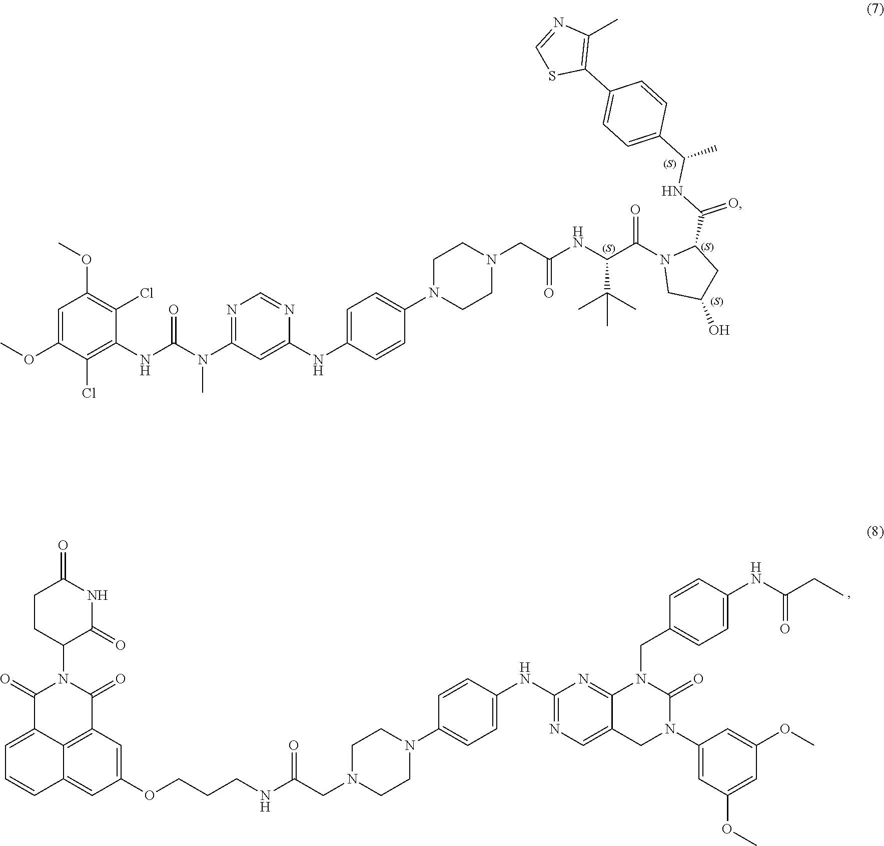 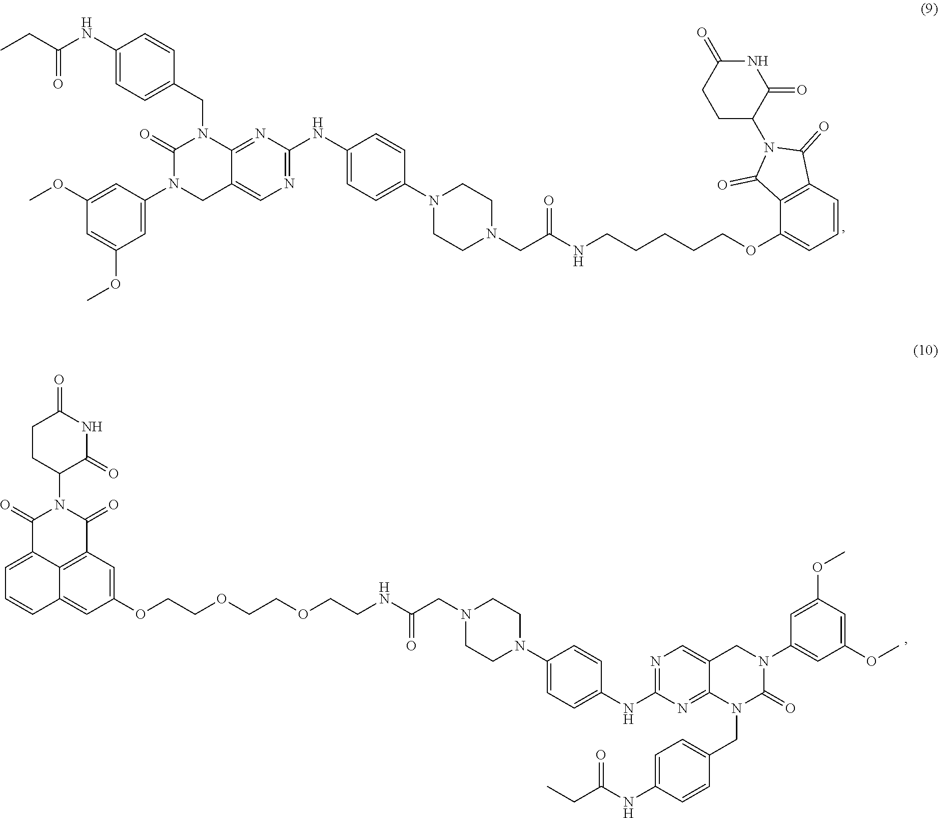 [SMILES: CCC(=O)Nc1ccc(CN2C(=O)N(c3cc(OC)cc(OC)c3)Cc3cnc(Nc4ccc(N5CCN(CC(=O)NCCCCCOc6cccc7c6C(=O)N(C6CCC(=O)NC6=O)C7=O)CC5)cc4)nc32)cc1 ; CCC(=O)Nc1ccc(CN2C(=O)N(c3cc(OC)cc(OC)c3)Cc3cnc(Nc4ccc(N5CCN(CC(=O)NCCOCCOCCOc6cc7c8c(cccc8c6)C(=O)N(C6CCC(=O)NC6=O)C7=O)CC5)cc4)nc32)cc1] 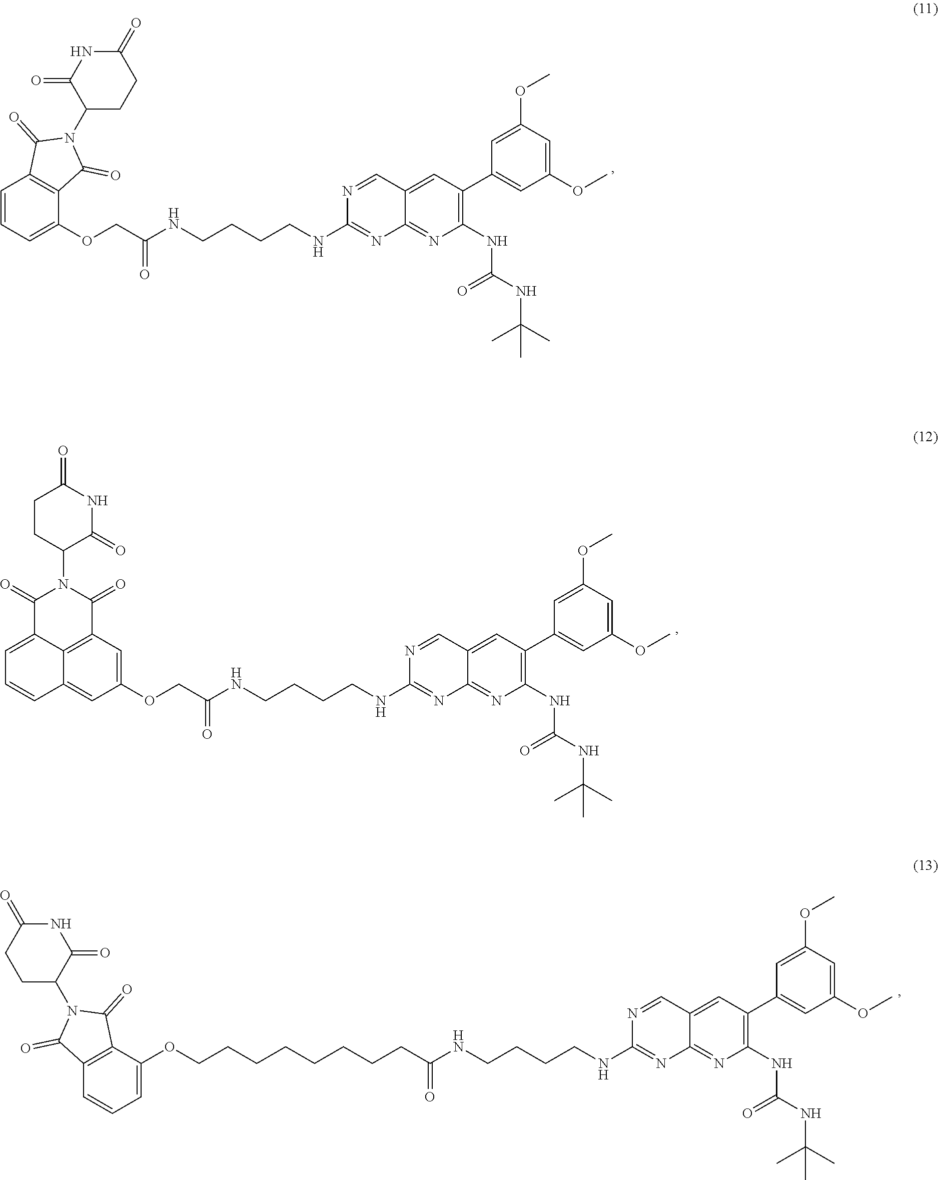 [SMILES: COc1cc(OC)cc(-c2cc3cnc(NCCCCNC(=O)COc4cccc5c4C(=O)N(C4CCC(=O)NC4=O)C5=O)nc3nc2NC(=O)NC(C)(C)C)c1 ; COc1cc(OC)cc(-c2cc3cnc(NCCCCNC(=O)COc4cc5c6c(cccc6c4)C(=O)N(C4CCC(=O)NC4=O)C5=O)nc3nc2NC(=O)NC(C)(C)C)c1 ; COc1cc(OC)cc(-c2cc3cnc(NCCCCNC(=O)CCCCCCCCOc4cccc5c4C(=O)N(C4CCC(=O)NC4=O)C5=O)nc3nc2NC(=O)NC(C)(C)C)c1] 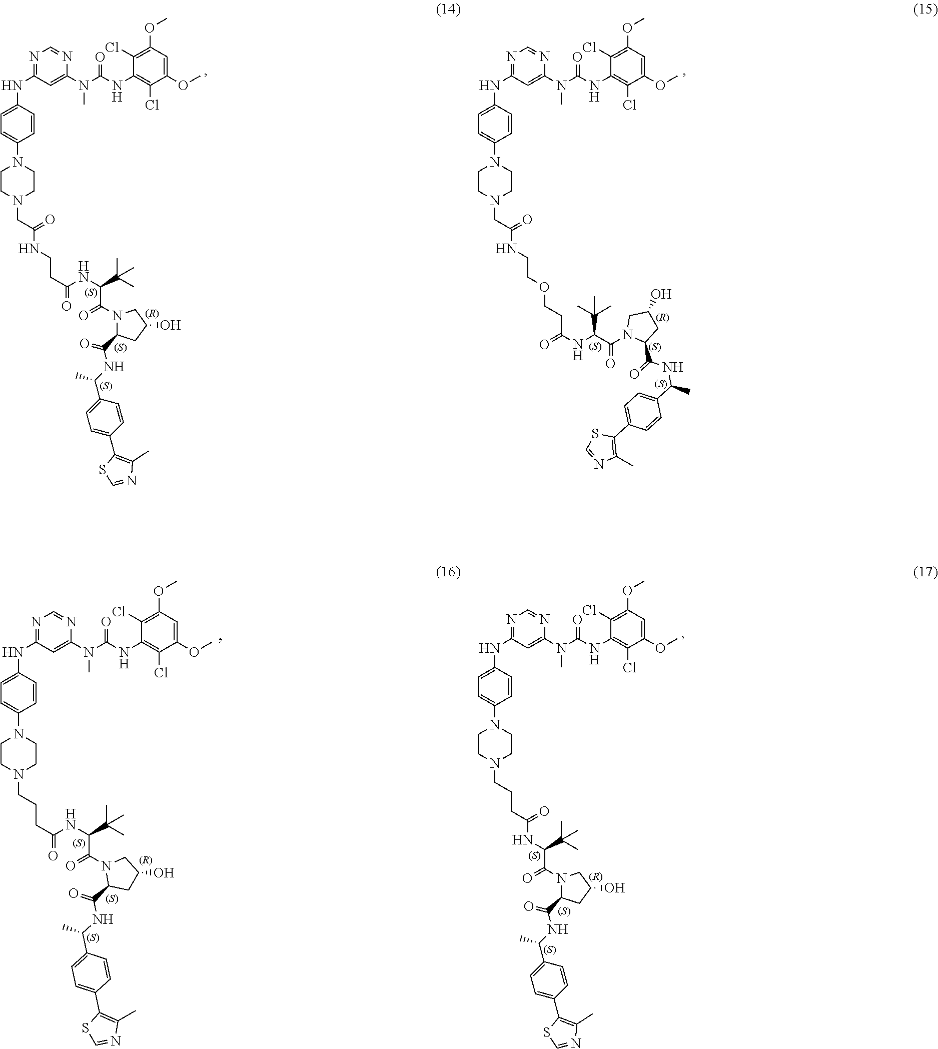 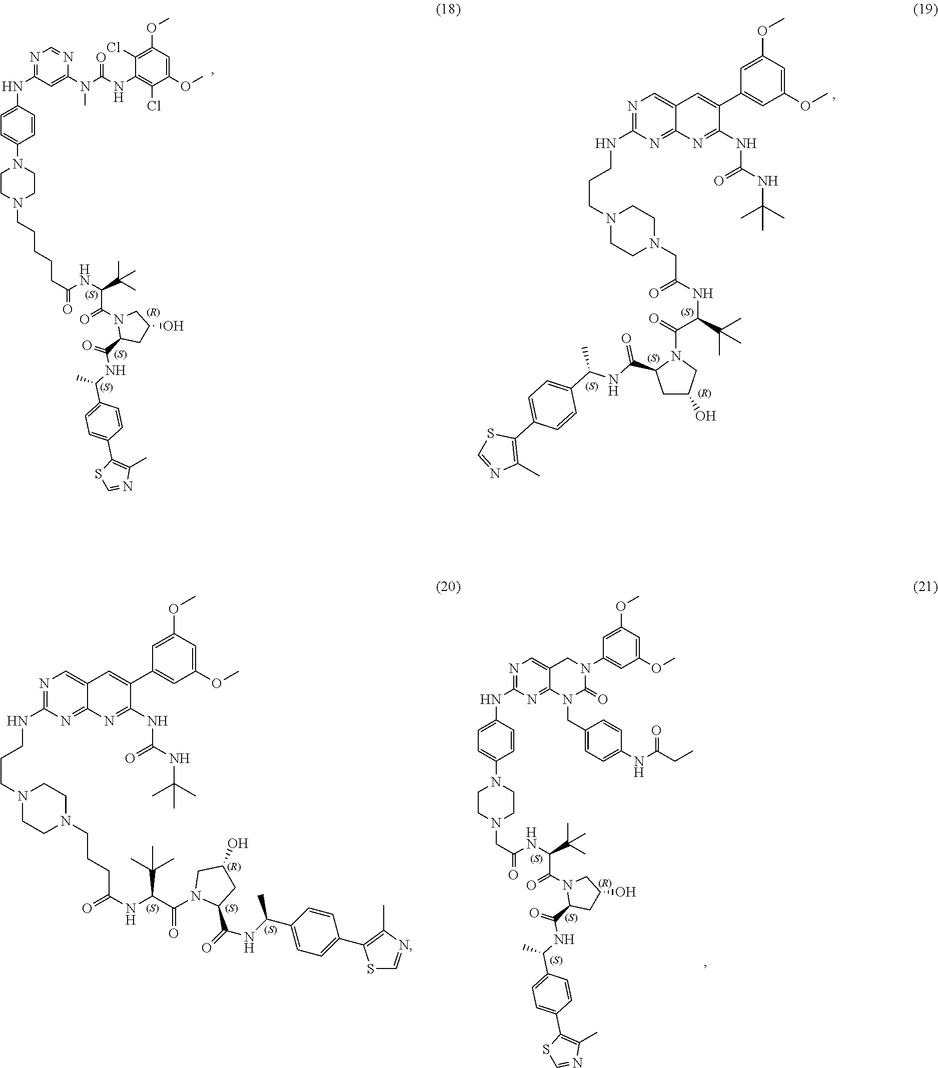 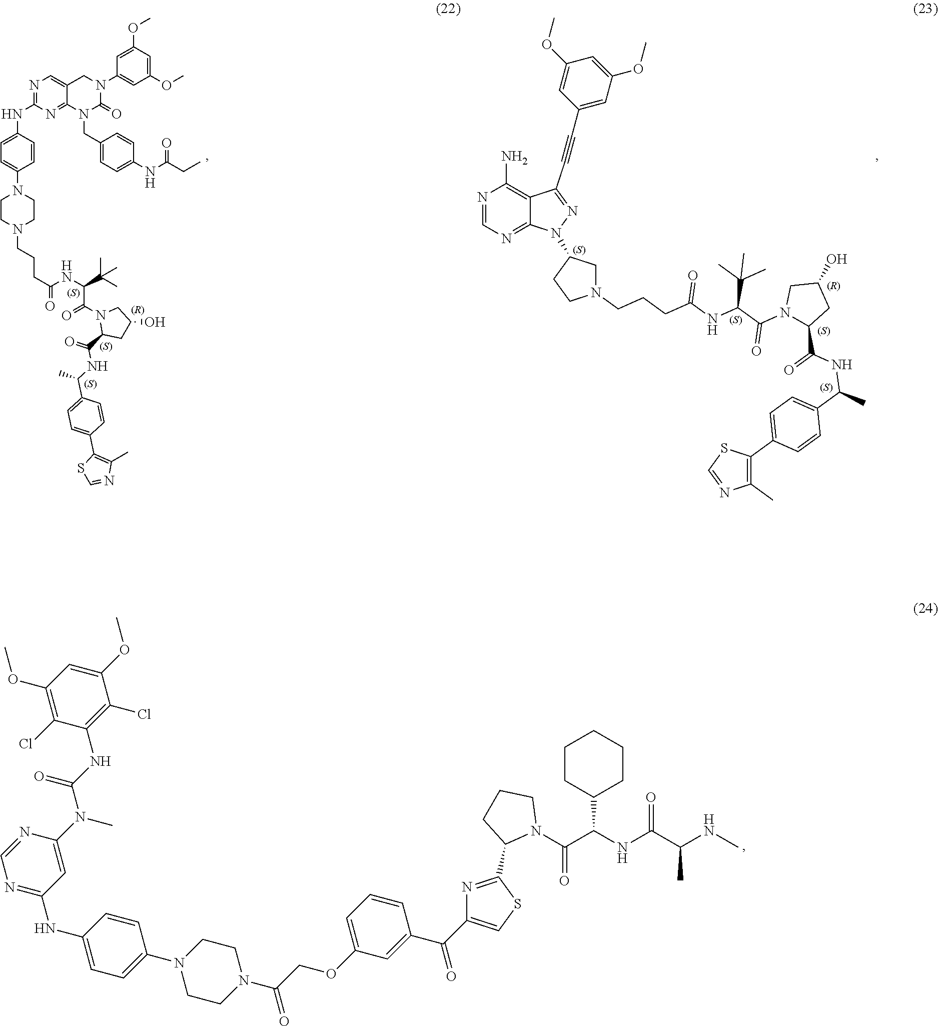 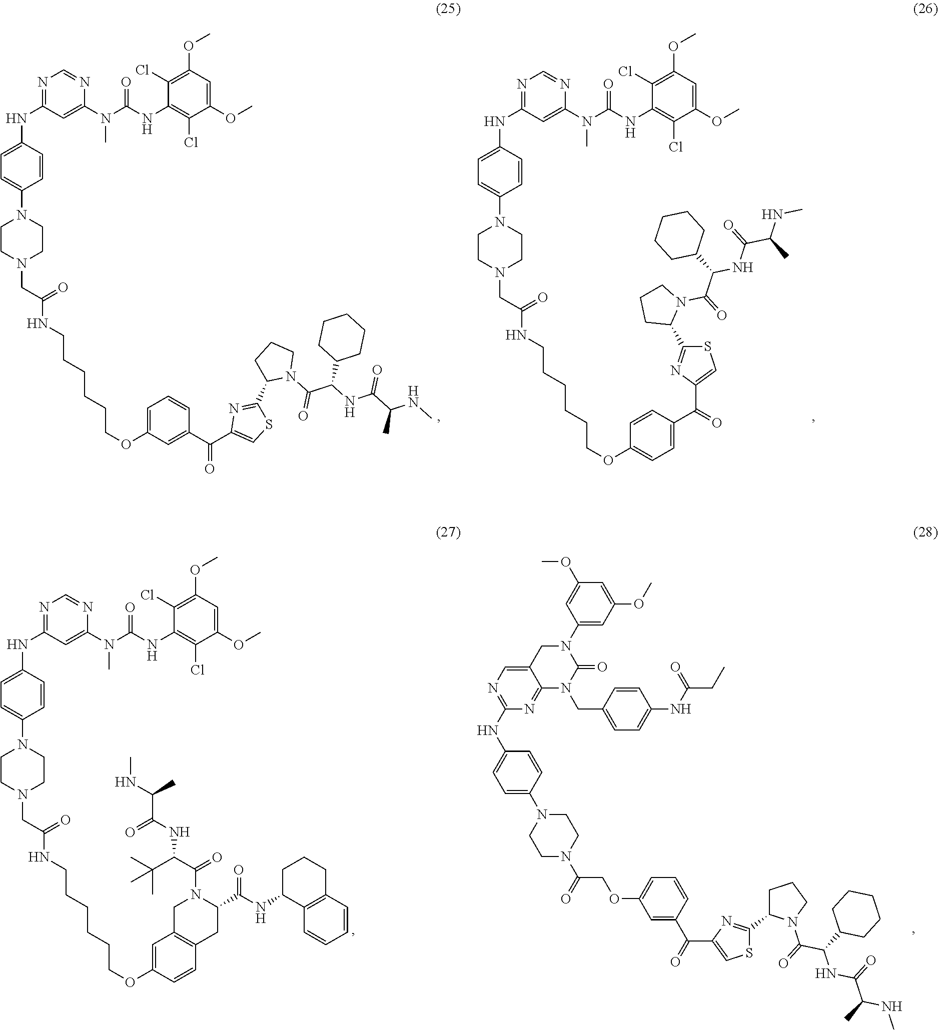 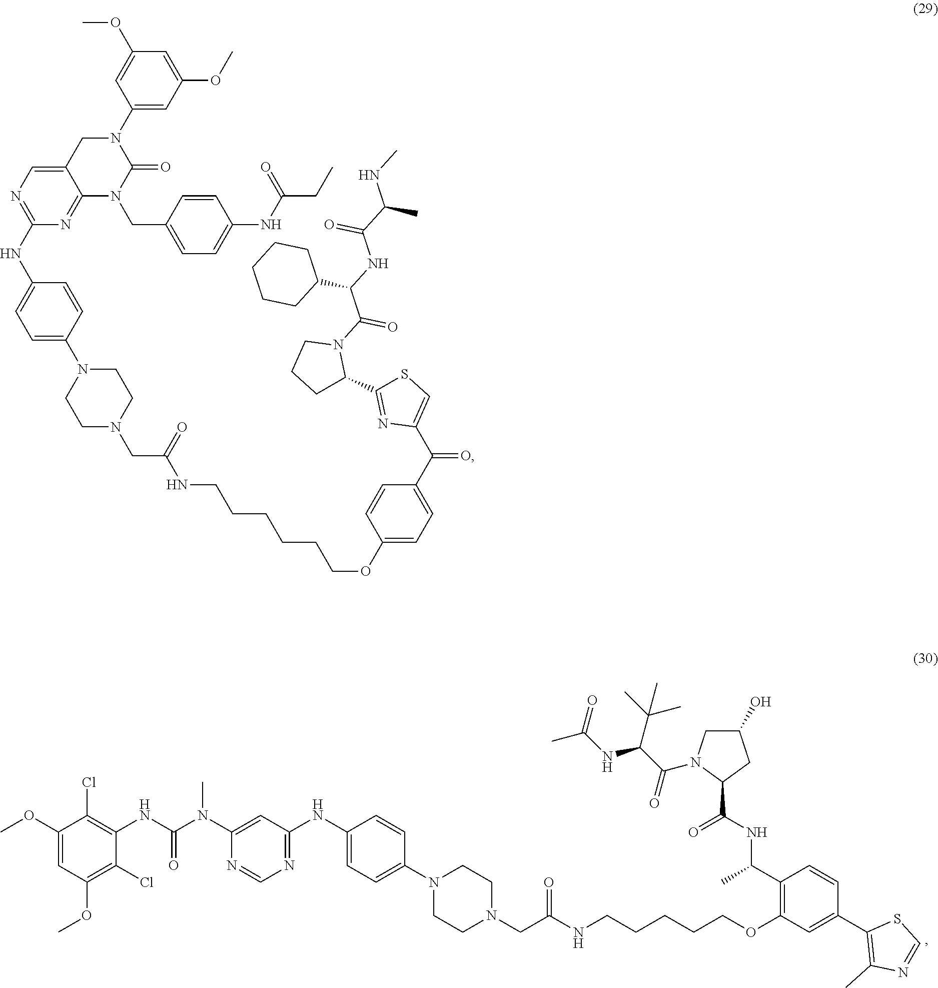 [SMILES: CCC(=O)Nc1ccc(CN2C(=O)N(c3cc(OC)cc(OC)c3)Cc3cnc(Nc4ccc(N5CCN(CC(=O)NCCCCCCOc6ccc(C(=O)c7csc([C@@H]8CCCN8C(=O)[C@@H](NC(=O)[C@H](C)NC)C8CCCCC8)n7)cc6)CC5)cc4)nc32)cc1 ; COc1cc(OC)c(Cl)c(NC(=O)N(C)c2cc(Nc3ccc(N4CCN(CC(=O)NCCCCCOc5cc(-c6scnc6C)ccc5[C@H](C)NC(=O)[C@@H]5C[C@@H](O)CN5C(=O)[C@@H](NC(C)=O)C(C)(C)C)CC4)cc3)ncn2)c1Cl] 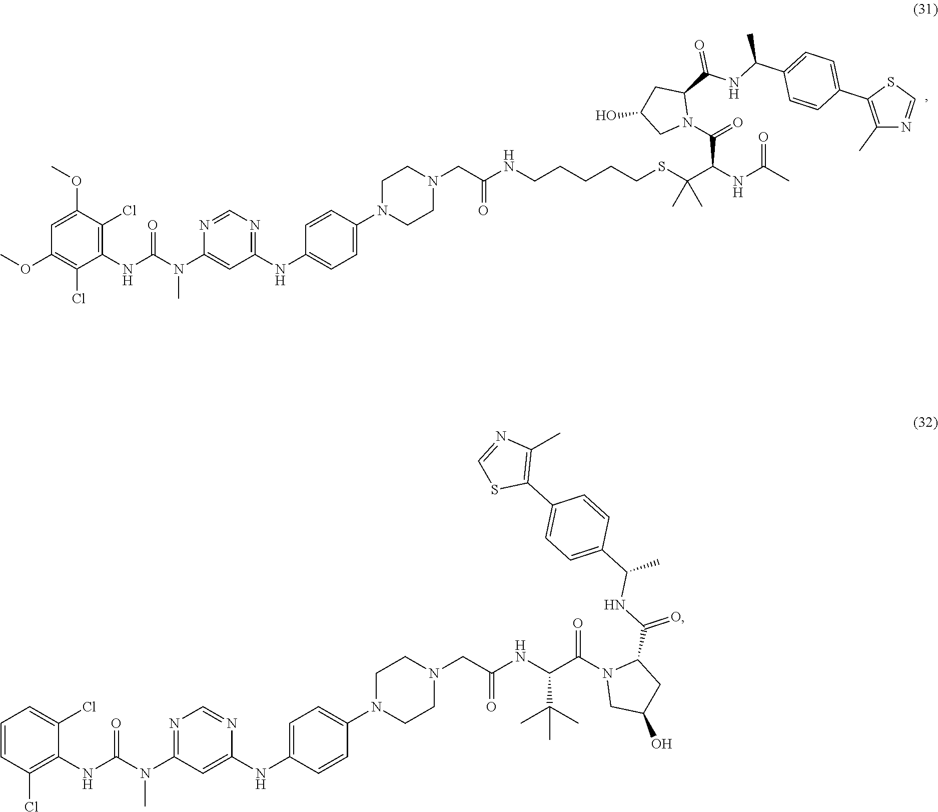 [SMILES: COc1cc(OC)c(Cl)c(NC(=O)N(C)c2cc(Nc3ccc(N4CCN(CC(=O)NCCCCCSC(C)(C)[C@H](NC(C)=O)C(=O)N5C[C@H](O)C[C@H]5C(=O)N[C@@H](C)c5ccc(-c6scnc6C)cc5)CC4)cc3)ncn2)c1Cl ; Cc1ncsc1-c1ccc([C@H](C)NC(=O)[C@@H]2C[C@@H](O)CN2C(=O)[C@@H](NC(=O)CN2CCN(c3ccc(Nc4cc(N(C)C(=O)Nc5c(Cl)cccc5Cl)ncn4)cc3)CC2)C(C)(C)C)cc1] 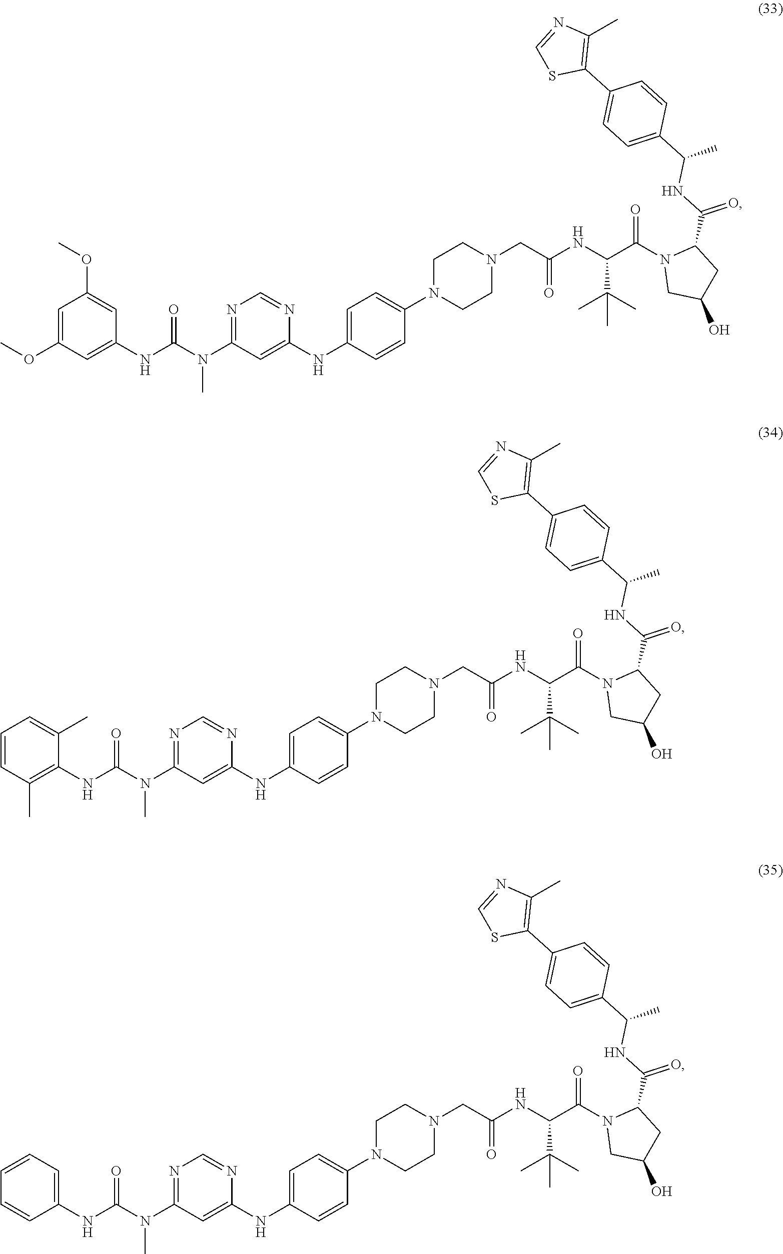 [SMILES: COc1cc(NC(=O)N(C)c2cc(Nc3ccc(N4CCN(CC(=O)N[C@H](C(=O)N5C[C@H](O)C[C@H]5C(=O)N[C@@H](C)c5ccc(-c6scnc6C)cc5)C(C)(C)C)CC4)cc3)ncn2)cc(OC)c1 ; Cc1cccc(C)c1NC(=O)N(C)c1cc(Nc2ccc(N3CCN(CC(=O)N[C@H](C(=O)N4C[C@H](O)C[C@H]4C(=O)N[C@@H](C)c4ccc(-c5scnc5C)cc4)C(C)(C)C)CC3)cc2)ncn1 ; Cc1ncsc1-c1ccc([C@H](C)NC(=O)[C@@H]2C[C@@H](O)CN2C(=O)[C@@H](NC(=O)CN2CCN(c3ccc(Nc4cc(N(C)C(=O)Nc5ccccc5)ncn4)cc3)CC2)C(C)(C)C)cc1]
or a pharmaceutically acceptable salt or stereoisomer thereof.

27. A pharmaceutical composition, comprising a therapeutically effective amount of the bispecific compound of claim 1, or pharmaceutically acceptable salt or stereoisomer thereof, and a pharmaceutically acceptable carrier.

28. The method of treating a disease or disorder that is characterized or mediated by aberrant activity of FGFR2, comprising administering to a subject in need thereof a therapeutically effective amount of the bispecific compound of claim 1, or a pharmaceutically acceptable salt or stereoisomer thereof.

29. The method of claim 28, wherein the disease or disorder is cancer.

30. The method of claim 29, wherein the cancer is liver cancer.

31. The method of claim 31, wherein the cancer is biliary tract cancer (BTC).

32. The method of claim 32, wherein the BTC is intrahepatic cholangiocarcinoma (ICC) or extrahepatic cholangiocarcinoma (ECC).
